SEQUENCE LISTING (8 sequences)

SEQ ID NO: 1 - DNA, 19 bp, Artificial
  Synthetic - made in laboratory
ggcccagctccagacttga

SEQ ID NO: 2 - DNA, 20 bp, Artificial
  Synthetic - made in laboratory
ctcaggtccccgaactcatg

SEQ ID NO: 3 - DNA, 19 bp, Artificial
  Synthetic - made in laboratory
ttcctggcgttaccttggt

SEQ ID NO: 4 - DNA, 17 bp, Artificial
  Synthetic - made in laboratory
gccactgccggacaact

SEQ ID NO: 5 - DNA, 24 bp, Artificial
  Synthetic - made in laboratory
gaaaacctattccatcgactgtga

SEQ ID NO: 6 - DNA, 22 bp, Artificial
  Synthetic - made in laboratory
acctgacagcggcttatgattt

SEQ ID NO: 7 - DNA, 17 bp, Artificial
  Synthetic - made in laboratory
agtcagccggccagcat

SEQ ID NO: 8 - DNA, 25 bp, Artificial
  Synthetic - made in laboratory
gaagctatctgcaacagtccttcac

(19) United States
(12) Patent Application Publication
(10) Pub. No.: US 2015/0196500 A1
(43) Pub. Date: Jul. 16, 2015

(54) Treatment of Mitochondria-Related Diseases and Improvement of Age-Related Metabolic Deficits

(71) Applicants: Garth James, Smith COOPER, Auckland (NZ); Anthony Ronald, John PHILLIPS, Auckland (NZ); Nancy Xiuyin CHEN, Auckland (NZ); Deming GONG, Auckland (NZ); Maria JULLIG, Auckland (NZ); Anthony John, Rodney HICKEY, Auckland (NZ); Sarah GLYN-JONES, Auckland (NZ)

(72) Inventors: Garth James, Smith COOPER, Auckland (NZ); Anthony Ronald, John PHILLIPS, Auckland (NZ); Nancy Xiuyin CHEN, Auckland (NZ); Deming GONG, Auckland (NZ); Maria JULLIG, Auckland (NZ); Anthony John, Rodney HICKEY, Auckland (NZ); Sarah GLYN-JONES, Auckland (NZ)

(73) Assignee: PHILERA NEW ZEALAND LIMITED, Auckland (NZ)

(21) Appl. No.: 14/571,268

(22) Filed: Dec. 15, 2014

Related U.S. Application Data
(63) Continuation of application No. 13/660,534, filed on Oct. 25, 2012, now abandoned.
Application No. 13/660,534 is a continuation of application No. 12/093,302, filed on Feb. 26, 2010, now abandoned.
(60) Provisional application No. 60/735,688, filed on Nov. 9, 2005.
(60) Provisional application No. 60/739,728, filed on Nov. 23, 2005.

(51) Int. Cl. A61K 31/132

(52) U.S. Cl. CPC A61K 31/132

(57) ABSTRACT

Pharmaceutical compositions and methods for the treatment of subjects, including humans, who have or are at risk for various disease, disorders and conditions, including, mitochondria-associated diseases, disorders, and conditions, including respiratory chain disorders, and diseases, disorders and conditions associated with or characterized at least in part by mitochondria swelling, mitochondria dysfunction, mitochondria leaking, oxidative stress, increased mitochondria number, increased mitochondria and mitochondria-related protein mass, and increased mitochondria and related-related proteins expression.

### CROSS-REFERENCE TO RELATED APPLICATION

This application is a continuation of U.S. application Ser. No. 13/660,534, filed Oct. 25, 2012, which is a continuation of U.S. application Ser. No. 12/093,302, filed Feb. 26, 2010, which claims the benefit of International Application No. PCT/NZ2006/000288, filed Nov. 9, 2006 and published in English on May 18, 2007 as WO 2007/055598 A1, which claims the benefit of U.S. Provisional Application 60/735,688, filed Nov. 9, 2005, and U.S. Provisional Application 60/739,728, filed Nov. 23, 2005; all of which are hereby incorporated by reference in their entirety to the extent not inconsistent with the disclosure herein.

### FIELD OF THE INVENTION

The present inventions relate generally to compounds, compositions and methods of treatment. The present inventions include compounds, compositions and methods for treating mitochondria-associated diseases, including respiratory chain disorders, for improving age-related physiological deficits and increasing longevity, and delaying mitochondrial dysfunction occurring in a mammal during aging.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art.

One of the changes that occur with various disease states, as well as aging, is a change in mitochondria and mitochondrial function. Mitochondria are the cellular organelles that generate energy from aerobic (oxygen-utilizing) metabolism, and are the main energy source in cells of higher organisms.

Most animal cells contain between a few hundred and a few thousand mitochondria, and they are the only cellular organelles with their own DNA. There is no other cellular DNA outside the nucleus apart from the DNA of mitochondria. The human mitochondrion generally contains 5 to 10 circular molecules of DNA. Each consists of 16,569 base pairs carrying the information for 37 genes, which encode 2 different molecules of ribosomal RNA (rRNA), 22 different molecules of transfer RNA (tRNA) (at least one for each amino acid), and 13 polypeptides. The rRNA and tRNA molecules are used in the machinery that synthesizes the 13 polypeptides. The 13 polypeptides are subunits of the protein complexes in the inner mitochondrial membrane, described below. However, each of these protein complexes also requires subunits that are encoded by nuclear genes, which are synthesized on free ribosomes in the cytosol, and imported from the cytosol into the mitochondrion. While each cell contains many mitochondria, the total mitochondrial DNA (mtDNA) in a cell represents less than 1% of the amount of nuclear DNA.

Mitochondria provide direct and indirect biochemical regulation of a wide array of cellular respiratory, oxidative and metabolic processes, including electron transport chain activity, which creates energy through the transfer of electrons derived from substrates (originating from carbohydrate, lipid and amino acids) to oxygen, which with the addition of hydrogen results in the generation of water. The transfer of electrons through the specific components of the electron transport chain also drives the transfer of protons from the mitochondrial matrix into the intermembrane space, which generates a proton gradient. This proton gradient is then harnessed to drive the production of metabolic energy in the form of adenosine triphosphate (ATP).

The mitochondrial matrix contains a complex mixture of soluble enzymes that catalyze metabolism of pyruvic acid and other small organic molecules. Pyruvic acid is oxidized by NAD⁺ producing NADH+H⁺, and then decarboxylated producing a molecule of carbon dioxide (CO₂) and a 2-carbon fragment of acetate bound to coenzyme A forming acetyl-CoA. In the citric acid cycle, this 2-carbon fragment is donated to a molecule of oxaloacetic acid. The resulting citric acid molecule (which gives its name to the process, the Citric acid cycle) undergoes a series of enzymatic steps. The final step regenerates a molecule of oxaloacetic acid and the cycle is ready to turn again. In summary, each of the 3 carbon atoms present in the pyruvate that entered the mitochondrion leaves as a molecule of carbon dioxide (CO₂). At 4 steps, a pair of electrons (2e⁻) is removed and transferred to NAD⁺ reducing it to NADH+H⁺. At one step, a pair of electrons is removed from succinic acid and reduces FAD to FADH₂. The electrons of NADH and FADH₂ are transferred to the respiratory chain, i.e., the electron transport chain.

This bioenergetic pathway consists of five enzyme complexes: NADH:CoQ oxidoreductase (Complex I, also referred to as NADH dehydrogenase), succinate:CoQ oxidoreductase (Complex II), CoQ:cytochrome c oxidoreductase (Complex III, also known as the cytochrome b-c₁ complex), cytochrome c oxidase (Complex IV, also referred to as COX) and H+-ATPase (Complex V, also known as F₀F₁-ATP synthetase, or simply ATP synthase). Both the nuclear and mitochondrial genomes are necessary for assembly of the oxidative phosphorylation enzyme complexes I, III, IV and V, while complex II is exclusively nuclear encoded. See, e.g., von Kleist-Retzow, et al., “Mitochondrial diseases—an expanding spectrum of disorders and affected genes,” Experimental Physiology 88(1):155-166 (2003). The five enzymatic complexes I-V in the mitochondrial respiratory chain consist of 80 peptides. Two freely-diffusible molecules, ubiquinone (Coenzyme Q, or CoQ) and cytochrome c, shuttle electrons from one complex to the next. CoQ shuttles electrons from Complex I and II to Complex III, and cytochrome c shuttles electrons from Complex III to Complex IV.

The respiratory chain accomplishes the stepwise transfer of electrons from NADH (and FADH₂) to oxygen molecules to form (with the aid of protons) water molecules (H₂O). Cytochrome c can only transfer one electron at a time, so cytochrome c oxidase must wait until it has accumulated 4 electrons before it can react with oxygen. The respiratory chain also harnesses energy released by this transfer to pump protons (H⁺) from the matrix to the intermembrane space. It is currently thought that approximately 20 protons are pumped into the intermembrane space per 4 electrons in order to reduce oxygen to water. Therefore a proton gradient is formed across the inner membrane by active transport and in essence forms a miniature battery. Protons can flow back down this gradient, reentering the matrix, through three routes. The first and predominant route is through the ATP synthase complex, and it is here that ATP is formed.

The second, yet not insignificant route is via a family of uncoupling proteins (UCP). To date the UCP1, UCP2 and UCP3 have been studied. These proteins appear to be involved in futile cycling of protons from the intermembrane space to the matrix, and are responsible for non-shivering derived body heat in birds and mammals. UCPs also leak protons through the membrane when there is too much energy passing through the ETC, which can generate reactive oxygen species (ROS). Therefore UCPs can protect the mitochondria from ROS, and expression of UCPs has been documented to increase in diseases associated with oxidative damage.

The third route is via direct leakage through the inner membrane lipids. Proton leakage rate is dependent on the lipid constituents of the membrane and the degree of saturation of the lipids. Reduced lipid saturation can make the membrane more permeable (leaky). Damage to the membranes may also increase proton leakage through the mitochondrial membranes.

The energy released as electrons pass down the gradient from NADH to oxygen is harnessed by three enzyme complexes of the respiratory chain (I, III, and IV) to pump protons (H⁺) against their concentration gradient from the matrix of the mitochondrion into the intermembrane space. As their concentration increases in the intermembrane space, a strong diffusion gradient is set up. As explained above, these protons can re-enter the matrix through the ATP synthase complex. The energy released as these protons flow down their electrochemical gradient is harnessed to the synthesis of ATP. This process is called chemiosmosis and is an example of facilitated diffusion.

The combined result of respiratory (oxidative) steps and the ATP-creation (phosphorylation of ADP) step is known as oxidative phosphorylation. In addition to their role in metabolic processes, among other things, mitochondria are also involved in genetically programmed cell death, i.e., “apoptosis.”

Mitochondria are demarcated from the surrounding cytosol by two sets of membranes: an inner membrane that encloses the mitochondrial matrix and an outer membrane that surrounds the inner membrane and makes out the outer border of the organelle. The space between the two membranes is termed the intermembraneous space. Protein complexes I, II, III and IV are attached to the inner wall of the inner membrane. Complex V is also found in the inner membrane. Each of the 13 proteins coded for by the mtDNA strand are all transmembrane subunits of Complex I, III, IV or V. The other proteins/enzymes required for oxidative phosphorylation—and all of the enzymes required for mtDNA replication, mtDNA repair and general mitochondrial biosynthesis—are coded for in the nucleus. Complex II is entirely coded by nuclear DNA.

The inner mitochondrial membrane is relatively impermeable to H⁺ ions (“protons”), functioning much like a hydroelectric dam, and the membrane potential of the mitochondrial membrane is nearly twice as great as that of a large nerve fiber. As noted, the respiratory enzymes, Complexes I, III and IV, pump protons out of the inner mitochondrial matrix, building proton pressure outside the “dam” (i.e., the membrane). Complex V is the “hydroelectric turbine” that utilizes the energy of the proton flow into the matrix through the “turbine” to synthesize ATP.

The specific activity of Complex I declines with age more rapidly than Complex II, which is an alternate entrance to the respiratory chain. Cytochrome-c oxidase (Complex IV) specific activity also declines with age and can result in increased production of superoxide and hydrogen peroxide. These free radicals damage the mitochondrial inner membrane, creating a positive feedback-loop for increased free-radical creation, including superoxide and hydroxyl radicals.

Superoxide (.O₂⁻) ions are generated in large numbers in mitochondria and are enzymatically converted to hydrogen peroxide (H₂O₂). The hydroxyl radical (.OH) is typically formed by oxidation of a reduced heavy metal ion (usually Fe⁺⁺ or Cu⁺) by hydrogen peroxide:

Fe⁺⁺+H₂O₂->Fe⁺⁺⁺+.OH+:OH⁻

This reaction, known as the “Fenton Reaction,” may be the most dangerous because it can occur in the cell nucleus and lead to DNA damage.

The oxidized iron (Fe⁺⁺⁺) can then catalyze the “Haber-Weiss Reaction” between superoxide and hydrogen peroxide to produce more hydroxyl radicals:

.O₂⁻+H₂O₂+O₂.OH+:OH⁻

At neutral pH the Haber-Weiss reaction occurs only to a negligible extent when no metal ion is available to act as a catalyst. In the human body nearly all iron and copper ions are tightly bound to carrier proteins (the most abundant being transferrin for iron and ceruloplasmin for copper ions. Metal ions can also react with ascorbate (vitamin C) to produce singlet oxygen (¹O₂) from normal triplet oxygen (³O₂). Wherever it is produced, the hydroxyl radical is highly reactive and can cause covalent cross-linking or free-radical propagation in a wide variety of biological molecules.

Superoxide ions tend to be concentrated in the mitochondria because they are too reactive to travel very far in an unaltered state, and are found much less frequently in the nucleus than in the cytoplasm. Similarly, hydroxyl radicals (which have a billionth-of-a-second half-life) do not drift far from their site of formation. But hydrogen peroxide molecules are more stable and can diffuse across the nuclear membrane into the nucleus or near cell membranes where hydroxyl radicals can be generated when heavy metal ions are encountered. Hydrogen peroxide can damage proteins directly by the oxidation of —SH groups.

The hydroxyl radical can react with molecules (LH) in membranes to produce lipid molecule radicals (alkyl=L)

.OH+LH->.L+H₂O

These lipid radicals can then react directly with oxygen (autoxidation) in a self-propagating chain reaction forming lipid peroxides (lipid peroxyl radicals, lipid molecules containing paired-oxygen groups —OO—):

.L+O₂->LOO.

LOO.+LH->LOOH+.L

The first reaction is about fifteen hundred times faster with singlet oxygen (¹O₂) than with normal triplet oxygen (³O₂). Singlet oxygen is energetic enough, however, that it can react directly with the double bonds of unsaturated fatty acids, without requiring a free radical intermediate.

The lipid hydroperoxides (LOOH) can promote a Fenton reaction:

Fe⁺⁺+LOOH+H⁺->Fe⁺⁺⁺+.OL+H₂O

The lipid alkoxyl radical (alkoxy=alkoxyl=.OL) is more reactive and damaging than the lipid peroxide (peroxyl) radical (peroxy=peroxyl=LOO.). Thus, by a small sequence of steps one free-radical (.L) has become two radicals (.L and .OL)—conditions for an auto-amplifying chain reaction. Nonetheless, if two alkyl, alkoxyl or peroxyl radical molecules collide they will nullify each other, but at the cost of creating a cross-link (covalent bond) between the two lipids.

The reactivity of free radicals can be quantified by a table of half-life values at 37° C. (body temperature). Short half-life corresponds to high reactivity. The one nanosecond half-life of the hydroxyl radical indicates that it is so reactive that it reacts with the first molecule it encounters.

Outside of the mitochondria, superoxide and hydrogen peroxide can be generated on the endoplasmic reticulum through oxidation processes involving cytochrome P-450 and NADPH-cytochrome c reductase. Abnormal accumulation of normal metabolites such as lactate, pyruvate, acetoacetyl-CoA and glyceraldehyde-3-phosphate can abnormally increase levels of NADH oxidase and reduced flavoenzymes such as xanthine oxidase. In the absence of sufficient electron acceptor substrates these enzymes can directly transfer electrons to O₂ or Fe⁺⁺⁺ to form superoxide or Fe⁺⁺. Ascorbate forms H₂O₂ on autoxidation (direct combination with oxygen). Both ascorbate and mercaptans (thioalcohols, i.e., compounds having “—SH” groups, where sulfur is substituted for the oxygen of alcohol) are capable of reducing Fe⁺⁺⁺ and Cu⁺⁺ to Fe⁺⁺ and Cu⁺, thereby promoting Fenton reactions.

Lipid peroxidation of polyunsaturated fatty acids exposed to oxygen leads to rancidity in foods. In living animal cells peroxidized membranes lose their permeability, becoming rigid, reactive and nonfunctional. Lipid peroxidation can produce singlet oxygen, hydroperoxides and lipid epoxides. In addition, many damaging aldehydes are formed during lipid peroxidation, particularly malondialdehyde (MDA, propanedial) and 4-hydroxynonenal (4-HNE). MDA is a major metabolite of arachidonic acid (20:4). 4-HNE is also a product of 20:4 fatty acid autoxidation, and reacts with cellular components more strongly than MDA.

Unlike free-radicals, the aldehydes MDA, 4-HNE and others are rather long-lived and can drift far from membranes, damaging a wide variety of proteins, lipids and nucleic acids. Free Radical Biology and Medicine 11:81-128 (1991). 4-HNE inactivates glucose-6-phosphate dehydrogenase, an enzyme required for the formation of NADPH and for forming ribose residues for nucleic acid biosynthesis. Aldehyde-bridge formation leads to the protein-protein cross-linking associated with lipofuscin formation.

Polyunsaturated fatty acids are more vulnerable to free radical oxidation than any other macromolecules in the body and the sensitivity to free radical damage increases exponentially with the number of double bonds. Studies of the liver lipids of mammals and a bird (pigeon) show an inverse relationship between maximum lifespan and number of double bonds. Journal of Gerontology 55A (6):B286-B291 (2000).

Animal cells contain three important enzymes to deal with the superoxide and hydrogen peroxide: catalase (CAT), glutathione peroxidase, and superoxide dismutase (SOD). Catalase catalyzes the formation of water and free oxygen from hydrogen peroxide. CAT is present in membrane-limited organelles known as peroxisomes. Peroxisomes contain enzymes that degrade amino acids and fatty acids, producing hydrogen peroxide as a byproduct.

Glutathione is a tripeptide composed of the amino acids cysteine, glycine and glutamic acid. Glutathione is the major antioxidant in the non-lipid portion of cells (most of the cytoplasm). Gutathione exists in a reduced form (GSH) and an oxidized form (GSSG). Glutathione peroxidase neutralizes hydrogen peroxide by taking hydrogens from two GSH molecules, resulting in two H₂O and one GSSG. The enzyme glutathione reductase then regenerates GSH from GSSG with NADPH as a source of hydrogen.

Superoxide dismutases are the most abundant anti-oxidant enzymes in animals. The liver, in particular, is very high in SOD. Dismutases are enzymes that catalyze the reaction of two identical molecules to produce molecules in different oxidative states. In the absence of SOD, two superoxide ions can spontaneously dismutate to produce hydrogen peroxide and singlet oxygen. SOD catalyzes a reaction between two superoxide ions to produce hydrogen peroxide and triplet oxygen. There are three isoforms of superoxide dismutase (SOD): cytosolic or copper-zinc SOD (CuZn-SOD), manganese SOD (Mn-SOD) localized in the mitochondrial matrix, and an extracellular form of CuZn-SOD (EC-SOD). CuZn-SOD has also been localized to the mitochondrial intermembrane space.

Cellular concentration of SOD relative to metabolic activity is a lifespan predictor for animal species. Most mammals experience a lifetime energy expenditure of about 200,000 calories per gram, while humans have an energy expenditure of about 800,000 calories per gram. Humans also have the highest levels of SOD (relative to metabolic rate) of all mammal species studied. But in absolute terms maximum lifespan correlates negatively with antioxidant enzyme levels and correlates positively with a lower rate of free-radical production and higher rate of DNA repair. Journal of Comparative Physiology B168:149-158 (1998). For example, oxidative damage to DNA is ten times greater in rats than in humans. One of the reasons that mitochondria are surrounded by membranes may be to protect the cell from the free-radicals they generate. DNA may be sequestered in the nucleus, in part, as additional protection against free radicals. Nonetheless, free radicals contribute to DNA damage and mutation.

In addition to enzymes, the animal cell uses many other chemicals to protect against oxygen free-radicals. Vitamin E is the main free-radical trap in the (lipid) membranes. Vitamin C acts as an anti-oxidant in the non-lipid (watery) portions of cells, between cells and in the bloodstream. Melatonin, a hormone produced by the pineal gland in decreasing quantities with aging, efficiently crosses membranes (including the nucleus) and is effective against hydroxyl radicals.

Coenzyme Q (CoQ), also known as ubiquinone because it is ubiquitous in almost all cellular organisms, with the exception of gram-positive bacteria and some fungi, is an essential component of the mitochondrial respiratory chain. CoQ forms an important part of the antioxidant defense against superoxide radicals. Both Complex I and Complex II dehydrogenase can reduce CoQ to CoQH₂, which is subsequently oxidized in two steps—first to .CoQ⁻, and then to CoQ. However, .CoQ⁻ is unstable and can errantly transfer an electron to an O₂ molecule resulting in superoxide ion (.O₂⁻) formation.

Free radical damage in the cell may be caused, in part, by mitochondrial “leaking”. Damaged or defective mitochondria may leak, for example, protons, and relatively stable fee radicals. The most damaged mitochondria are consumed by lysosomes, while defective mitochondria (which produce less ATP as well as less superoxide) remain to reproduce themselves. Rejuvenation Research 8(1):3-9 (2005).

An estimated 1-5% of oxygen used by mitochondria will normally “leak” from the respiratory chain to form superoxide. Journal of Neurochemistry 59:1609-1623 (1992); Journal Of Internal Medicine 238:405-421 (1995).

Increasing insulin levels associated with aging and type-2 diabetes stimulate nitric oxide synthetase resulting in peroxynitrite. International Journal of Biochemistry and Cell Biology 34:1340-1354 (2002). Lipid peroxidation of the inner mitochondrial membrane by peroxynitrite can increase proton leak independent of UCPs. Peroxynitrite can also degrade the function of respiratory enzymes (Journal of Neurochemistry 70:2195-2202 (1998)) and inactivate mitochondrial superoxide dismutase (Mn-SOD) enzyme (Proc. Nat. Acad Sci. (USA) 93(21)11853-11858 (1996)).

The Mn-SOD of mitochondria can be induced to higher concentrations by oxidative stress (in contrast to the cytoplasmic Cu/Zn-SOD which is constitutive rather than induced). A comparison of seven non-primate mammals (mouse, hamster, rat, guinea-pig, rabbit, pig and cow) showed that the rate of mitochondrial superoxide and hydrogen peroxide production in heart and kidney were inversely correlated with maximum life span. Free Radical Biology and Medicine 15:621-627 (1993).

Aging is associated with decreased oxidative phosphorylation, coupling efficiency and increased superoxide production. Mitochondria of older organisms are fewer in number, larger in size and less efficient (produce less ATP and more superoxide).

A comparison of the heart mitochondria in rats (4-year lifespan) and pigeons (35-year lifespan) showed that pigeon mitochondria leak fewer free-radicals than rat mitochondria, despite the fact that both animals have similar metabolic rate and cardiac output. Pigeon heart mitochondria (Complexes I and III) showed a 4.6% free radical leak compared to a 16% free radical leak in rat heart mitochondria. Mechanisms of Aging and Development 98:95-111 (1997). Hummingbirds use thousands of calories in a day (more than most humans) and have relatively long lifespans (the broad-tailed hummingbird Selasphorus platycerus reportedly has a maximum lifespan in excess of 8 years). Birds have more saturated lipid (and therefore reduced oxidizability) in their mitochondrial membranes and have higher levels of small-molecule antioxidants, such as ascorbate and uric acid.

The damage to cellular proteins, lipids and DNA throughout the cell from free-radicals generated by mitochondria has also been implicated as a cause of aging. If fatty acids entering mitochondria for energy-yielding oxidation have been peroxidized in the blood, this places an additional burden on antioxidant defenses. The greatest damage occurs in the mitochondria themselves, including damage to the respiratory chain protein complexes (leading to higher levels of superoxide production), damage to the mitochondrial membrane (leading to membrane leakage of calcium ions and other substances) and damage to mitochondrial DNA (leading to further damage to mitochondrial protein complexes). Improvement of mitochondrial encoded protein synthesis fidelity in yeast demonstrated a 27% increase in mean life span. Journal of Gerontology 57A(1):B29-B36 (2002).

mtDNA deletion mutations have also been reported to accumulate in post-mitotic cells with age. Biochimica et Biophysica Acta 410:183-193 (1999). The mitochondrial theory of aging postulates that damage to mtDNA and organelles by free radicals leads to loss of mitochondrial function and loss of cellular energy (with loss of cellular function). Mutations in mtDNA occur at 16-times the rate seen in nuclear DNA. Unlike nuclear DNA, mtDNA has no protective histone proteins, and DNA repair is less efficient in mitochondria than in the nucleus. These factors may account for more rapid aging seen with Complex I and III as compared to Complex II and IV. Aging mitochondria become enlarged and, if they can be engulfed by lysosomes, are resistant to degradation and contribute to lipofuscin formation. European J Biochemistry 269(8):1996-2002 (2002). Also associated with aging is a decline in the amount of CoQ in organs. Declines in functional mitochondria and CoQ10 with age is most damaging to those organs that have the highest energy demands per gram of tissue, namely, the heart, kidney, brain, liver and skeletal muscle, in that order. Journal of Internal Medicine 238:405-421 (1995). Clinically, damage to brain and muscle tissue are the first symptoms of mitochondrial disease. Therapy has included the B-vitamins that act as coenzymes in the respiratory chain (thiamine, riboflavin, niacinamide) and CoQ10. Acta Neurologica Scandinavia 92:273-280 (1995).

According to generally accepted theories of mitochondrial function, proper respiratory activity requires maintenance of an electrochemical potential in the inner mitochondrial membrane by a coupled chemiosmotic mechanism. Conditions that dissipate or collapse this membrane potential, including but not limited to failure at any step of the electron transport chain may prevent ATP biosynthesis. Altered or defective mitochondrial activity may also result in a catastrophic mitochondrial collapse that has been termed “mitochondrial permeability transition” (MPT) during which a large pore complex spanning through both mitochondrial membranes is opened.

In addition, mitochondrial proteins such as cytochrome c and “apoptosis inducing factor” may dissociate or be released from mitochondria due to MPT (or the action of mitochondrial proteins such as Bax), and may induce proteases known as caspases and/or stimulate other events in apoptosis. Drug Dev. Res. 46:18-25, 1999. Cytochrome C is reported to combine with apoptosome, activating factor 1 (Apaf-1), procaspase-9 and dATP to form the apoptosome, a multimeric complex which activates caspase-9, which in turn activates downstream caspases leading to cleavage of apoptotic targets.

As noted, defective mitochondrial activity may also result in the generation of highly reactive free radicals that have the potential of damaging cells and tissues. Oxygen free radical induced lipid peroxidation, for example, is a well established pathogenetic mechanism in central nervous system injury, such as that found in a number of degenerative diseases and in ischemia (i.e., stroke). Mitochondrial participation in the apoptotic cascade is believed to also be a key event in the pathogenesis of neuronal death.

There are at least two deleterious consequences of exposure to reactive free radicals arising from mitochondrial dysfunction that adversely impact the mitochondria themselves. First, free radical mediated damage may inactivate one or more electron transport chain proteins. Second, free radical mediated damage may result in MPT. According to generally accepted theories of mitochondrial function, proper electron transport chain respiratory activity requires maintenance of an electrochemical potential in the inner mitochondrial membrane by a coupled chemiosmotic mechanism. Free radical oxidative activity may dissipate this membrane potential, thereby preventing ATP biosynthesis and/or triggering mitochondrial events in the apoptotic cascade.

For example, rapid mitochondrial permeability transition likely entails changes in the inner mitochondrial transmembrane protein adenylate translocase that results in the formation of a “pore” (the MTP pore mentioned above). Whether this pore is a distinct conduit or simply a widespread leakiness in the membrane is unresolved. In any event, because membrane permeability transition is potentiated by free radical exposure, it may be more likely to occur in the mitochondria of cells from patients having mitochondria associated diseases that are chronically exposed to such reactive free radicals.

In sum, defective mitochondrial activity, including but not limited to failure at any step of the electron transport chain, may result in (i) decreases in ATP production, (ii) increases in the generation of highly reactive free radicals (e.g., superoxide, peroxynitrite and hydroxyl radicals, and hydrogen peroxide), (iii) disturbances in intracellular calcium homeostasis and (iv) the release of factors (such as cytochrome c and “apoptosis inducing factor”) that initiate or stimulate the apoptosis cascade. Because of these biochemical changes, mitochondrial dysfunction has the potential to cause widespread damage to cells and tissues.

A number of diseases and disorders are thought to be caused by or be associated with alterations in mitochondrial metabolism and/or inappropriate induction of mitochondria-related functions leading to apoptosis. These include, by way of example and not limitation, auto-immune disease, Alpers Disease (progressive infantile poliodystrophy, Barth syndrome, congenital muscular dystrophy, fatal infantile myopathy, “later-onset” myopathy, MELAS (mitochondrial encephalopathy, lactic acidosis, and stroke), MIDD (mitochondrial diabetes and deafness), MERRF (myoclonic epilepsy ragged red fiber syndrome), arthritis, NARP (Neuropathy; Ataxia; Retinitis Pigmentosa), MNGIE (Myopathy and external ophthalmoplegia; Neuropathy; Gastro-Intestinal; Encephalopathy), LHON (Leber's; Hereditary; Optic; Neuropathy), Kearns-Sayre disease, Pearson's Syndrome, PEO (Progressive External Ophthalmoplegia), Wolfram syndrome, DIDMOAD (Diabetes Insipidus, Diabetes Mellitus, Optic Atrophy, Deafness), ADPD (Alzheimer's disease; Parkinson's disease), AMFD (ataxia, myoclonus and deafness), CIPO (chronic intestinal pseudoobstruction; myopathy; opthalmoplegia), CPEO (chronic progressive external opthalmoplegia), maternally inherited deafness, aminoglycoside-induced deafness, DEMCHO (dementia; chorea), DMDF (diabetes mellitus; deafness), exercise intolerance, ESOC (epilepsy; strokes; optic atrophy; congenitive decline), FBSN (familial bilateral striatal necrosis), FICP (fatal infantile cardiomyopathy plus a MELAS-associated cardiomyopathy), GER (gastrointestinal reflux), LCHAD (Long-Chain Hydroxyacyl-CoA Dehydrogenase Deficiency), SCHAD (Sharot-Chain Hydroxyacyl-CoA Dehydrogenase Deficiency), MAD (Multiple Acyl-CoA Dehydrogenase Deficiency) MCAD (Medium-Chain Acyl-CoA Dehydrogenase Deficiency), SCAD (Short-Chain Acyl-CoA Dehydrogenase Deficiency), VLCAD (very long-chain Acyl-CoA Dehydrogenase Deficiency), LIMM (lethal infantile mitochondrial myopathy), LDYT (Leber's hereditary optic neuropathy and DYsTonia), Luft Disease, MDM (myopathy; diabetes mellitus), MEPR (myoclonic epilepsy; psychomotor regression), MERME (MERRF/MELAS overlap disease), MHCM (maternally inherited hypertrophic cardiomyopathy), MICM (maternally inherited cardiomyopathy), MILS (maternally inherited Leigh syndrome), mitochondrial encephalocardiomyopathy, mitochondrial encephalomyopathy, mitochondrial myopathy, MMC (maternal myopathy; cardio myopathy), multisystem mitochondrial disorder (myopathy; encephalopathy; blindness; hearing loss; peripheral neuropathy), NIDDM (non-insulin dependent diabetes mellitus), Pearson Syndrome PEM (progressive encephalopathy), PME (progressive myclonus epilepsy), Rett syndrome, SIDS (sudden infant death syndrome, SNHL (sensorineural hearing loss), Leigh's Syndrome, dystonia, schizophrenia, and psoriasis.

Altered mitochondrial function characteristic of the mitochondria associated diseases may also be related to loss of mitochondrial membrane electrochemical potential by mechanisms other than free radical oxidation. Such transition permeability may result from direct or indirect effects of mitochondrial genes, gene products or related downstream mediator molecules and/or extra-mitochondrial genes, gene products or related downstream mediators, or from other known or unknown causes. Loss of mitochondrial potential therefore may be a critical event in the progression of mitochondria associated or degenerative diseases.

Various mitochondrial disorders result from partial dysfunction of mitochondrial oxidative phosphorylation. Respiratory chain disorders include Complex I: NADH dehydrogenase (NADH-CoQ reductase) deficiency, Complex II: Succinate dehydrogenase deficiency, Complex III: Ubiquinone-cytochrome c oxidoreductase deficiency, Complex IV: Cytochrome c oxidase (COX) deficiency, and Complex V: ATP synthase deficiency. See, e.g., Smeitink, J A, “Mitochondrial disorders: clinical presentation and diagnostic dilemmas,” J. Inherit. Metab. Dis. 2003; 26(2-3):199-207; Szewczyk, A., Wojtczak, L., “Mitochondria as a pharmacological target,” Pharmacol Rev. 2002 March; 54(1):101-27; Orth, M., and Schapira, A. H., “Mitochondria and degenerative disorders,” Am J Med Genet. 2001 Spring;106(1):27-36; Cottrell, D. A., and Turnbull, D. M., “Mitochondria and ageing,” Curr. Opin. Clin. Nutr. Metab. Care. 2000 November; 3(6):473-8; Angelini, C., “Hypertrophic cardiomyopathy with mitochondrial myopathy. A new phenotype of complex II defect,” Japanese Heart Journal 1993, 34(1), 63-77; Antozzi, C., “Epilepsia partialis continua associated with NADH-coenzyme Q reductase deficiency,” J. Neurol. Sci., 1995, 129(2), 152-161; Bentlage, H A, “Lethal infantile mitochondrial disease with isolated complex I deficiency in fibroblasts but with combined complex I and IV deficiencies in muscle,” Neurology, 1996, 47(1), 243-248; Berio, A., “Marinesco-Sjogren syndrome with chronic progressive ophthalmoplegia caused by presumed defective oxidative phosphorylation,” Pediatr. Med. Chir., 1996, 18(1), 99-103; Bindoff, L A, “Multiple defects of the mitochondrial respiratory chain in a mitochondrial encephalopathy (MERRF): a clinical, biochemical and molecular study,” Journal of the Neurological Sciences, 1991, 102(1), 17-24; Boffoli, D., “Decline with age of the respiratory chain activity in human skeletal muscle,” Biochim Biophys Acta, 1994, 1226(1), 73-82; Buchwald, A., “Alterations of the mitochondrial respiratory chain in human dilated cardiomyopathy,” Eur. Heart. J., 1990, 11(6), 509-16; Byrne, E., “New concepts in respiratory chain diseases,” Current Opinion in Rheumatology, 1992, 4(6), 784-93; Campos, Y., “Respiratory chain enzyme defects in patients with idiopathic inflammatory myopathy,” Annals of the Rheumatic Diseases, 1995, 54(6), 491-3; Chalmers, R. M., “Sequence of mitochondrial DNA in patients with multiple sclerosis,” Ann. Neurol., 1996, 40(2), 239-243; Cortopassi, G., “Modelling the effects of age-related mtDNA mutation accumulation; complex I deficiency, superoxide and cell death,” Biochimica et Biophysica Acta, 1995, 1271(1), 171-6; Ernster, L., “Biochemical, physiological and medical aspects of ubiquinone function,” Biochimica et Biophysica Acta, 1995, 1271(1), 195-204; Goncalves, I., “Mitochondrial respiratory chain defect: a new etiology for neonatal cholestasis and early liver insufficiency,” J. Hepatol., 1995, 23(3), 290-294; Gu, M., “Mitochondrial respiratory chain function in multiple system atrophy,” Mov. Disord., 1997, 12(3), 418-22; Haas, R. H., “Oxidative metabolism in Rett syndrome: 2. Biochemical and molecular studies,” Neuropediatrics, 1995, 26(2), 95-9; Heddi, A., “Steady state levels of mitochondrial and nuclear oxidative phosphorylation transcripts in Kearns-Sayre syndrome,” Biochimica et Biophysica Acta, 1994, 1226(2), 206-12; Ibel, H., “Multiple respiratory chain abnormalities associated with hypertrophic cardiomyopathy and 3-methylglutaconic aciduria,” European Journal of Pediatrics, 1993, 152(8), 665-70; Majamaa, K., “Metabolic interventions against complex I deficiency in MELAS syndrome,” Mol. Cell Biochem., 1997, 174(1-2), 291-6; Maurer, I., “Coenzyme Q10 and respiratory chain enzyme activities in hypertrophied human left ventricles with aortic valve stenosis,” Am. J. Cardiol., 1990, 66(4), 504-5; Maurer, I., “Myocardial respiratory chain enzyme activities in idiopathic dilated cardiomyopathy, and comparison with those in atherosclerotic coronary artery disease and valvular aortic stenosis,” Am. J. Cardiol., 1993, 72(5), 428-33; Mierzewska, H., “Mitochondrial diseases. Part I—general review, Neurol. Neurochir. Pol., 1996, 30(2), 265-278; Muller-Hocker, J., “Defects of the respiratory chain in the normal human liver and in cirrhosis during aging,” Hepatology, 1997, 26(3), 709-19; Pitkanen, S., “Mitochondrial complex I deficiency leads to increased production of superoxide radicals and induction of superoxide dismutase,” J Clinical Investigation, 1996, 98(2), 345-351; Shoffner, J. M., “Oxidative phosphorylation diseases and stroke,” Heart Disease and Stroke, 1993, 2(5), 439-45.

Mitochondrial dysfunction is also thought to be critical in the cascade of events leading to apoptosis in various cell types. Kroemer et al., FASEB J. 9:1277-1287 (1995). Perturbation of mitochondrial respiratory activity leading to altered cellular metabolic states, such as elevated intracellular ROS, may occur in mitochondria associated diseases and may further induce pathogenetic events via apoptotic mechanisms.

Neuronal death following stroke occurs in an acute manner, and the literature documents the importance of mitochondrial function in neuronal death following ischemia/reperfusion injury that accompanies stroke, cardiac arrest and traumatic injury to the brain. Experimental support continues to accumulate for a central role of defective energy metabolism, alteration in mitochondrial function leading to increased oxygen free radical production and impaired intracellular calcium homeostasis, and active mitochondrial participation in the apoptotic cascade in the pathogenesis of acute neurodegeneration. A stroke occurs when a region of the brain loses perfusion and neurons die acutely or in a delayed manner as a result of this sudden ischemic event. Upon cessation of the blood supply to the brain, tissue ATP concentration drops to negligible levels within minutes. At the core of the infarct, lack of mitochondrial ATP production causes loss of ionic homeostasis, leading to osmotic cell lysis and necrotic death. A number of secondary changes can also contribute to cell death following the drop in mitochondrial ATP. Cell death in acute neuronal injury radiates from the center of an infarct where neurons die primarily by necrosis to the penumbra where neurons undergo apoptosis to the periphery where the tissue is still undamaged. Martin et al., Brain Res. Bull. 46:281-309 (1998).

Mitochonrial swelling and aggregation has been reported in patients with erectile dysfunction. Aydos K. et al., Int. Urol. Nephrol. 28(3):375-85 (1996). Erectile dysfunction affects 30 million men just in the United States. Treatments available for erectile dysfunction and decreased sex drive include the phsophodiesterase-5 inhibotos, for example, Viagra, Levitra and Cialis. Side effects of all three do occur and include headache, upset stomach, flushing and nasal congestion. Viagra may also cause changes in vision and Cialis may also cause back pain. In addition, many men over the age of 50 are not served by the current treatments for erectile dysfunction due to limited efficacy, side effects, and potential drug-drug interactions.

Triethylenetetramine dihydrochloride, a chelating compound for removal of excess copper from the body, is prescribed for Wilson's disease patients who cannot tolerate penicillamine. Triethylenetetramine di hydrochloride is N,N′-bis(2-aminoethyl)-1,2-ethanediamine dihydrochloride. It is a white to pale yellow crystalline hygroscopic powder. Syprine® (triethylenetetramine dihydrochloride) is available as 250 mg capsules for oral administration. See Siegemund R, et al., “Mode of action of triethylenetetramine dihydrochloride on copper metabolism in Wilson's disease,” Acta Neurol. Scand. 83(6):364-6 (June 1991).

U.S. Pat. Nos. 6,610,693, 6,348,465 and 6,951,890 provide copper chelators and other agents (e.g., zinc which prevents copper absorption) to decrease copper values for the benefit of subjects suffering from diabetes and its complications. See also, Cooper, G. J., et al., “Treatment of diabetes with copper binding compounds,” U.S. Pat. App. No. 2005/0159489, published Jul. 21, 2005; Cooper, G. J., et al., “Copper antagonist compounds,” U.S. Pat. App. No. 2005/0159364, published Jul. 21, 2005; Cooper, G. J., et al., “Preventing and/or treating cardiovascular disease and/or associated heart failure,” U.S. Pat. App. No. 2003/0203973, published Oct. 30, 2003. These also relate to therapies using copper antagonists, including triethylenetetramine, for example. Various experimental and clinical results are described in Cooper, G. J., et al., “Regeneration of the heart in diabetes mellitus by selective copper chelation,” Diabetes 53:2501-2508 (2004). See also Cooper. G. J., et al., “Demonstration of a Hyperglycemia-Driven Pathogenic Abnormality of Copper Homeostasis in Diabetes and Its Reversability by Selective Chelation: Quantitative Comparisons Between the Biology of Copper and Eight Other Nutritionally Essential Elements in Normal and Diabetic Subjects,” Diabetes 54:1468-1476 (2005).

Current treatment for mitochondrial related disease and aging are directed to treating the symptoms of these diseases, disorders and conditions. There are no known approved treatments that are directed to the underlying mitochondrial dysfunction and the resulting cell and tissue damage. Clearly there is a need for compounds and methods that limit or prevent damage to mitochondria, as well as damage to organelles, cells and tissues by free radicals generated intracellularly as a direct or indirect result of mitochondrial dysfunction. Drugs relating to the alteration of mitochondrial function have great potential for a broad based therapeutic strategy for related diseases. Depending on the disease or disorder for which treatment is sought, such drugs may be mitochondria protecting agents or anti-apoptotic agents.

There is also a need for compounds and methods that limit or prevent damage to cells and tissues that occurs directly or indirectly as a result of necrosis and/or inappropriate apoptosis. In particular, because mitochondria are mediators of apoptotic events, agents that modulate mitochondrially mediated pro-apoptotic events would be especially useful. Such agents may be suitable for the treatment of acute events such as stroke and infarct, for example. Agents and methods that maintain mitochondrial integrity represent novel protective agents with utility in limiting mitochondrial and mitochondria-related injury.

The present inventions fulfill these needs and provide other related advantages. Those skilled in the art will recognize further advantages and benefits of the invention after reading the disclosure.

### SUMMARY OF THE INVENTION

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

It has been discovered that certain compounds, including those described or referenced herein, can mitigate mitochondrial swelling, elevated mitochondrial protein expression, and elevated expression of nuclear mitochondrial genes.

It has also been discovered that certain compounds, including those described or referenced herein, can lessen elevated mitochondria number.

Furthermore, it has been discovered that certain compounds, including those described or referenced herein, can assist in lowering elevated TGF-β1 levels.

Additionally, it has been discovered that certain compounds, including those described or referenced herein, can assist in normalizing lowered Cu⁺¹ levels.

Additionally, it has been discovered that certain compounds, including those described or referenced herein, can assist in normalizing Smad 4 levels.

It has been discovered that certain compounds, including those described or referenced herein, can assist in normalizing collagen IV levels.

It has been discovered that certain compounds, including those described or referenced herein, can mitigate and/or normalize pathological abnormalities in the electron transport chain (ETC) complexes in the mitochondria.

The present inventions relate generally to compounds, compositions and methods for treating mitochondria-associated diseases, including respiratory chain disorders. The inventions also relate to diseases and disorders in which free radical mediated oxidative injury leads to tissue degeneration, and diseases and disorders in which cells inappropriately undergo programmed cell death (apoptosis), leading to tissue degeneration.

The present inventions also relate to compositions and methods for treating such disease and disorders through the use of compounds which function as, respectively, mitochondria protecting agents, mitochondria biogenesis agents, and anti-apoptotic agents.

The present inventions are directed in part to the treatment of mitochondria-associated diseases by administration to a mammal in need thereof an effective amount of a copper binding tetramine compound, particularly tetramine compounds that bind Cu⁺², and preferably tetramine compounds that are specific for Cu⁺² over Cu⁺¹. Tetramine compounds include triethylenetetramine (2,2,2 tetramine), 2,3,2 tetramine and 3,3,3 tetramine as well as salts, active metabolites, derivatives, and prodrugs thereof.

The present inventions are also directed in part to the treatment of mitochondria-associated diseases by administration to a mammal in need thereof an effective amount of a compound according to Formula (I) or Formula (II).

In still further embodiments, methods are provided for treating mitochondria-associated diseases by administering one or more copper binding tetramine compounds, compounds of Formula (I), or compounds of Formula (II), in the form of a pharmaceutical composition. Thus, pharmaceutical compositions are also provided comprising one or more copper binding tetramine compounds, compounds of Formula (I), or compounds of Formula (II), in combination with a pharmaceutically acceptable carrier or diluent.

In the context of the inventions, mitochondria-associated diseases include diseases in which free radical mediated oxidative injury leads to tissue degeneration, and diseases in which cells inappropriately undergo apoptosis, and include the treatment of a wide number of mitochondria-associated diseases, including but not limited to auto-immune disease, congenital muscular dystrophy, fatal infantile myopathy, “later-onset” myopathy, MELAS (mitochondrial encephalopathy, lactic acidosis, and stroke), MIDD (mitochondrial diabetes and deafness), MERRF (myoclonic epilepsy ragged red fiber syndrome), arthritis, NARP (Neuropathy; Ataxia; Retinitis Pigmentosa), MNGIE (Myopathy and external ophthalmoplegia; Neuropathy; Gastro-Intestinal; Encephalopathy), LHON (Leber's; Hereditary; Optic; Neuropathy), Kearns-Sayre disease, Pearson's Syndrome, PEO (Progressive External Ophthalmoplegia), Wolfram syndrome, DIDMOAD (Diabetes Insipidus, Diabetes Mellitus, Optic Atrophy, Deafness), ADPD (Alzheimer's disease; Parkinson's disease), AMFD (ataxia, myoclonus and deafness), CIPO (chronic intestinal pseudoobstruction; myopathy; opthalmoplegia), CPEO (chronic progressive external opthalmoplegia), maternally inherited deafness, aminoglycoside-induced deafness, DEMCHO (dementia; chorea), DMDF (diabetes mellitus; deafness), exercise intolerance, ESOC (epilepsy; strokes; optic atrophy; congenitive decline), FBSN (familial bilateral striatal necrosis), FICP (fatal infantile cardiomyopathy plus a MELAS-associated cardiomyopathy), GER (gastrointestinal reflux), LIMM (lethal infantile mitochondrial myopathy), LDYT (Leber's hereditary optic neuropathy and DYsTonia), MDM (myopathy; diabetes mellitus), MEPR (myoclonic epilepsy; psychomotor regression), MERME (MERRF/MELAS overlap disease), MHCM (maternally inherited hypertrophic cardiomyopathy), MICM (maternally inherited cardiomyopathy), MILS (maternally inherited Leigh syndrome), mitochondrial encephalocardiomyopathy, mitochondrial encephalomyopathy, mitochondrial myopathy, MMC (maternal myopathy; cardio myopathy), multisystem mitochondrial disorder (myopathy; encephalopathy; blindness; hearing loss; peripheral neuropathy), NIDDM (non-insulin dependent diabetes mellitus), PEM (progressive encephalopathy), PME (progressive myclonus epilepsy), Rett syndrome, SIDS (sudden infant death syndrome, SNHL (sensorineural hearing loss), Leigh's Syndrome, dystonia, schizophrenia, and psoriasis.

For example, the inventions concern the use of therapeutic agents having utility for regulating increased mitochondria number in vivo, as well as pharmaceutical compositions containing such agents, articles and kits and delivery devices containing such agents, and tablets and capsules and formulations comprising such agents or compositions.

The inventions also concern the use of therapeutic agents having utility for regulating increased mitochondria mass in vivo, as well as pharmaceutical compositions containing such agents, articles and kits and delivery devices containing such agents, and tablets and capsules and formulations comprising such agents or compositions.

The inventions also concern the use of therapeutic agents having utility for regulating increased mitochondria protein expression in vivo, as well as pharmaceutical compositions containing such agents, articles and kits and delivery devices containing such agents, and tablets and capsules and formulations comprising such agents or compositions.

The inventions also concern the use of therapeutic agents having utility for regulating mitochondrial swelling in vivo, as well as pharmaceutical compositions containing such agents, articles and kits and delivery devices containing such agents, and tablets and capsules and formulations comprising such agents or compositions.

The inventions also concern the use of therapeutic agents having utility for regulating increased expression nuclear mitochondria genes in vivo, of as well as pharmaceutical compositions containing such agents, articles and kits and delivery devices containing such agents, and tablets and capsules and formulations comprising such agents or compositions.

The inventions also concern the use of therapeutic agents having utility for regulating increased TGFβ-1 expression in vivo, as well as pharmaceutical compositions containing such agents, articles and kits and delivery devices containing such agents, and tablets and capsules and formulations comprising such agents or compositions.

The inventions also concern the use of therapeutic agents having utility for elevating depressed copper (I) levels (Cu⁺¹ levels) in vivo, as well as pharmaceutical compositions containing such agents, articles and kits and delivery devices containing such agents, and tablets and capsules and formulations comprising such agents or compositions.

The inventions also concern the use of therapeutic agents having utility for regulating increased Smad 4 expression in vivo, as well as pharmaceutical compositions containing such agents, articles and kits and delivery devices containing such agents, and tablets and capsules and formulations comprising such agents or compositions.

The inventions also concern the use of therapeutic agents having utility for regulating increased collagen IV expression in vivo, as well as pharmaceutical compositions containing such agents, articles and kits and delivery devices containing such agents, and tablets and capsules and formulations comprising such agents or compositions.

The inventions also concern the use of therapeutic agents having utility for regulating increased cytochrome c release from mitochonria in vivo, as well as pharmaceutical compositions containing such agents, articles and kits and delivery devices containing such agents, and tablets and capsules and formulations comprising such agents or compositions.

The inventions also concern the use of therapeutic agents, i.e., copper antagonists, having utility for increasing cytochrome c oxidase activity in vivo, as well as pharmaceutical compositions containing such agents, articles and kits and delivery devices containing such agents, and tablets and capsules and formulations comprising such agents or compositions, all of which are provided herein.

The inventions also concern the use of therapeutic agents having utility for treating erectile dysfuntion, as well as pharmaceutical compositions containing such agents, articles and kits and delivery devices containing such agents, and tablets and capsules and formulations comprising such agents or compositions.

Pharmaceutical compositions also comprise a pharmaceutically acceptable carrier or diluent.

The patent is also directed to methods for assaying or screening for agents or suspected agents having utility in the regulation of mitochondria number, regulating mitochondria mass, regulating mitochondria protein expression, regulating nuclear mitochondria gene expression, regulating TGFβ-1 expression, and/or regulating Cu⁺¹ levels using methods described and claimed herein.

Useful compounds include pharmaceutically acceptable polyamines, including copper-binding polyamines. Polyamines may include, for example, spermidine, as well as spermine and other tetramines. Tetramines also include, for example, triethylenetetramine (2,2,2 tetramine), as well as salts, active metabolites, derivatives, and prodrugs thereof. Salts include, for example, triethylenetetramine hydrochloride salts (e.g., triethylenetetramine dihydrochloride) and succinate salts (e.g., triethylenetetramine disuccinate), as well as maleate salts (e.g., triethylenetetramine tetramaleate) and fumarate salts (e.g., triethylenetetramine tetrafumarate). Metabolites include, for example, acetylated metabolites, such as N-acetyl triethylenetetramine (e.g., monoacetyl-triethylenetetramine). Derivatives include, for example, PEG-modified tetramines, including PEG-modified triethylenetetramines Other useful compounds include pharmaceutically acceptable compounds of Formula I and Formula II herein. Suitable copper antagonists include, for example, penicillamine, N-methylglycine, N-acetylpenicillamine, tetrathiomolybdate, 1,8-diamino-3,6,10,13,16,19-hexa-azabicyclo[6.6.6]icosane, N,N′-diethyldithiocarbamate, bathocuproinedisulfonic acid, and bathocuprinedisulfonate.

Other suitable compounds include, for example, pharmaceutically acceptable linear or branched tetramines capable of binding copper.

The invention includes methods for treating a subject having or suspected of having or predisposed to, or at risk for, for example, any diseases, disorders and/or conditions described or referenced herein. Such compounds may be administered in amounts, for example, that are effective to (1) decrease mitochondrial number, (2) decrease mitochondrial protein expression, (3) decrease expression of nuclear mitochondrial genes, (4) decrease mitochondrial swelling, (5) decrease TGFβ-1 levels, (6) increase Cu⁺¹ levels, (7) decrease Smad 4 levels, (8) increase cytochrome c activity, (9) regulate increased cytochand/or (9) decrease collagen IV levels. Such compositions include, for example, tablets, capsules, solutions and suspensions for parenteral and oral delivery forms and formulations.

The patent is also directed to a method for assaying a drug candidate and, more specifically, to a method for measuring the activity of a drug candidate and a copper-binding tetramine, for example, and then comparing the actions of the compounds against a predetermined correlation measurement (e.g., a decrease in mitochondrial number, decreased mitochondrial protein expression, decreased expression of nuclearly encoded mitochondrial genes, decreased mitochondrial swelling, a decrease TGFβ-1 levels, an increase Cu⁺¹ levels, a decrease in Smad 4 levels and/or a decrease in collagen IV levels) to evaluate or measure at least one activity or potential activity of one or more drug candidates.

These and other aspects of the inventions, which are not limited to or by the information in this Brief Summary, are provided below.

### BRIEF DESCRIPTION OF THE DRAWINGS

FIGS. 1A-1C show the level of total ion levels calculated by PIXE analysis for the control, diabetic and triethylenetetramine dihydrochloride treated groups. FIG. 1A demonstrates statistically significant difference (P<0.05) in copper levels between control and diabetic and between diabetic and triethylenetetramine dihydrochloride treated groups. FIG. 1B demonstrates a non-statistically significant difference in Zinc levels between the control and diabetic groups and a significant difference between the diabetic and triethylenetetramine dihydrochloride treated groups. FIG. 1C demonstrates a statistically significant difference in Iron levels between control and diabetic groups and a non-statistically significant difference between the diabetic and triethylenetetramine di hydrochloride treated groups.

FIGS. 2A-2C show there is no statistically significant difference in sodium (FIG. 2A), manganese (FIG. 2B) or phospherous (FIG. 2C) levels in the control, diabetic or triethylenetetramine di hydrochloride treated groups.

FIG. 3 shows there is no statistically significant difference in sulphur (FIG. 3A), chlorine (FIG. 3B) or potassium (FIG. 3C) levels in the control, diabetic or triethylenetetramine di hydrochloride treated groups.

FIG. 4 shows there is no statistically significant difference in calcium levels in the control, diabetic or triethylenetetramine dihydrochloride treated groups

FIG. 5 shows a chart which lists the 14 proteins, from the group of 33 proteins, discovered to be significantly changed back to normal levels in T-STZ rats (p<0.05).

FIG. 6 shows a chart which lists an additional 6 proteins that were significantly altered in STZ rats.

FIGS. 7A and 7B illustrate the effects of spermine, spermidine and triethylenetetramine dihydrochloride on mitochondrial volume in diabetic (FIG. 7A) or control (FIG. 7B) mitochondria.

FIGS. 8A and 8B illustrate the change, if any, in mitochondrial volume in diabetic (FIG. 8A) or control (FIG. 8B) mitochondria exposed to spermidine, spermine or triethylenetetramine dihydrochloride after the addition of Calcium.

FIG. 9 illustrates any change in mitochondrial volume diabetic or control mitochondria exposed to triethylenetetramine dihydrochloride against a background of 5 mM spermine.

FIG. 10 compares the level of mRNA expression the 14 proteins identified in Example 2, plus 2 additional proteins.

FIGS. 11A and 11B show EC-SOD mRNA levels in the aorta (FIG. 11A) and left ventricle (FIG. 11B) of non-diabetic, diabetic and triethylenetetramine di hydrochloride treated rats.

FIGS. 12A and 12B show TGFβ-1 levels in the aorta (FIG. 12A) and left ventricle (FIG. 12B) of non-diabetic, diabetic and triethylenetetramine dihydrochloride treated rats.

FIGS. 13A and 13B show Collagen IV levels in the aorta (FIG. 13A) and left ventricle (FIG. 13B) of non-diabetic, diabetic and triethylenetetramine di hydrochloride treated rats.

FIGS. 14A and 14B show Smad4 levels in the aorta (FIG. 14A) and left ventricle (FIG. 14B) of non-diabetic, diabetic and triethylenetetramine dihydrochloride treated rats.

FIG. 15 shows a gel illustrating the effects of 5 mM spermine, spermidine and triethylenetetramine di hydrochloride on cytochrome C release.

FIG. 16 shows a gel illustrating the combination of 5 mM spermine with either triethylenetetramine dihydrochloride or spermidine, at concentrations of either 2.5 mM or 5 mM on cytochrome C release.

FIG. 17 shows the residual citrate synthase after spermine treatment of mitochondria.

FIG. 18 shows the residual citrate synthase after incubation with 5 mM of spermine, spermidine and triethylenetetramine dihydrochloride.

FIG. 19 shows the effect of triethylenetetramine dihydrochloride in combination with 5 mM spermine on the residual citrate synthase.

FIG. 20 shows the effect of spermidine in combination with 5 mM spermine on the residual citrate synthase.

FIG. 21 shows the respiration rates of different substrates on the different complexes of the electron transport chain of mitochondria isolated from left ventricle muscle of control, control treated with triethylenetetramine disuccinate, diabetic control and diabetic treated with triethylenetetramine disuccinate.

FIG. 22 is similar to FIG. 21, except shows the respiration rates on mitochondria isolated from permeabilised left ventricle endomyocardial fibres of the Spontaneous Hypertensive Rat (SHR) and the corresponding control rat model (WKY).

### DETAILED DESCRIPTION OF THE INVENTION

It has been discovered that certain compounds, including those described or referenced herein, can assist in normalizing lowered Cu⁺¹ levels. Example 1 examined the level of various elements in the left ventricle of three groups of rats: (1) normal (non-diabetic), (2) diabetic, and (3) diabetic treated with triethylenetetramine dihydrochloride. This Example shows that total copper, predominantly copper (I), is significantly decreased in the hearts of this animal model. Treatment with a copper (II) antagonist, in this case, triethylenetetramine dihydrochloride significantly increased total copper levels, normalizing copper levels to that of non-diabetic animals. There were also small but non-statistically significant decreases in zinc levels in the diabetic animals. Diabetic animals treated with triethylenetetramine dihydrochloride showed a significant increase in total zinc levels. Sodium, magnesium calcium, silicon, phosphorous, sulfur, chloride and potassium levels were not significantly changed between the three groups of animals.

It has been discovered that certain compounds, including those described or referenced herein, can mitigate mitochondrial swelling, elevated mitochondrial protein expression, and elevated expression of nuclear mitochondrial genes. Example 2 examined protein levels in the left ventricle of three groups of rats: (1) normal (non-diabetic), (2) diabetic, and (3) diabetic treated with triethylenetetramine di hydrochloride. Results showed that over 211 proteins were significantly changed in diabetic animals compared to non-diabetic animals. 33 of these proteins were significantly normalized by treatment with a copper (II) antagonist, in this case, triethylenetetramine dihydrochloride. Proteins that have been successfully identified include: NADH dehydrogenase (ubiquinone) 1 alpha subcomplex 10, subunit A of the succinate dehydrogenase complex, core protein I of the cytochrome bcl complex, α subunit of ATP synthase, and β subunit of ATP synthase, dihydrolipoamide S-acetyltransferase, di hydrolipoamide dehydrogenase, di hydroliposyllysine-residue succinyltransferase, carnitine O-palmitoyltransferase II, chain F of the enoyl-CoA hydratase, 3-hydroxyacyl-CoA dehydrogenase type II, Heat Shock Protein 60, B chain of L-lactate dehydrogenase, cytosolic malate dehydrogenase, annexin A3, and annexin A5. These proteins are found in the mitochondrial inner membrane, mitochondrial matrix, cytoplasm, plasma membrane, phagosomes, early endosomes, late endocytic organelles and mitochondria. Example 3 examined the effects of spermine, spermidine and triethylenetetramine dihydrochloride on distressed mitochondria isolated from non-diabetic and diabetic rats. Mitochondrial distress was induced by the administration of calcium and evidenced by mitochondrial swelling. Spermine, spermidine and triethylenetetramine dihydrochloride all inhibit mitochondrial swelling at concentrations below 0.625 mM. However at concentrations above 0.625 mM, spermine induced swelling, while spermidine and triethylenetetramine dihydrochloride continued to have a protective effect on the distressed mitochondria at all concentrations. The protective effect of triethylenetetramine dihydrochloride in an experiment carried out in the absence of calcium was striking. It was discovered that incubation of mitochondria with spermine led to swelling of mitochondria and the effect was concentration dependent up to 5 mM of spermine (the highest concentration tested). Simultaneous incubation with 5 mM spermine and increasing concentrations of triethylenetetramine di hydrochloride (up to 5 mM) protected against mitochondrial swelling. The effect of triethylenetetramine dihydrochloride on spermine induced swelling varied with concentration. Example 4 relates to mRNA expression in the left ventricle of non-diabetic and diabetic rats. Over 900 genes showed significant changes in expression between the diabetic and non-diabetic rats. mRNA expression for 16 proteins identified in Example 2 are specifically described. Carnitine O-palmitoyltransferase II had a 1.4 fold increase in expression in diabetic animals. Chain F of the enoyl-CoA hydratase was increased by 1.7-fold in the peroxisomal isoform in diabetic animals. 3-hydroxyacyl-CoA dehydrogenase type II was increased by 1.8 fold in diabetic animals, and annexin A7 was increased by 1.3 fold in diabetic animals. Furthermore, it has been discovered that certain compounds, including those described or referenced herein, can assist in lowering elevated TGF-β1, collagen IV and Smad 4 levels and increasing EC-SOD levels. Example 5 describes EC-SOD, TGF-β1, collagen IV, and Smad 4 RNA levels in the aorta and left ventricle of non-diabetic, diabetic and triethylenetetramine dihydrochloride treated diabetic rats. Results show that EC-SOD RNA expression was decreased in the aorta and left ventricle in diabetic animals. RNA levels were normalized by treatment of animals with a copper (II) antagonist, in this case triethylenetetramine dihydrochloride. TGF-β1, collagen IV, and Smad 4 RNA expression levels are significantly up-regulated in this animal model. This up-regulation was normalized with a copper (II) antagonist, in this case triethylenetetramine dihydrochloride. Example 6 examined the effects of spermidine and triethylenetetramine dihydrochloride on cytochrome c release and citrate sythase activity in spermine treated mitochondria isolated from lean (non-diabetic) ZDF rats. Levels of cytochrome c release were decreased in a dose dependent manner when treated with triethylenetetramine dihydrochloride. Cytochrome c release was also reduced, though to a lesser degree, by spermidine. Additionally, triethylenetetramine dihydrochloride normalized citrate synthase activity in spermine treated mitochondria. Spermidine also improved citrate synthase activity, although not as effectively as triethylenetetramine di hydrochloride. Example 7 examined the effects of triethylenetetramine disuccinate treatment on the mitochondria of diabetic and non-diabetic animals as compared to their untreated litter mates. Specifically, the respiration rates of complexes I to V of the electron transport chain (ETC) were analysed. Respiration flux through all complexes was depressed by approximately 40% in diabetic mitochondria relative to control mitochondria. Example 8 examined the effects of triethylenetetramine disuccinate treatment on the mitochondria of hypertensive rats (SHR) and non-hypertensive rats (WKY) as compared to their untreated control litter mates. Respiration flux through all complexes of the ETC were analysed where, except for GM2, all complexes of the ETC were significantly increased as compared to the untreated WKY model. In both examples 7 and 8, GM2—is the respiration flux through complex I in the absence of ADP and uncoupling agents (FCCP, dinitrophenol), which provides an indirect measure of the proton leak rate through the inner mitochondrial membrane (state 2 respiration). Flux rates determined following the addition of glutamate and malate and ADP (GM3) provides a measure of flux through complex I with phosphorylation (i.e. the phosphorylation of ADP to ATP, state-3 respiration). GMS3 provides a measure of state-3 flux through complexes I and II following respiration on glutamate (and an estimate of maximal flux in vivo). S3 provides an estimate of respiration using succinate as substrate (complex II) alone, following inhibition of complex I with rotenone. S4^{o} provides a measure of respiratory flux with complex V blocked by oligomycin (non-phosphorylating, similar to GM2). S4^{o} provides another measure of proton leak rate (4 refers to state 4 respiration where the superscript ^{o} refers to oligomycin, which artificially induces state 4 by blocking the ATPase complex V). COX provides a measure of respiration through complex IV (or cytochrome oxidase, COX), using TMPD and ascorbate as electron donors. COXc is the respiration flux rate in the presence of TMPD, ascorbate and saturating cytochrome c. The ratio of COXc/COX provides a measure of membrane stability as cytochrome c can be lost from the inner mitochondrial membrane due to damage to the outer mitochondrial membrane additional cytochrome c results in increased flux.

Reduced levels of copper in the mitochondria results in the reduction of cytochrome c oxidase activity, leading to increased electron leaking and increased oxidative stress. This cycle and its deleterious effects can be treated by administration of antagonists compounds, including preferred Cu⁺² antagonist agents including Cu⁺² chelating agents.

It has also been discovered that certain compounds, including those described or referenced herein, can lessen elevated mitochondria number.

The present inventions relate generally to compounds, compositions and methods for treating mitochondria-associated diseases, including respiratory chain disorders. The inventions also relate to diseases and disorders in which free radical mediated oxidative injury leads to tissue degeneration, and diseases and disorders in which cells inappropriately undergo programmed cell death (apoptosis), leading to tissue degeneration.

The present inventions also relate to compositions and methods for treating such disease and disorders through the use of compounds which function as, respectively, mitochondria protecting agents, mitochondria biogenesis agents, and anti-apoptotic agents.

The present inventions are directed in part to the treatment of mitochondria-associated diseases by administration to a mammal in need thereof an effective amount of a copper binding polyamine compound, polyamine compounds that bind Cu⁺², and preferably polyamine compounds that are specific for Cu⁺² over Cu⁺¹. Polyamine compounds may include, for example, spermine, as well as spermidine and other tetramines. Preferred tetramine compounds include triethylenetetramine (2,2,2 tetramine), 2,3,2 tetramine and 3,3,3 tetramine as well as salts, active metabolites, derivatives, and prodrugs thereof. Other pharmaceutically acceptable polyamines are also contemplated.

The present inventions are also directed in part to the treatment of mitochondria-associated diseases by administration to a mammal in need thereof an effective amount of a compound according to Formula (I) or Formula (II).

In still further embodiments, methods are provided for treating mitochondria-associated diseases by administering one or more copper binding tetramine compounds, compounds of Formula (I), or compounds of Formula (II), in the form of a pharmaceutical composition. Thus, pharmaceutical compositions are also provided comprising one or more copper binding tetramine compounds, compounds of Formula (I), or compounds of Formula (II), in combination with a pharmaceutically acceptable carrier or diluent.

Copper antagonists useful in the invention also include copper chelators that have been pre-complexed with a non-copper metal ion prior to administration for therapy. Metal ions used for pre-complexing have a lower association constant for the copper antagonist than that of copper. For example, a metal ion for pre-complexing a copper antagonist that chelates Cu²⁺ is one that has a lower binding affinity for the copper antagonist than Cu²⁺. Preferred metal ions for precomplexing include calcium (e.g., Ca²⁺), magnesium (e.g., Mg²⁺), chromium (e.g., Cr²⁺ and Cr³⁺), manganese (e.g., Mn²⁺), zinc (e.g., Zn²⁺), selenium (e.g., Se⁴⁺), and iron (e.g., Fe²⁺ and Fe³⁺). Most preferred metal ions for precomplexing are calcium, zinc, and iron. Other metals include, for example, cobalt (e.g., Co²⁺), nickel (e.g., Ni²⁺), silver (e.g., Ag¹⁺), and bismuth (e.g., Bi³⁺). Metals are chosen with regard, for example, to their relative binding to the copper antagonist, and relative to toxicity and the dose of the copper antagonist to be administered.

Also encompassed are metal complexes comprising copper antagonists and non-copper metals (that have lower binding affinities than copper for the copper antagonist) and one or more additional ligands than typically found in complexes of that metal. These additional ligands may serve to block sites of entry into the complex for water, oxygen, hydroxide, or other species that may undesirably complex with the metal ion and can cause degradation of the copper antagonist. For example, copper complexes of triethylenetetramine have been found to form pentacoordinate complexes with a tetracoordinated triethylenetetramine and a chloride ligand when crystallized from a salt solution rather than a tetracoordinate Cu²⁺ triethylenetetramine complex. In this regard, 219 mg of triethylenetetramine .2 HCl were dissolved in 50 ml, and 170 mg of CuCl₂.2H2O were dissolved in 25 ml ethanol (95%). After addition of the CuCl₂ solution to the triethylenetetramine solution, the color changed from light to dark blue and white crystals precipitated. The crystals were dissolved by addition of a solution of 80 mg NaOH in 15 ml H2O. After the solvent was evaporated, the residue was dissolved in ethanol, and two equivalents of ammonium-hexafluorophosphate were added. Blue crystals could be obtained after reduction of the solvent. Crystals were found that were suitable for x-ray structure determination. X-ray crystallography revealed a [Cu(triethylenetetramine)CI] complex. Other coordinated complexes may be formed from or between copper antagonists, for example, copper chelators (such as Cu2+ chelators, spermidine, spermine, tetracyclam, etc.), particularly those subject to degradative pathways such as those noted above, by providing additional complexing agents (such as anions in solution, for example, I⁻, Br⁻, F⁻, (SO₄)²⁻, (CO₃)²⁻, BF⁴⁻, NO³⁻, ethylene, pyridine, etc.) in solutions of such complexes. This may be particularly desirable for complexes with more accessible metal ions, such as planar complexes or complexes having four or fewer coordinating agents, where one or more additional complexing agents could provide additional shielding to the metal from undesirable ligands that might otherwise access the metal and displace a desired complexing agent.

In the context of the inventions, mitochondria-associated diseases include diseases in which free radical mediated oxidative injury leads to tissue degeneration, and diseases in which cells inappropriately undergo apoptosis, and include the treatment of a wide number of mitochondria-associated diseases, including but not limited to auto-immune disease, Alpers Disease (progressive infantile poliodystrophy, Barth syndrome, congenital muscular dystrophy, fatal infantile myopathy, “later-onset” myopathy, MELAS (mitochondrial encephalopathy, lactic acidosis, and stroke), MIDD (mitochondrial diabetes and deafness), MERRF (myoclonic epilepsy ragged red fiber syndrome), arthritis, NARP (Neuropathy; Ataxia; Retinitis Pigmentosa), MNGIE (Myopathy and external ophthalmoplegia; Neuropathy; Gastro-Intestinal; Encephalopathy), LHON (Leber's; Hereditary; Optic; Neuropathy), Kearns-Sayre disease, Pearson's Syndrome, PEO (Progressive External Ophthalmoplegia), Wolfram syndrome, DIDMOAD (Diabetes Insipidus, Diabetes Mellitus, Optic Atrophy, Deafness), ADPD (Alzheimer's disease; Parkinson's disease), AMFD (ataxia, myoclonus and deafness), CIPO (chronic intestinal pseudoobstruction; myopathy; opthalmoplegia), CPEO (chronic progressive external opthalmoplegia), maternally inherited deafness, aminoglycoside-induced deafness, DEMCHO (dementia; chorea), DMDF (diabetes mellitus; deafness), exercise intolerance, ESOC (epilepsy; strokes; optic atrophy; congenitive decline), FBSN (familial bilateral striatal necrosis), FICP (fatal infantile cardiomyopathy plus a MELAS-associated cardiomyopathy), GER (gastrointestinal reflux), LCHAD (Long-Chain Hydroxyacyl-CoA Dehydrogenase Deficiency), SCHAD (Sharot-Chain Hydroxyacyl-CoA Dehydrogenase Deficiency), MAD (Multiple Acyl-CoA Dehydrogenase Deficiency) MCAD (Medium-Chain Acyl-CoA Dehydrogenase Deficiency), SCAD (Short-Chain Acyl-CoA Dehydrogenase Deficiency), VLCAD (very long-chain Acyl-CoA Dehydrogenase Deficiency), LIMM (lethal infantile mitochondrial myopathy), LDYT (Leber's hereditary optic neuropathy and DYsTonia), Luft Disease, MDM (myopathy; diabetes mellitus), MEPR (myoclonic epilepsy; psychomotor regression), MERME (MERRF/MELAS overlap disease), MHCM (maternally inherited hypertrophic cardiomyopathy), MICM (maternally inherited cardiomyopathy), MILS (maternally inherited Leigh syndrome), mitochondrial encephalocardiomyopathy, mitochondrial encephalomyopathy, mitochondrial myopathy, MMC (maternal myopathy; cardio myopathy), multisystem mitochondrial disorder (myopathy; encephalopathy; blindness; hearing loss; peripheral neuropathy), NIDDM (non-insulin dependent diabetes mellitus), Pearson Syndrome PEM (progressive encephalopathy), PME (progressive myclonus epilepsy), Rett syndrome, SIDS (sudden infant death syndrome, SNHL (sensorineural hearing loss), Leigh's Syndrome, dystonia, schizophrenia, and psoriasis.

As used herein, a “copper antagonist” is a pharmaceutically acceptable compound that binds or chelates copper, preferably copper (II), in vivo for removal. Copper chelators are presently preferred copper antagonists. Copper (II) chelators, and copper (II) specific chelators (i.e., those that preferentially bind copper (II) over other forms of copper such as copper (I)), are especially preferred. “Copper (I)” refers to the +1 form of copper, also sometimes referred to as Cu⁺¹. “Copper (II)” refers to the oxidized (or +2) form of copper, also sometimes referred to as Cu⁺².

As used herein, a “disorder” is any disorder, disease, or condition that would benefit from an agent as disclosed herein. Particularly preferred are agents that reduce extracellular copper or extracellular copper concentrations (local or systemic) and, more particularly, agents that reduce extracellular copper (II) or extracellular copper (II) concentrations (local or systemic). Disorders include, but are not limited to, those described and/or referenced herein, and include diseases, disorders and conditions include that would benefit from a decrease in mitochondrial number, a decrease in mitochondrial protein expression, a decrease in expression of nuclear mitochondrial genes, a decrease in mitochondrial swelling, a decrease in TGFβ-1 levels, a decrease in Smad 4 levels, a decrease in collagen IV levels and/or an increase in Cu⁺¹ levels.

As used herein, “mammal” refers to any animal classified as a mammal, including humans, domestic and farm animals, and zoo, sports, or pet animals, such as dogs, horses, cats, sheep, pigs, cows, etc. The preferred mammal herein is a human.

As used herein, “pharmaceutically acceptable salts” refers to salts prepared from pharmaceutically acceptable non-toxic bases or acids including inorganic or organic bases and inorganic or organic acids the like. When a compound is basic, for example, salts may be prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are hydrochloric and succinic acid copper antagonist salts. Succinic acid copper antagonist salts are most preferred, particularly for those copper antagonist salts that are not anhydrous.

As used herein, “preventing” means preventing in whole or in part, or ameliorating or controlling.

As used herein, a “therapeutically effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In one aspect of the present inventions, the result will involve the prevention, decrease, or reversal of mitochondrial injury, in whole or in part, and prevention and/or treatment of related diseases, disorders and conditions, including those referenced herein. Therapeutic effects include, for example, a decrease in mitochondrial number, a decrease in mitochondrial protein expression, a decrease in expression of nuclear mitochondrial genes, a decrease in mitochondrial swelling, a decrease in TGFβ-1 levels, a decrease in Smad 4 levels, a decrease in collagen IV levels and/or an increase in Cu⁺¹ levels.

As used herein, the term “treating” refers to both therapeutic treatment and prophylactic or preventative measures. Those in need of treatment include those already with the disorder as well as those prone to having the disorder, or those diagnosed with the disorder, or those in which the disorder is to be prevented.

The present invention also provides methods to increase copper (I) by decreasing copper (II).

The invention is also provides a method of increasing copper (I) levels by administering a pharmaceutically effective amount of a copper (II) antagonist. Furthermore, the invention is directed to the treatment or prevention of copper related disease disorders and conditions associated with, or characterized at least in part by reduced copper (I) levels, including, but not limited to anemia, baldness, heart palpitation, hypothyroid disease, cerebral aneurysm, stroke, osteoporosis, bone fractures, periodontal disease, nervous system disorders, including ataxia, rheumatoid arthritis, ulcerative collitus, Crohn's disease, Menke's Syndrome, reduced HDL cholesterol, increased HDL cholesterol, decreased leukocytes, hypopigmentation in the hair and skin, weakness, fatigue, skin sores and breathing difficulties.

Reduction in extracellular copper, generally in the copper II form, will be advantageous in the treatment of disorders, diseases, and/or conditions, caused or exacerbated by mechanisms that may be affected by a decrease in mitochondrial number, a decrease in mitochondrial protein expression, a decrease in expression of nuclear mitochondrial genes, a decrease in mitochondrial swelling, a decrease in TGFβ-1 levels, a decrease in Smad 4 levels, a decrease in collagen IV levels and/or an increase in Cu⁺¹ levels.

Nitrogen-containing copper antagonists, for example, such as, for example, triethylenetetramine, that can be delivered as a salt(s) (such as acid addition salts, e.g., triethylenetetramine disuccinate or triethylenetetramine dihydrochloride) act as copper-chelating agents or antagonists, which aids the elimination of copper from the body by forming a stable soluble complex that is readily excreted by the kidney. Thus inorganic acids can be used, e.g., sulfuric acid, nitric acid, hydrohalic acids such as hydrochloric acid or hydrobromic acid, phosphoric acids such as orthophosphoric acid, sulfamic acid. This is not an exhaustive list. Other organic acids can be used to prepare suitable salt forms, in particular aliphatic, alicyclic, araliphatic, aromatic or heterocyclic mono- or polybasic carboxylic, sulfonic or sulfuric acids, (e.g., formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methanesulfonic acid, ethanesulfonic acid, ethanedisulfonic acid, 2-hydroxyethanesulfonic acid, benzenesulfonic acid, p-toluenesulfonic acid, naphthalenemono-and-disulfonic acids, and laurylsulfuric acid). Hydrochloric acid and succinic acid salts are preferred, and succinic acid salts are most preferred. Those in the art will be able to prepare other suitable salt forms.

Nitrogen-containing copper antagonists, for example, such as, for example, triethylenetetramine, can also be in the form of quarternary ammonium salts in which the nitrogen atom carries a suitable organic group such as an alkyl, alkenyl, alkynyl or aralkyl moiety. In one embodiment such nitrogen-containing copper antagonists are in the form of a compound or buffered in solution and/or suspension to a near neutral pH much lower than the pH 14 of a solution of triethylenetetramine itself.

Other copper antagonists include derivatives, for example, triethylenetetramine in combination with picolinic acid (2-pyridinecarboxylic acid). These derivatives include, for example, triethylenetetramine picolinate and salts of triethylenetetramine picolinate, for example, triethylenetetramine picolinate HCl. They also include, for example, triethylenetetramine di-picolinate and salts of triethylenetetramine di-picolinate, for example, triethylenetetramine di-picolinate HCl. Picolinic acid moieties may be attached to triethylenetetramine, for example one or more of the CH₂ moieties, using chemical techniques known in the art. Those in the art will be able to prepare other suitable derivatives, for example, triethylenetetramine-PEG derivatives, which may be useful for particular dosage forms including oral dosage forms having increased bioavailability.

Other compounds include cyclic and acyclic compounds according to the following formulae, for example:

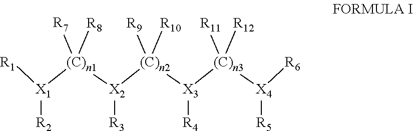

Tetra-heteroatom acyclic compounds within Formula I are provided where X₁, X₂, X₃, and X₄ are independently chosen from the atoms N, S or O, such that,
(a) for a four-nitrogen series, i.e., when X₁, X₂, X₃, and X₄ are N then: R₁, R₂, R₃, R₄, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₄, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(b) for a first three-nitrogen series, i.e., when X₁, X₂, X₃, are N and X₄ is S or O then: R₆ does not exist; R₁, R₂, R₃, R₄ and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(c) for a second three-nitrogen series, i.e., when X₁, X₂, and X₄ are N and X₃ is O or S then: R₄ does not exist and R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(d) for a first two-nitrogen series, i.e., when X₂ and X₃ are N and X₁ and X₄ are O or S then: R₁ and R₆ do not exist; R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(e) for a second two-nitrogen series, i.e., when X₁ and X₃ are N and X₂ and X₄ are O or S then: R₃ and R₆ do not exist; R₁, R₂, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(f) for a third two-nitrogen series, i.e., when X₁, and X₂ are N and X₃ and X₄ are O or S then: R₄ and R₆ do not exist; R₁, R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(g) for a fourth two-nitrogen series, i.e., when X₁ and X₄ are N and X₂ and X₃ are O or S then: R₃ and R₄ do not exist; R₁, R₂, R₅ and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Second, for a tetra-heteroatom series of cyclic analogues, one of R₁ and R₂ and one of R₅ and R₆ are joined together to form the bridging group (CR₁₃R₁₄)n4, and X₁, X₂, X₃, and X₄ are independently chosen from the atoms N, S or O such that,
(a) for a four-nitrogen series, i.e., when X₁, X₂, X₃, and X₄ are N then: R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(b) for a three-nitrogen series, i.e., when X₁, X₂, X₃, are N and X₄ is S or O then: R₅ does not exist; R₂, R₃, and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃ or R₄ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(c) for a first two-nitrogen series, i.e., when X₂ and X₃ are N and X₁ and X₄ are O or S then: R₂ and R₅ do not exist; R₃ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R3, or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(d) for a second two-nitrogen series, i.e., when X₁ and X₃ are N and X₂ and X₄ are O or S then: R₃ and R₅ do not exist; R₂ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R₂, or R₄ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(e) for a one-nitrogen series, i.e., when X₁ is N and X₂, X₃ and X₄ are O or S then: R₃, R₄ and R₅ do not exist; R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

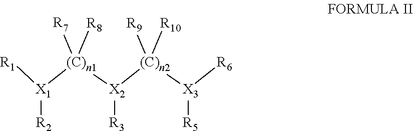

Tri-heteroatom compounds within Formula II are provided where X₁, X₂, and X₃ are independently chosen from the atoms N, S or O such that,
(a) for a three-nitrogen series, when X₁, X₂, and X₃ are N then: R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₅ or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, or R₁₀ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(b) for a first two-nitrogen series, when X₁ and X₂ are N and X₃ is S or O then: R₃ does not exist; R₁, R₂, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₅ or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, or R₁₀ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(c) for a second, two-nitrogen series, when X₁ and X₂ are N and X₃ is O or S then: R₅ does not exist; R₁, R₂, R₃, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1 and n2 are independently chosen to be 2 or 3; and R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, or R₁₀ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

A series of tri-heteroatom cyclic analogues according to the above Formula II are provided in which R₁ and R₆ are joined together to form the bridging group (CR₁₁R₁₂)n3, and X₁, X₂ and X₃ are independently chosen from the atoms N, S or O such that:
(a) for a three-nitrogen series, when X₁, X₂, and X₃ are N then: R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(b) for a two-nitrogen series, when X₁ and X₂ are N and X₃ is S or O then: R₅ does not exist; R₂, and R₃ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R₂ or R₃ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.
(c) for a one-nitrogen series, when X₁ is N and X₂ and X₃ are O or S then:
   i. R₃ and R₅ do not exist; R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

The compounds of the invention, including triethylenetetramine active agents, may be made using any of a variety of chemical synthesis, isolation, and purification methods known in the art. Exemplary synthetic routes are described below.

General synthetic chemistry protocols are somewhat different for these classes of molecules due to their propensity to chelate with metallic cations, including copper. Glassware should be cleaned and silanized prior to use. Plasticware should be chosen specifically to have minimal presence of metal ions. Metal implements such as spatulas should be excluded from any chemistry protocol involving chelators. Water used should be purified by sequential carbon filtering, ion exchange and reverse osmosis to the highest level of purity possible, not by distillation. All organic solvents used should be rigorously purified to exclude any possible traces of metal ion contamination.

Care must also be take with purification of such derivatives due to their propensity to chelate with a variety of cations, including copper, which may be present in trace amounts in water, on the surface of glass or plastic vessels. Once again, glassware should be cleaned and silanized prior to use. Plasticware should be chosen specifically to have minimal presence of metal ions. Metal implements such as spatulas should be avoided, and water used should be purified by sequential carbon filtering, ion exchange and reverse osmosis to the highest level of purity possible, and not by distillation. All organic solvents used should be rigorously purified to exclude any possible traces of metal ion contamination. Ion exchange chromatography followed by lyophilization is typically the best way to obtain pure solid materials of these classes of molecules. Ion exchange resins should be washed clean of any possible metal contamination.

Many of the synthetic routes allow for control of the particular R groups introduced. For synthetic methods incorporating amino acids, synthetic amino acids can be used to incorporate a variety of substituent R groups. The dichloroethane synthetic schemes also allow for the incorporation of a wide variety of R groups by using dichlorinated ethane derivatives. It will be appreciated that many of these synthetic schemes can lead to isomeric forms of the compounds; such isomers can be separated using techniques known in the art.

Documents describing aspects of these synthetic schemes include the following: (1) A W von Hoffman, Berichte 23, 3711 (1890); (2) The Polymerization Of Ethylenimine, Giffin D. Jones, Arne Langsjoen, Sister Mary Marguerite Christine Neumann, Jack Zomlefer, J. Org. Chem., 1944; 9(2); 125-147; (3) The peptide way to macrocyclic bifunctional chelating agents: synthesis of 2-(p-nitrobenzyl)-1,4,7,10-tetraazacyclododecane-N,N′,N″,N′″-tetraacetic acid and study of its yttrium(III) complex, Min K. Moi et al., J. Am. Chem. Soc., 1988; 110(18); 6266-6267; (4) Synthesis of a kinetically stable ⁹⁰Y labelled macrocycle-antibody conjugate, Jonathan P L Cox, et al., J. Chem. Soc. Chem. Comm., 797 (1989); (5) Specific and stable labeling of antibodies with technetium-99m with a diamide dithiolate chelating agent, Fritzberg A R, Abrams P G, Beaumier P L, Kasina S, Morgan A C, Rao T N, Reno J M, Sanderson J A, Srinivasan A, Wilbur D S, et al., Proc. Natl. Acad. Sc.i U.S.A. 85(11):4025-4029 (1988 June); (6) Towards tumour imaging with ¹¹¹In labelled macrocycle-antibody conjugates, Andrew S Craig et al., J. Chem. Soc. Chem. Comm., 794 (1989); (7) Synthesis of C- and N-functionalised derivatives of NOTA, DOTA, and DTPA: bifunctional complexing agents for the derivitisation of antibodies, Jonathan P L Cox et al. J. Chem. Soc. Perkin. I, 2567 (1990); (8) Macrocyclic chelators as anticancer agents in radioimmunotherapy, N R A Beeley and P R J Ansell, Current Opinions in Therapeutic Patents, 2:1539-1553 (1992); and (9) Synthesis of new macrocyclic amino-phosphinic acid complexing agents and their C- and P-functionalised derivatives for protein linkage, Christopher J Broan et al., Synthesis, 63 (1992).

Acyclic and cyclic compounds of the invention and exemplary synthetic methods and existing syntheses from the art include the following:

### For Tetra-Heteroatom Acyclic Examples of Formula I

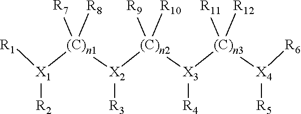

X₁, X₂, X₃, and X₄ are independently chosen from the atoms N, S or O such that:
4N series:
when X₁, X₂, X₃, and X₄ are N then:
R₁, R₂, R₃, R₄, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₄, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Also provided are embodiments wherein one, two, three or four of R₁ through R₁₂ are other than hydrogen.

In some embodiments, the compounds of Formula I or II are selective for a particular oxidation state of copper. For example, the compounds may be selected so that they preferentially bind oxidized copper, or copper (II). Copper selectivity can be assayed using methods known in the art. Competition assays can be done using isotopes of copper (I) and copper (II) to determine the ability of the compounds to selectively bind one form of copper.

In some embodiments, the compounds of Formula I or II may be chosen to avoid excessive lipophilicity, for example by avoiding large or numerous alkyl substituents. Excessive lipophilicity can cause the compounds to bind to and/or pass through cellular membranes, thereby decreasing the amount of compound available for chelating copper, particularly for extracellular copper, which may be predominantly in the oxidized form of copper (II).

### Synthesis of Examples of the Open Chain 4N Series of Formula I

Triethylenetetramine itself has been synthesized by reaction of 2 equivalents of ethylene diamine with 1,2-dichloro ethane to give triethylenetetramine directly (1). Modification of this procedure by using starting materials with appropriate Rₐ and R_{b} groups (where Rₐ, R_{b}=R₇, R₈ or R₁₁, R₁₂) would lead to symmetrically substituted open chain 4N examples as shown below:

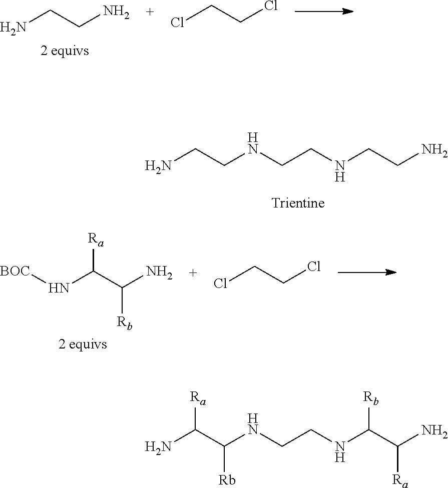

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tetra-aza series. In order to obtain the un-symmetrically substituted derivatives a variant of some chemistry described by Meares et al (3) should be used. Standard peptide synthesis using the Rink resin along with FMOC protected natural and un-natural amino acids which can be conveniently cleaved at the penultimate step of the synthesis generates a tri-peptide C-terminal amide. This is reduced using Diborane in THF to give the open chain tetra-aza compounds as shown below:

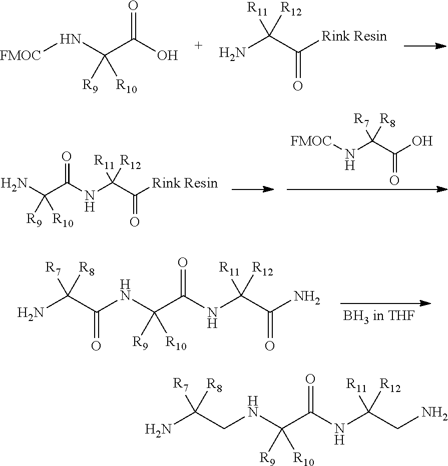

The incorporation of R₁, R2, R₅ and R₆ can be accomplished with this chemistry by standard procedures.

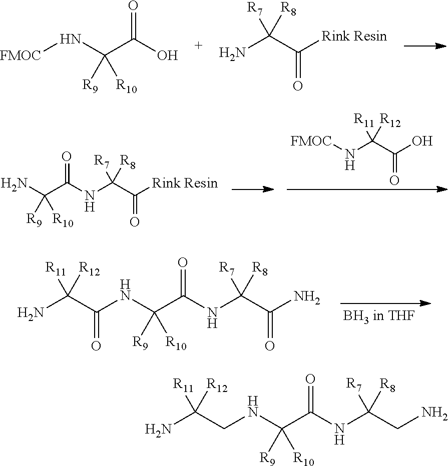

The reverse Rink approach, shown above, also leads to this class of tetra-aza derivatives and may be useful in cases where peptide coupling of a sterically hindered amino acid requires multiple coupling attempts in order to achieve success in the initial Rink approach.

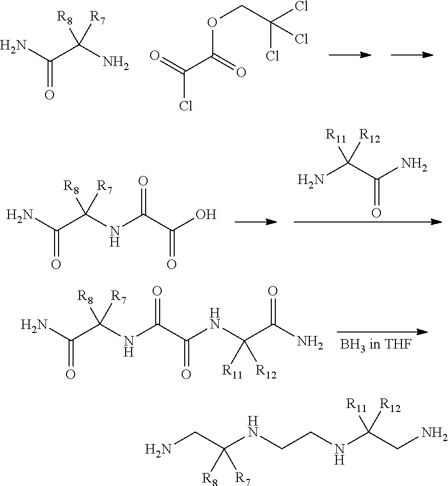

The oxalamide approach, shown above, also can lead to successful syntheses of this class of compounds, although the central substituents are always going to be hydrogen or its isotopes with this kind of chemistry. This particular variant makes use of the trichloroethyl ester group to protect one of the carbolxylic acid functions of oxalic acid but other protecting groups are also envisaged. Reaction of an amino acid amide derived from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative.
3NX series 1:
when X₁, X₂, X₃, are N and X₄ is S or O then:
R₆ does not exist
R₁, R₂, R₃, R₄ and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Open Chain 3NX Series 1 of Formula I

Variations of the syntheses used for the 4N series provide examples of the 3N series 1 class of compounds. The chemistry described by Meares et al (3) can be modified to give examples of the 3NX series of compounds.

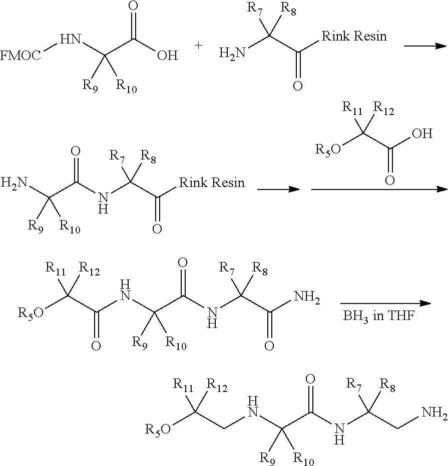

Standard peptide synthesis according to the so-called reverse Rink approach as shown above using FMOC protected natural and un-natural amino acids which can be conveniently cleaved at the penultimate step of the synthesis generates a modified tri-peptide C-terminal amide. The cases where X₄ is O are incorporated by the use of an alpha-substituted carboxylic acid in the last coupling step. This is reduced using Diborane in THF to give the open chain tetra-aza compounds.

The incorporation of R₁, R₂, R₅ and R₆ can be accomplished with this chemistry by standard procedures.

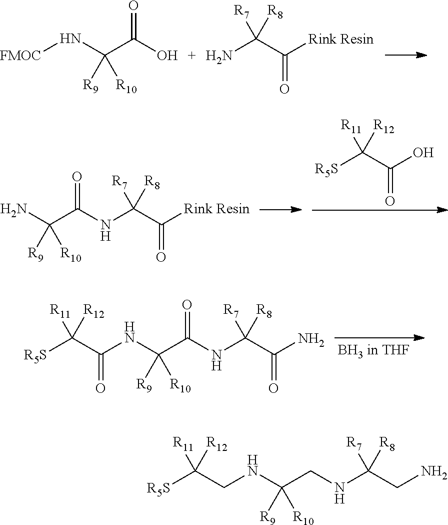

For the cases where X₄=S a similar approach using standard peptide synthesis according to the so-called reverse Rink approach as shown above can be used. Coupling with FMOC protected natural and un-natural amino acids, which can be conveniently cleaved at the penultimate step of the synthesis, generates a modified tri-peptide C-terminal amide. The incorporation of X₄=S is achieved by the use of an alpha-substituted carboxylic acid in the last coupling step. This is reduced using Diborane in THF to give the open chain tetra-aza compounds.

The incorporation of R₁, R₂, R₅ and R₆ can be accomplished with this chemistry by standard procedures.

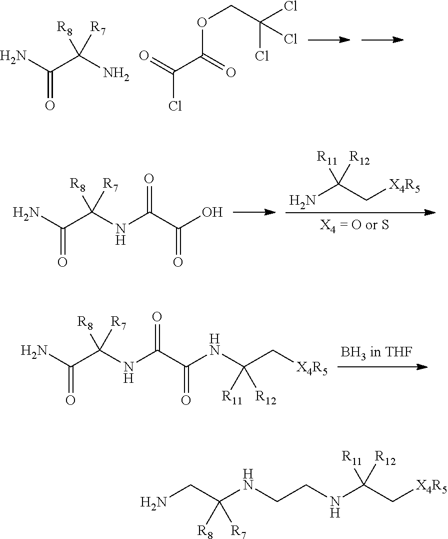

The oxalamide approach, shown above, can also lead to successful syntheses of this class of compounds, although the central substituents are always going to be hydrogen or its isotopes with this kind of chemistry. This particular variant makes use of the trichloroethyl ester group to protect one of the carbolxylic acid functions of oxalic acid but other protecting groups are also envisaged. Reaction of an amino acid amide derived from a natural or unnatural amino acid with a differentially protected oxalyl mono chloride gives the mono-oxalamide shown which can be reacted under standard peptide coupling conditions with an ethanolamine or ethanethiolamine derivative to give the un-symmetrical bis-oxalamide which can then be reduced with diborane as shown to give the desired tri-aza derivative.
3NX series 2:
when X₁, X₂, and X₄ are N and X₃ is O or S then:
R₄ does not exist, and
R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Open Chain 3NX Series 2 of Formula I

A different approach can be used for the synthesis of the 3N series 2 class of compounds. The key component is the incorporation in the synthesis of an appropriately substituted and protected ethanolamine or ethanethiolamine derivative, which is readily available from both natural and un-natural amino acids, as shown below.

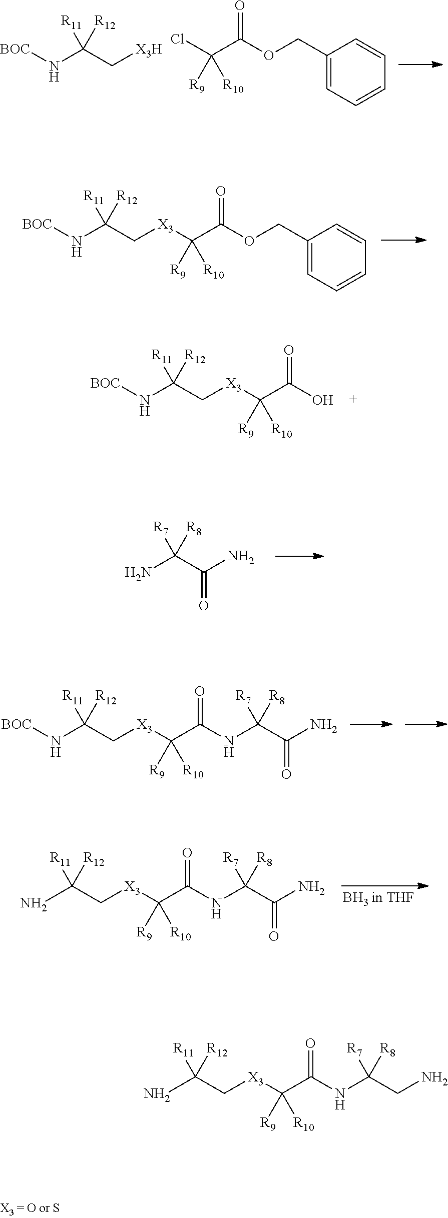

The BOC protected ethanolamine or ethanethiolamine is reacted with an appropriate benzyl protected alpha chloroacid. After hydrogenation to deprotect the ester function, standard peptide coupling with a natural or unnatural amino acid amide followed by deprotection and reduction with diborane in THF gives the open chain tri-aza compounds. If hydrogenation is not compatible with other functionality in the molecule then alternative combinations of protecting groups can be used such as trichloroethyloxy carbonyl and t-butyl.

The incorporation of R₁, R₂, R₅ and R₆ can be accomplished with this chemistry by standard procedures.
2N2X series 1:
when X₂ and X₃ are N and X₁ and X₄ are O or S then:
R₁ and R₆ do not exist;
R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or several of R₂, R₃, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Open Chain 2N2X Series 1 of Formula I

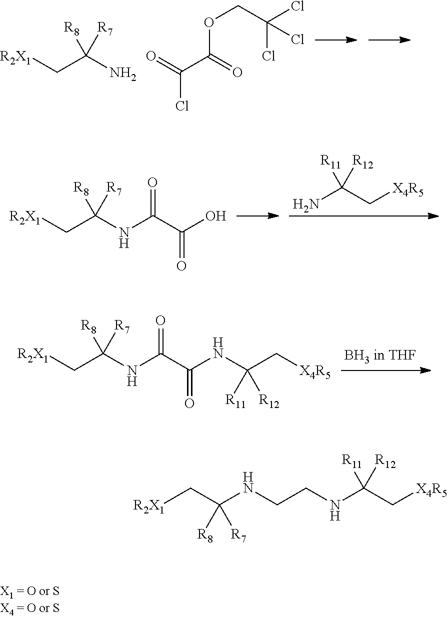

The oxalamide approach, shown above, can lead to successful syntheses of this class of compounds. This particular variant makes use of the trichloroethyl ester group to protect one of the carbolxylic acid functions of oxalic acid but other protecting groups are also envisaged. Reaction of an aminoalcohol or aminothiol derivative readily available from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative.

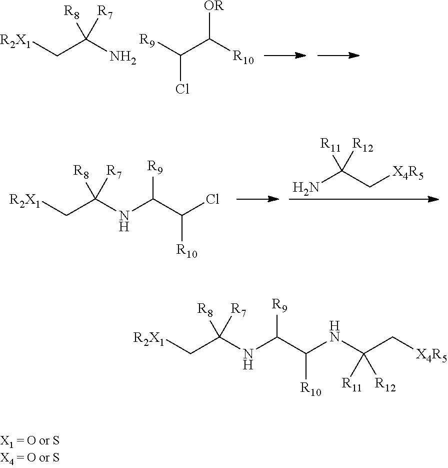

A variant of the dichloroethanee approach, shown above, can also lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriate aminoalcohol or aminothiol derivative readily available from a natural or unnatural amino acid to give the un-symmetrical desired product.
2N2X series 2:
when X₁ and X₃ are N and X₂ and X₄ are O or S then:
R₃ and R₆ do not exist;
R₁, R₂, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of the Open Chain 2N2X Series 2 of Formula I

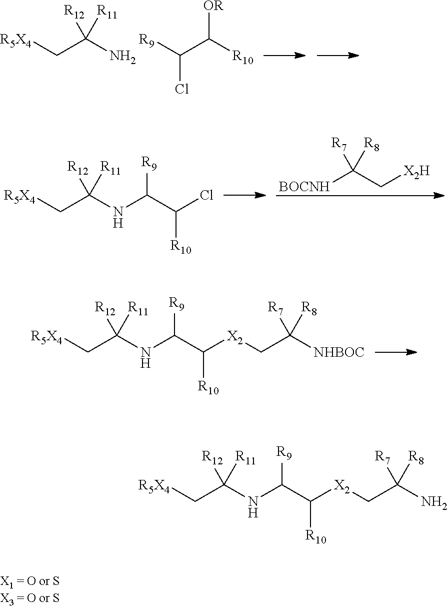

A variant of the dichloroethane approach, shown above, can lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriately protected aminoalcohol or aminothiol derivative, readily available from a natural or unnatural amino acid, to give the un-symmetrical desired product after de-protection.
2N2X series 3:
when X₁ and X₂ are N and X₃ and X₄ are O or S then:
R₄ and R₆ do not exist;
R₁, R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2N2X Series 3

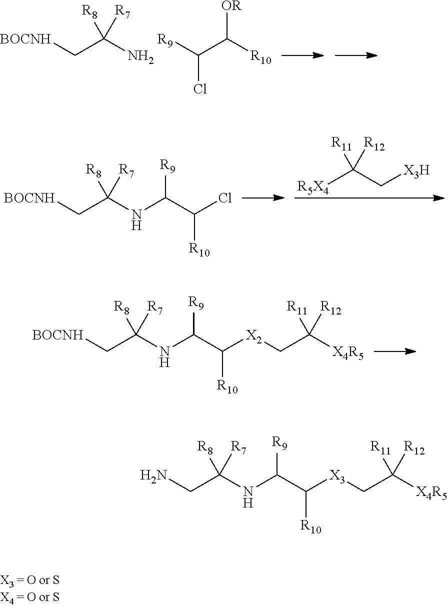

A variant of the dichloroethanee approach, shown above, can lead to successful syntheses of this class of compounds. Reaction of a monoprotected ethylene diamine derivative, readily available from a natural or unnatural amino acid with an O-protected 1-chloro, 2-hydroxy ethane derivative followed by deprotection and substitution with chloride gives the mono-chloro compound shown which can be further reacted with an appropriately protected bis-alcohol or bis thiol derivative, readily available from a natural or unnatural amino acid, to give the un-symmetrical desired product after de-protection.
2N2X series 4:
when X₁ and X₄ are N and X₂ and X₃ are O or S then:
R₃ and R₄ do not exist;
R₁, R₂, R₅ and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2, and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2N2X Series 4 of Formula I

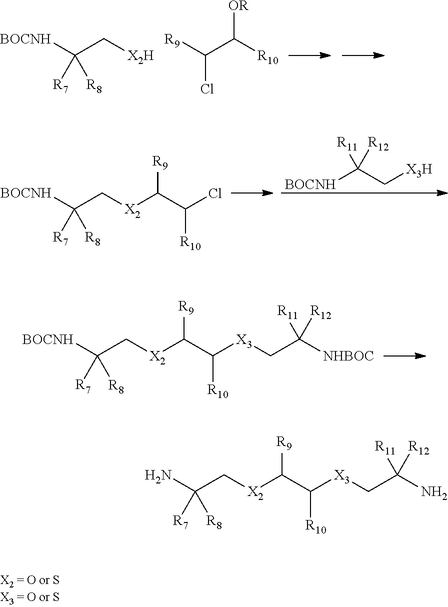

A variant of the dichloroethanee approach, shown above, can lead to successful syntheses of this class of compounds. Reaction of a an appropriately protected bis-alcohol or bis thiol derivative, readily available from a natural or unnatural amino acid, with an O-protected 1-chloro, 2-hydroxy ethane derivative followed by deprotection and substitution with chloride gives the mono-chloro compound shown which can be further reacted with an appropriately protected bis-alcohol or bis thiol derivative, readily available from a natural or unnatural amino acid, to give the un-symmetrical desired product after de-protection.

For the Tetra-Heteroatom Cyclic Series:

One of R₁ and R₂ (if R₁ does not exist) and one of R₅ (if R₆ does not exist) and R₆ are joined together to form the bridging group (CR₁₃R₁₄)n4;

X₁, X₂, X₃, and X₄ are independently chosen from the atoms N, S or O such that: 4N macrocyclic series:

when X₁, X₂, X₃, and X₄ are N then:

R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, n3, and n4 are independently chosen to be 2 or 3, and each repeat of any of n1, n2, n3 and n4 may be the same as or different than any other repeat; and

R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 4N Series of Formula I

Triethylenetetramine itself has been synthesized by reaction of 2 equivalents of ethylene diamine with 1,2-dichloro ethane to give triethylenetetramine directly (1). Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate Rₐ and R_{b} (where Rₐ, R_{b} correspond to R₇, R₈ or R₁₁, R₁₂) groups would lead to symmetrically substituted 12N4 macrocycle examples as shown below:

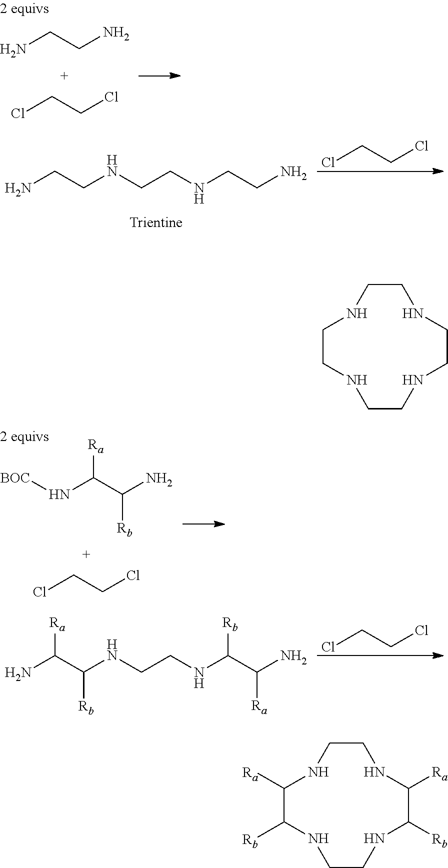

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tetra-aza series. In order to obtain the un-symmetrically substituted derivatives a variant of some chemistry described by Meares et al (3) should be used. Standard peptide synthesis using the Merrifield approach or the SASRIN resin along with FMOC protected natural and un-natural amino acids which can be conveniently cleaved at a later step of the synthesis generates a fully protected tetra-peptide C-terminal SASRIN derivative. Cleavage of the N terminal FMOC protecting group followed by direct cyclization upon concomitant cleavage from the resin gives the macrocyclic tetrapeptide. This is reduced using Diborane in THF to give the 12N4 series of compounds as shown below:

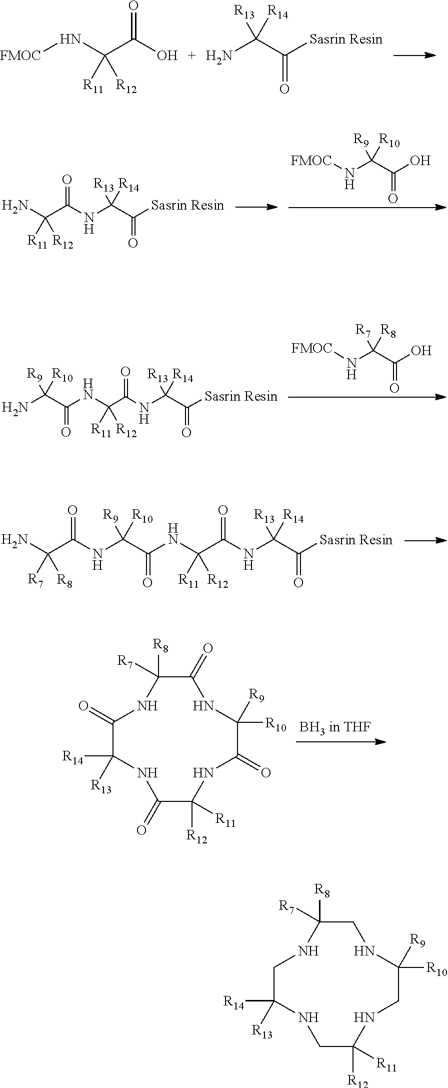

The incorporation of R₁, R₂, R₅ and R₆ can be accomplished with this chemistry by standard procedures.

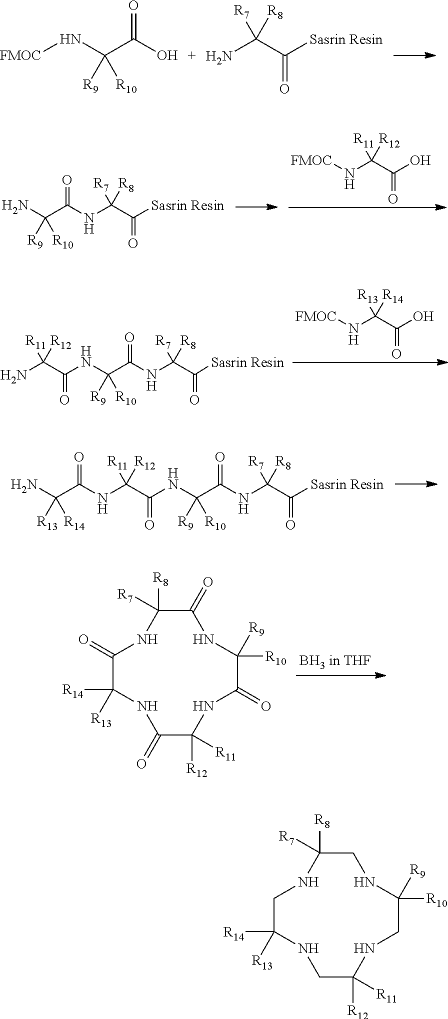

The reverse Merrifield/SASRIN approach, shown above, also leads to this class of tetra-aza derivatives and may be useful in cases where peptide coupling of a sterically hindered amino acid requires multiple coupling attempts in order to achieve success in the initial Merrifield approach.

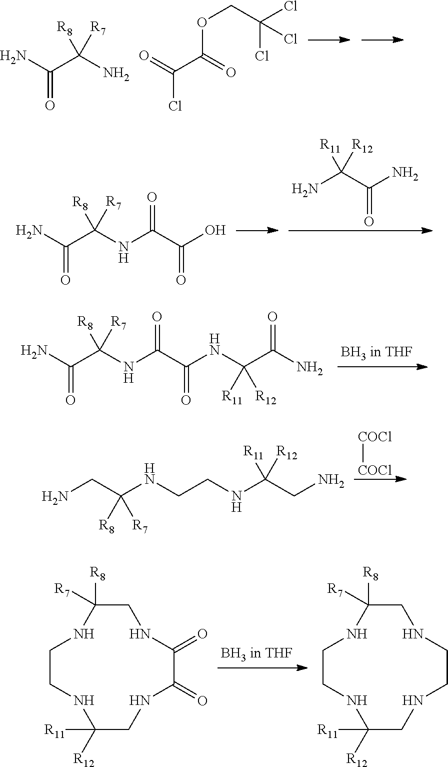

The oxalamide approach, shown above, also can lead to successful syntheses of this class of compounds. This particular variant makes use of the trichloroethyl ester group to protect one of the carbolxylic acid functions of oxalic acid but other protecting groups are also envisaged. Reaction of an amino acid amide derived from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative. Further reaction with oxalic acid gives the cyclic derivative, which can then be reduced once again with diborane to give the 12N4 series of compounds.
3NX series:
when X₁, X₂, X₃, are N and X₄ is S or O then:
R₅ does not exist;
R₂, R₃, and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, n3, and n4 are independently chosen to be 2 or 3, and each repeat of any of n1, n2, n3 and n4 may be the same as or different than any other repeat; and
R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃ or R₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 3NX Series of Formula I

Triethylenetetramine itself has been synthesized by reaction of 2 equivalents of ethylene diamine with 1,2-dichloro ethane to give triethylenetetramine directly (1). Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate R groups leads to symmetrically substituted 12N4 macrocycle examples as shown below:

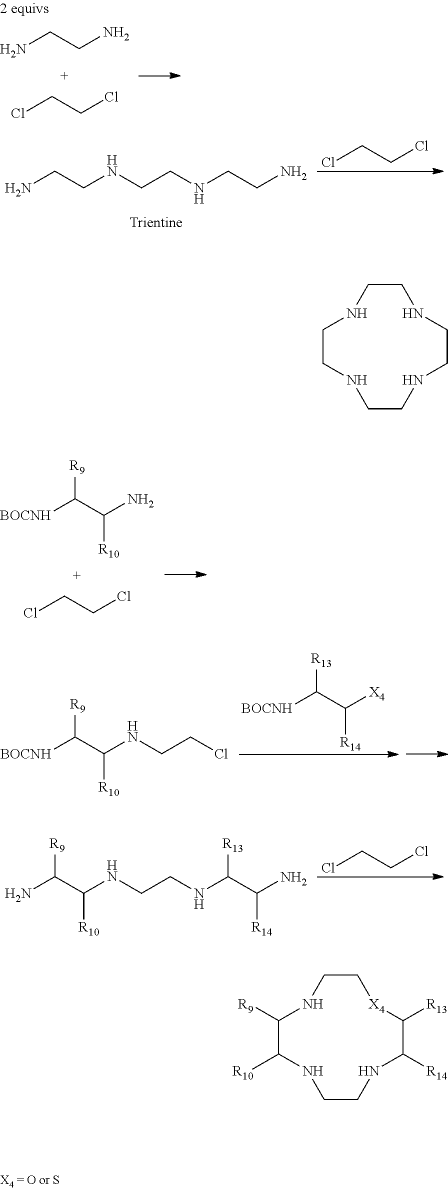

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tri-aza X series. In order to obtain alternative un-symmetrically substituted derivatives a variant of some chemistry described by Meares et al (3) could be used. Standard peptide synthesis using the Merrifield approach or the SASRIN resin along with FMOC protected natural and un-natural amino acids which can be conveniently cleaved at a later step of the synthesis generates a tri-peptide C-terminal SASRIN derivative which can be further elaborated with an appropriate BOCO or BOCS compound the give the resin bound 3NX compound shown. Reduction with diborane followed by Tosylation would give the 3NX OTosyl linear compound, which, upon deprotection and cyclization would give the desired 3NX macrocycle as shown below:

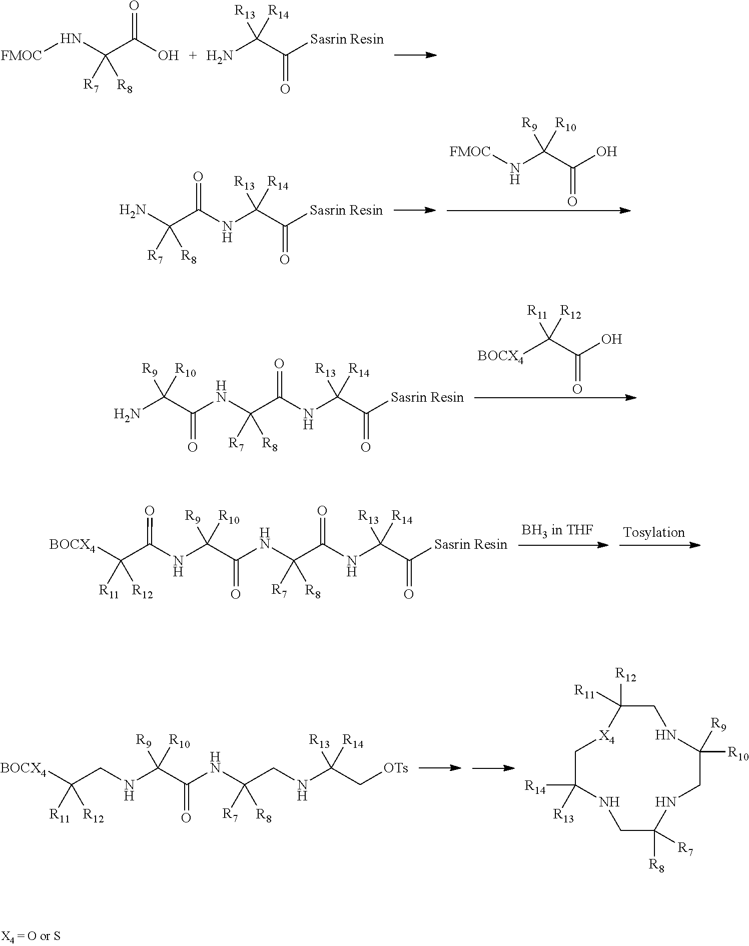

The incorporation of R₁, R₂, R₅ and R₆ can be accomplished with this chemistry by standard procedures.

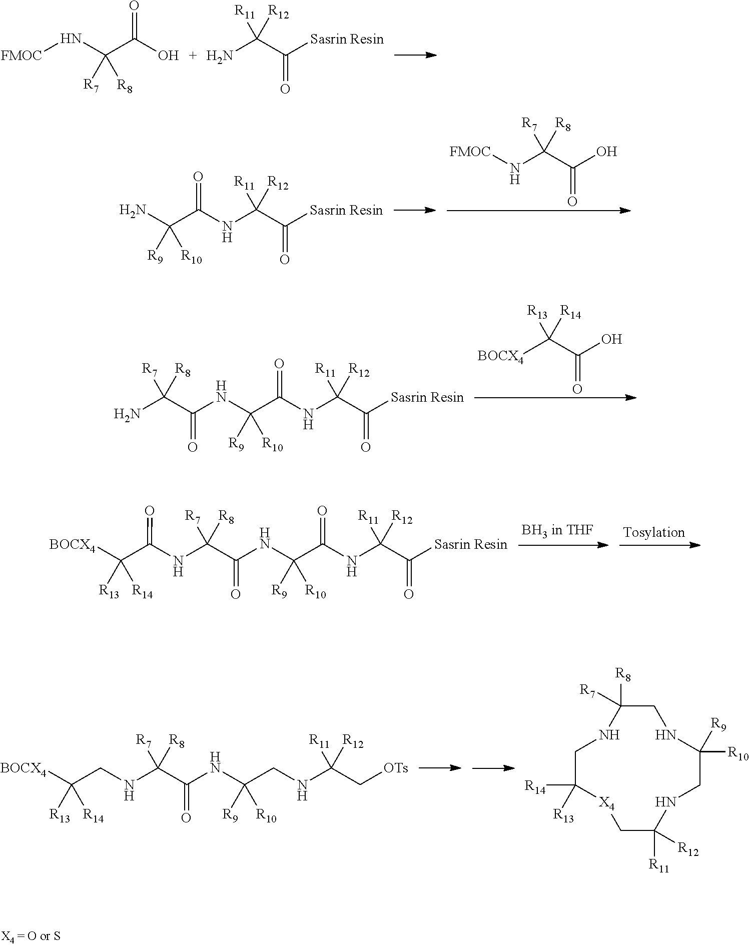

The reverse Merrifield/SASRIN approach, shown above, also leads to this class of tetra-aza derivatives and may be useful in cases where peptide coupling of a sterically hindered amino acid requires multiple coupling attempts in order to achieve success in the initial Merrifield approach.
2N2X series 1:
when X₂ and X₃ are N and X₁ and X₄ are O or S then:
R₂ and R₅ do not exist
R₃ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, n3, and n4 are independently chosen to be 2 or 3, and each repeat of any of n1, n2, n3 and n4 may be the same as or different than any other repeat; and
R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or both of R₃, or R₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 2N2X Series 1 of Formula I

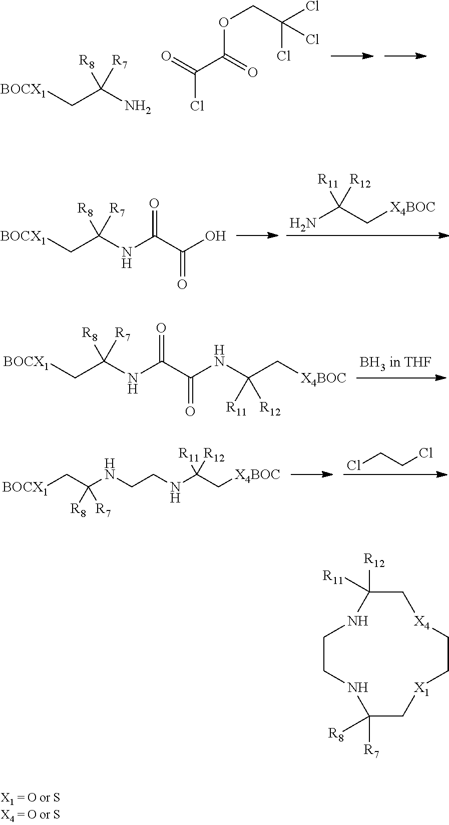

The oxalamide approach, shown above, again can lead to successful syntheses of this class of compounds, although the central substituents are always going to be hydrogen or its isotopes with this kind of chemistry. This particular variant makes use of the trichloroethyl ester group to protect one of the carboxylic acid functions of oxalic acid but other protecting groups are also envisaged. Reaction of an aminoalcohol or aminothiol derivative readily available from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired di-aza derivative. Deprotection followed by cyclization would give the 12N2X2 analogs.

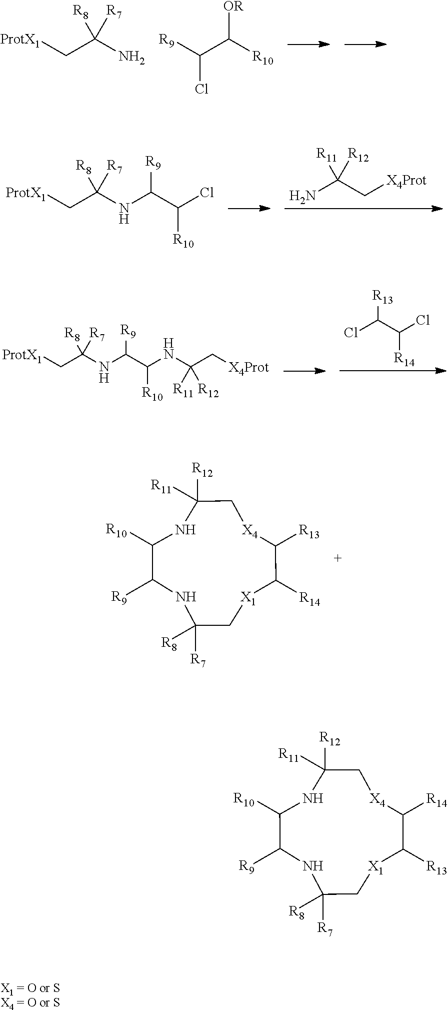

A variant of the dichloroethane approach, shown above, can also lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriate aminoalcohol or aminothiol derivative readily available from a natural or unnatural amino acid to give the un-symmetrical product shown. Deprotection followed by cyclization with a dichloroethane derivative would give a mixture of the the two position isomers shown.
2N2X series 2:
when X₁ and X₃ are N and X₂ and X₄ are O or S then:
R₃ and R₅ do not exist
R₂ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂2 PO(OH)₂, CH₂P(CH₃O(OH);
n1, n2, n3, and n4 are independently chosen to be 2 or 3, and each repeat of any of n1, n2, n3 and n4 may be the same as or different than any other repeat; and
R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or both of R₂, or R₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 2N2X Series 2 of Formula I

Triethylenetetramine itself has been synthesized by reaction of 2 equivalents of ethylene diamine with 1,2-dichloro ethane to give triethylenetetramine directly (1). Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate R groups would lead to symmetrically substituted 12N4 macrocycle examples as shown below:

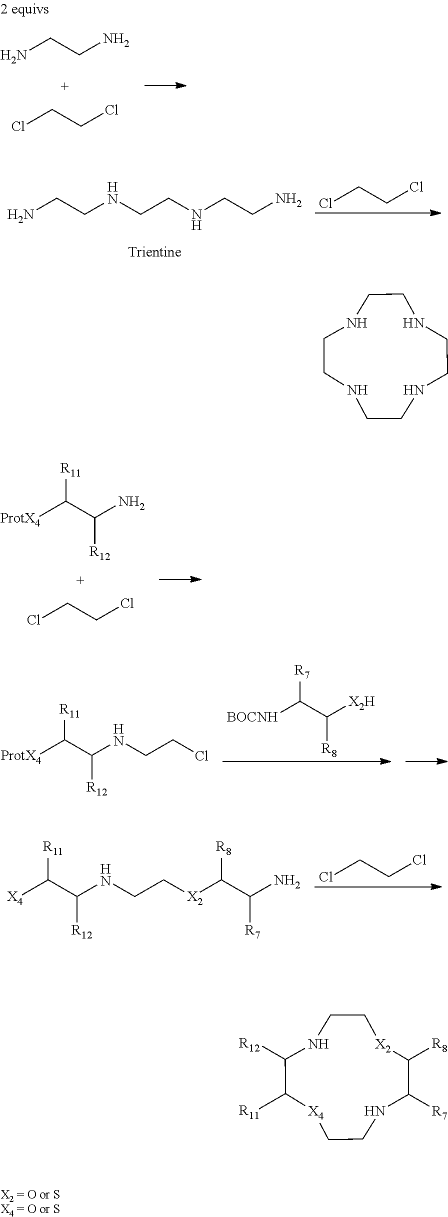

The judicious use of protecting group chemistry such as the widely used BOC (t-butyloxycarbonyl) group and an appropriate O or S protecting group allows the chemistry to be directed specifically towards the substitution pattern shown. Other approaches such as via the chemistry of ethyleneimine (2) may also lead to a subset of the di-aza 2X series. A variant of this approach using substituted dichloroethanee derivatives could be used to access more complex substitution patterns. This would lead to mixtures of position isomers, which can be separated by HPLC.

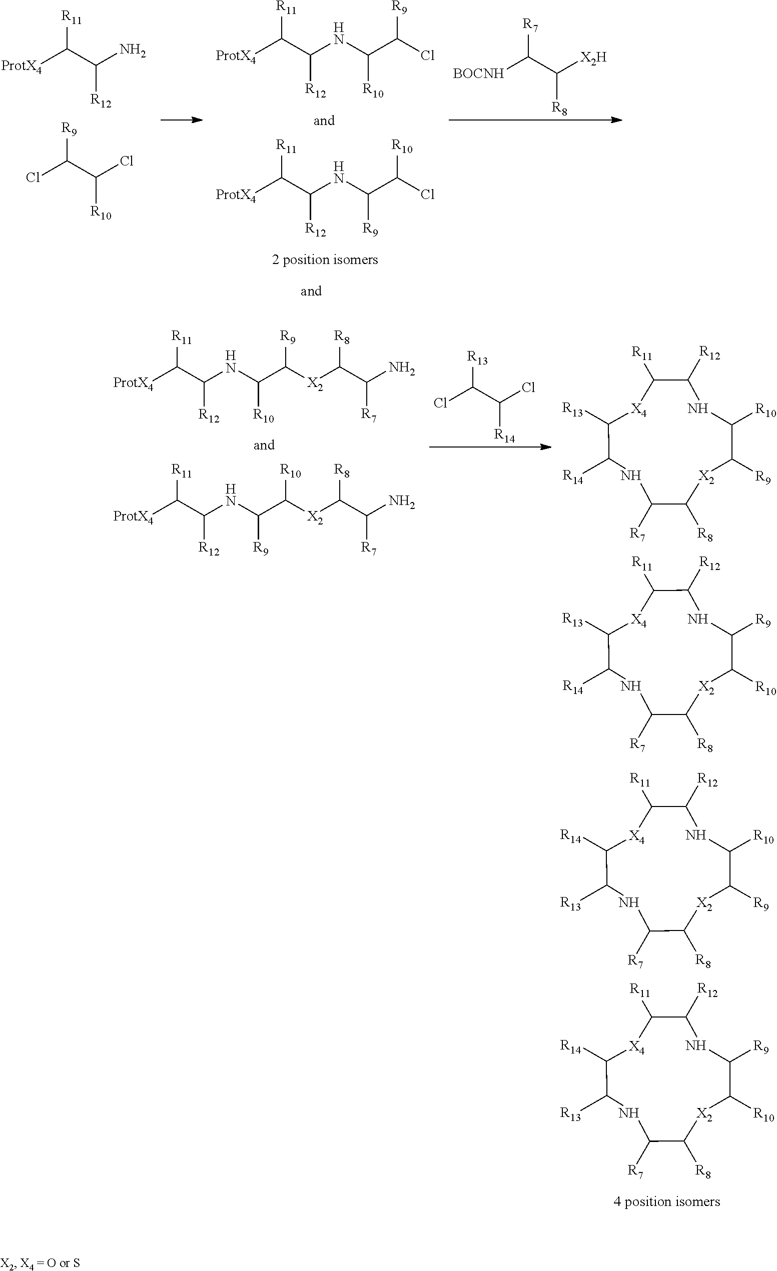

1N3X series:
when X₁ is N and X₂, X₃ and X₄ are O or S then:
R₃, R₄ and R₅ do not exist;
R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂ P(CH₃)O(OH);
n1, n2, n3, and n4 are independently chosen to be 2 or 3, and each repeat of any of n1, n2, n3 and n4 may be the same as or different than any other repeat; and
R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, R₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 1N3X Series of Formula I

Triethylenetetramine itself has been synthesized by reaction of 2 equivalents of ethylene diamine with 1,2-dichloro ethane to give triethylenetetramine directly (1). Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate R groups would lead to substituted 12NX3 macrocycle examples as shown below:

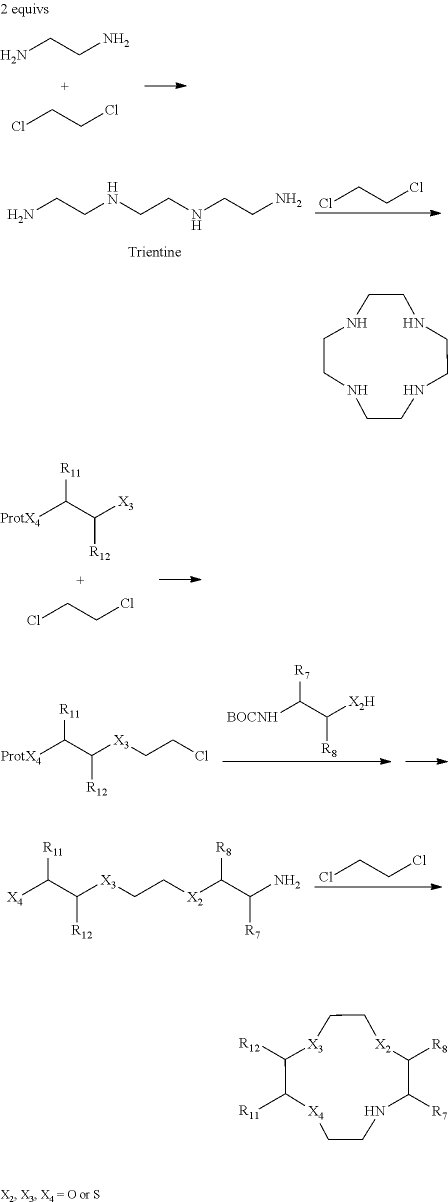

The judicious use of protecting group chemistry such as the widely used BOC (t-butyloxycarbonyl) group and an appropriate O or S protecting group allows the chemistry to be directed specifically towards the substitution pattern shown. Other approaches such as via the chemistry of ethyleneimine (2) may also lead to a subset of the mono-aza 3X series. A variant of this approach using substituted dichloroethane derivatives could be used to access more complex substitution patterns. This would lead to mixtures of position isomers, which can be separated by HPLC.

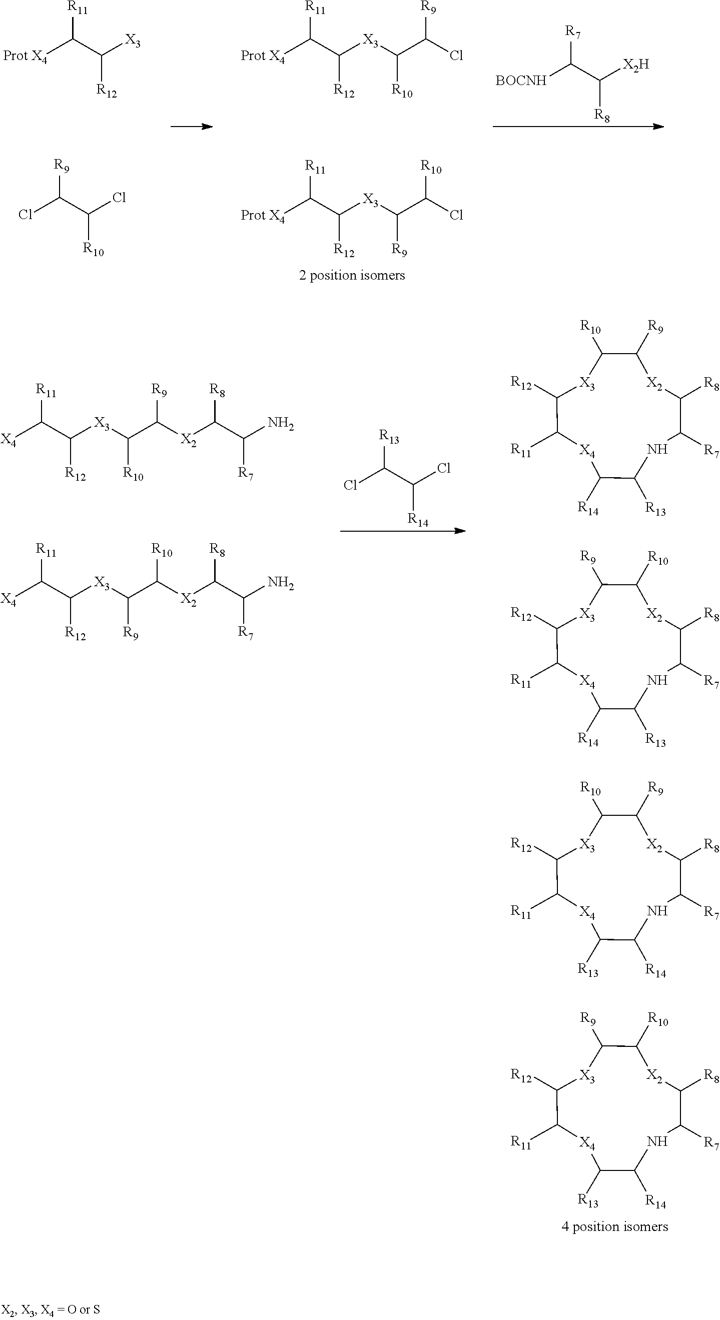

For the Tri-Heteroatom Acyclic Examples of Formula II:

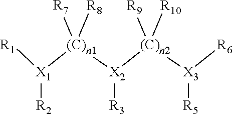

X₁, X₂, and X₃ are independently chosen from the atoms N, S or O such that:
3N series:
when X₁, X₂, and X₃ are N then:
R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1 and n2 are independently chosen to be 2 or 3, and each repeat of any of n1 and n2 may be the same as or different than any other repeat; and
R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₅ or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, or R₁₀ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 3N Series of Formula II

As mentioned above Triethylenetetramine itself has been synthesized by reaction of 2 equivalents of ethylene diamine with 1,2-dichloro ethane to give Triethylenetetramine directly (1). A variant of this procedure by using starting materials with appropriate R groups and 1-amino,2-chloro ethane would lead to some open chain 3N examples as shown below:

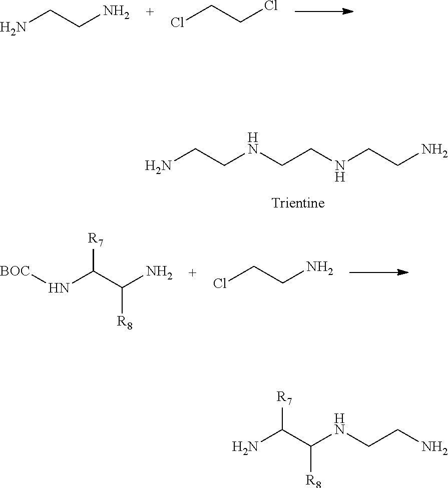

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the tri-aza series. In order to obtain the un-symmetrically substituted derivatives a variant of some chemistry described by Meares et al (2) could be used. Standard peptide synthesis using the Rink resin along with FMOC protected natural and un-natural amino acids which can be conveniently cleaved at the penultimate step of the synthesis generates a di-peptide C-terminal amide. This can be reduced using Diborane in THF to give the open chain tri-aza compounds as shown below:

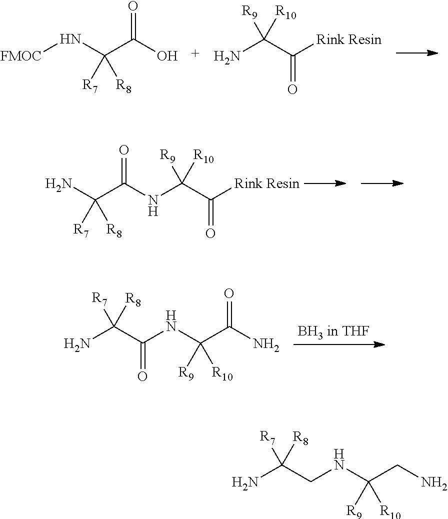

The reverse Rink approach may also be useful where peptide coupling is slowed for a particular substitution pattern as shown below. Again the incorporation of R₁, R₂, R₅ and R₆ can be accomplished with this chemistry by standard procedures:

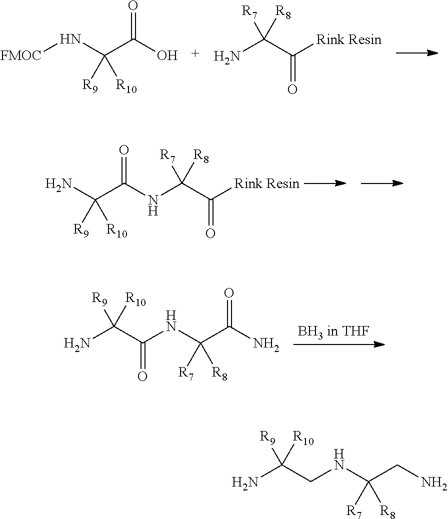

2NX series 1:
when X₁ and X₃ are N and X₂ is S or O then:
R₃ does not exist
R₁, R₂, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1 and n2 are independently chosen to be 2 or 3, and each repeat of any of n1 and n2 may be the same as or different than any other repeat; and
R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or several of R₁, R₂, R₅ or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, or R₁₀ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2NX Series 1 of Formula II

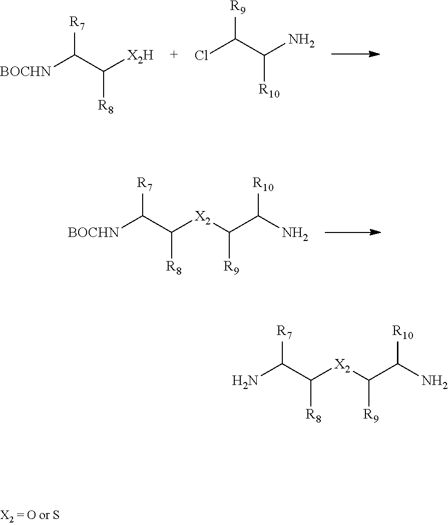

The synthesis of the 2NX series 1 compounds can be readily achieved as shown above. The judicious use of protecting group chemistry such as the widely used BOC (t-butyloxycarbonyl) group allows the chemistry to be directed specifically towards the substitution pattern shown above. Other approaches such as via the chemistry of ethyleneimine (2) may also lead to a subset of the tri-aza X series.
2NX series 2
when X₁ and X₂ are N and X₃ is O or S then:
R₅ does not exist;
R₁, R₂, R₃ and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1 and n2 are independently chosen to be 2 or 3, and each repeat of any of n1 and n2 may be the same as or different than any other repeat; and
R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, or R₁₀ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2NX Series 2 of Formula II

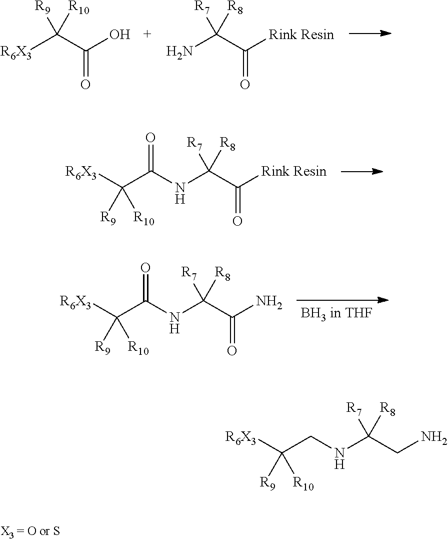

For the cases where X₃=0 or S a similar approach using standard peptide synthesis according to the Rink approach as shown above can be used. Coupling of a suitably protected alpha thiolo or hydroxy carboxylic acid with a Rink resin amino acid derivative followed by cleavage gives the desired linear di-amide, which can be reduced with Diborane in THF to give the open chain 2NX compounds.

The incorporation of R₁, R₂, R₅ and R₆ can be accomplished with this chemistry by standard procedures.

The reverse Rink version is also feasible and again the incorporation of R₁, R₂, R₅ and R₆ can be accomplished with this chemistry by standard procedures.

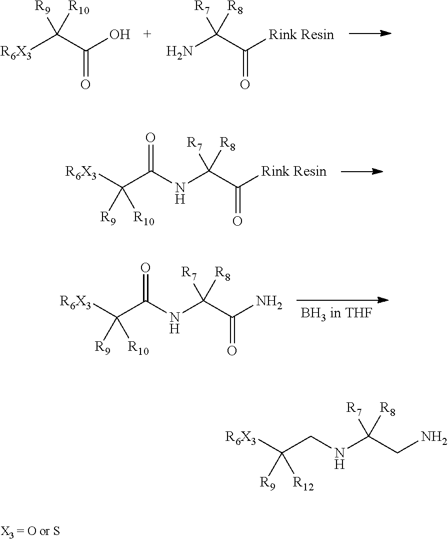

Tri-Heteroatom Cyclic Series of Formula II:
R₁ and R₆ form a bridging group (CR₁₁R₁₂)n3; and
X₁, X₂, and X₃ are independently chosen from the atoms N, S or O such that:
3N series:
when X₁, X₂ and X₃ are N then:
R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2 and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 3N Series of Formula II

As mentioned above Triethylenetetramine itself has been synthesized by reaction of 2 equivalents of ethylene diamine with 1,2-dichloro ethane to give Triethylenetetramine directly (1). A variant of this procedure by using starting materials with appropriate R groups and 1-amino,2-chloro ethane would lead to open chain 3N examples which could then be cyclized by reaction with an appropriate 1,2 dichloroethane derivative as shown below:

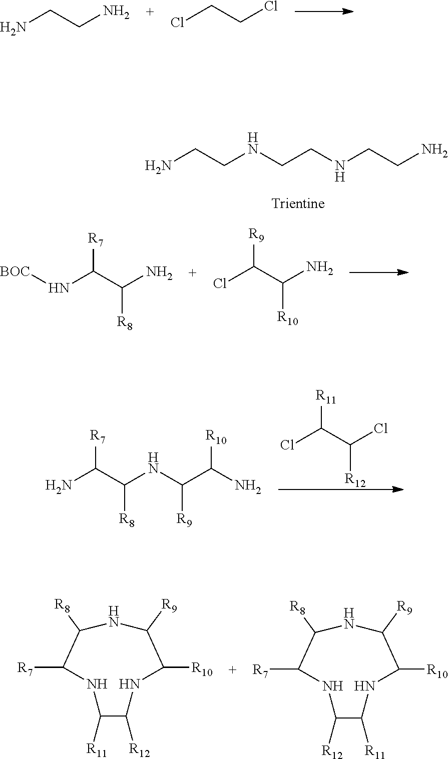

The judicious use of protecting group chemistry such as the widely used BOC (t-butyloxycarbonyl) group allows the chemistry to be directed specifically towards the substitution pattern shown. Other approaches such as via the chemistry of ethylenei mine (2) may also lead to a subset of the macrocyclic tri-aza series. In order to obtain the un-symmetrically substituted derivatives a variant of some chemistry described by Meares et al (3) could be used. Standard peptide synthesis using the Merrifield approach/SASRIN resin along with FMOC protected natural and un-natural amino acids which can be conveniently cleaved at the penultimate step of the synthesis generates a tri-peptide attached to resin via it's C-terminus. This can be cyclized during concomitant cleavage from the resin followed by reduction using Diborane in THF to give the cyclic tri-aza compounds as shown below:

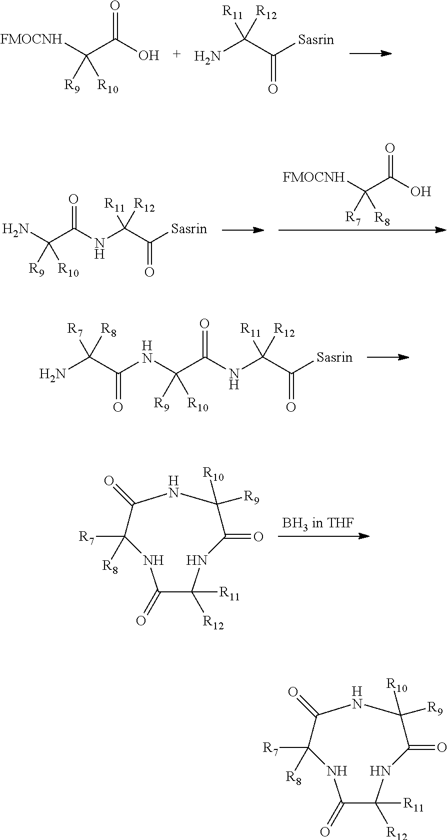

The incorporation of R₁, R₂, and R₅ can be accomplished with this chemistry by standard procedures.

The reverse Rink approach may also be useful where peptide coupling is slowed for a particular substitution pattern as shown below. Again the incorporation of R₁, R₂, R₅ and R₆ can be accomplished with this chemistry by standard procedures:

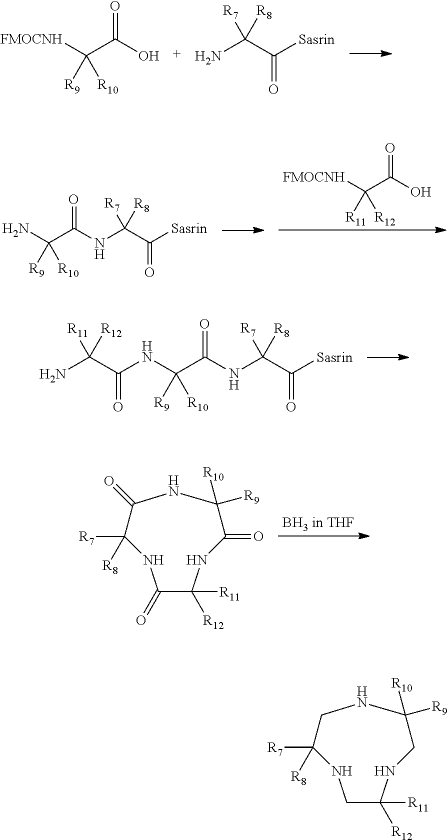

2NX series:
when X₁ and X₂ are N and X₃ is S or O then:
R₅ does not exist;
R₂ and R₃ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2 and n3 may be the same as or different than any other repeat; and
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or both of R₂ or R₃ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 2NX Series of Formula II

As mentioned above Triethylenetetramine itself has been synthesized by reaction of 2 equivalents of ethylene diamine with 1,2-dichloro ethane to give Triethylenetetramine directly (1). A variant of this procedure by using starting materials with appropriate R groups and 1-amino,2-chloro ethane would lead to open chain 2NX examples which could then be cyclized by reaction with an appropriate 1,2 dichloroethanee derivative as shown below:

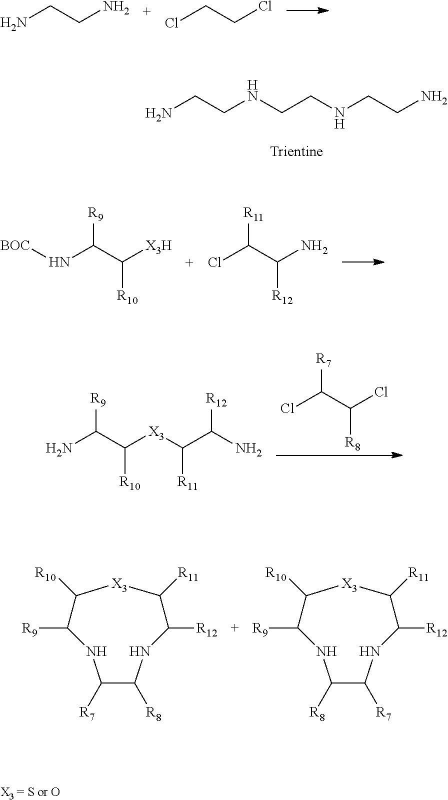

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the macrocyclic di-aza X series. In order to obtain the un-symmetrically substituted derivatives a variant of some chemistry described by Meares et al (3) could be used. Standard peptide synthesis using the Merrifield approach/SASRIN resin along with FMOC protected natural and un-natural amino acids which can be conveniently cleaved at the penultimate step of the synthesis generates a tri-peptide attached to resin via it's C-terminus. This can be cyclized during concomitant cleavage from the resin followed by reduction using Diborane in THF to give the cyclic tri-aza compounds as shown below:

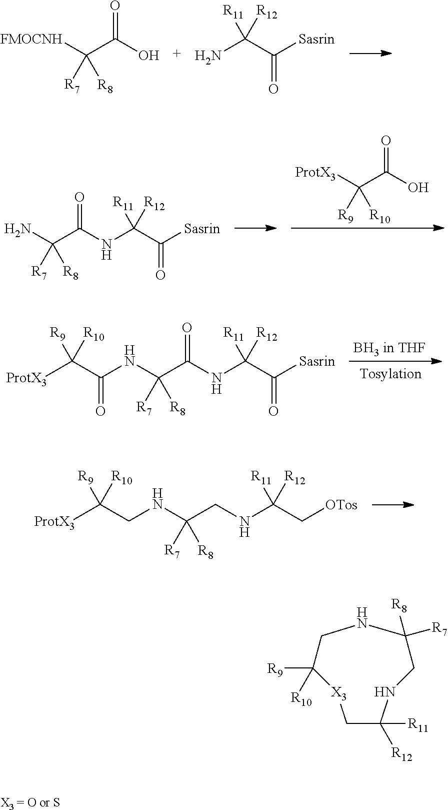

The incorporation of R₁, and R₂ can be accomplished with this chemistry by standard procedures.

The reverse Rink approach may also be useful where peptide coupling is slowed for a particular substitution pattern as shown below. Again the incorporation of R₁, and R₂ can be accomplished with this chemistry by standard procedures:

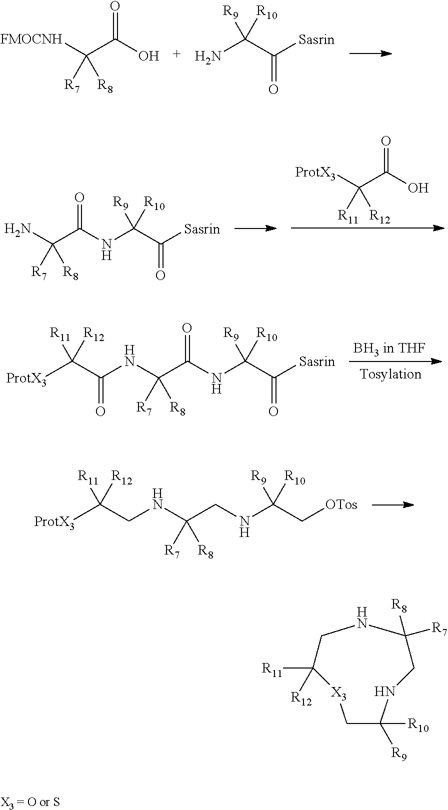

1N2X series:
when X₁ is N and X₂ and X₃ are O or S then:
R₃ and R₅ do not exist;
R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);
n1, n2, and n3 are independently chosen to be 2 or 3, and each repeat of any of n1, n2 and n3 may be the same as or different than any other repeat;
R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, R₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 1N2X Series of Formula II

As mentioned above Triethylenetetramine itself has been synthesized by reaction of 2 equivalents of ethylene diamine with 1,2-dichloro ethane to give Triethylenetetramine directly (1). A variant of this procedure by using starting materials with appropriate R groups and 1-amino,2-chloro ethane would lead to open chain 1N2X examples which could then be cyclized by reaction with an appropriate 1,2 dichloroethanee derivative as shown below:

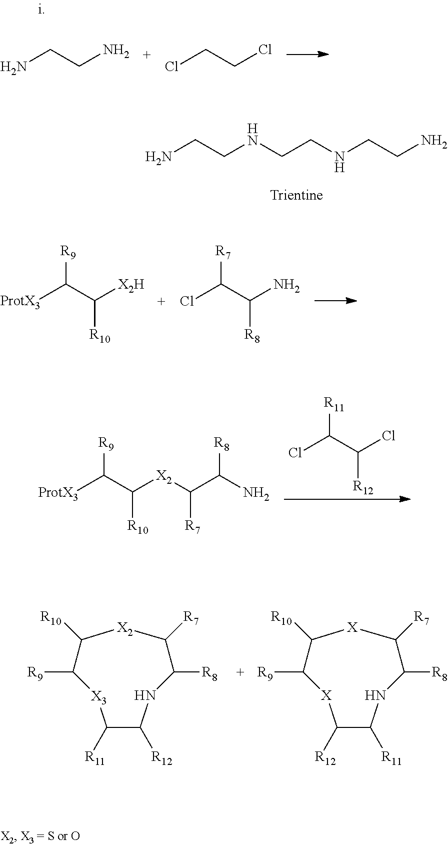

The judicious use of protecting group chemistry such as the widely used BOC (t-butyloxycarbonyl) group allows the chemistry to be directed specifically towards the substitution pattern shown. Other approaches such as via the chemistry of ethyleneimine (2) may also lead to a subset of the macrocyclic aza di-X series. In order to obtain the un-symmetrically substituted derivatives a variant of some chemistry above could be used:

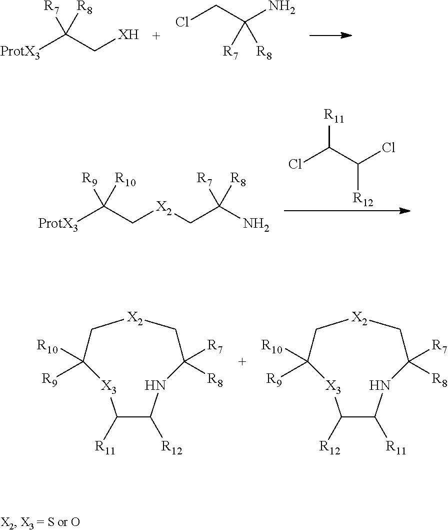

The incorporation of R₁ and R₂ can by accomplished with this chemistry by standard procedures.

Copper antagonists and pharmacutically acceptable salts of the invention may also be synthesized using methods decribed in U.S. patent application Ser. No. 11/184,761 filed Jul. 19, 2005, the contents of which are hereby incorporated by reference in its entirity.

Any of the methods of treating a subject having or suspected of having or predisposed to, or at risk for, a disease, disorder, and/or condition, referenced or described herein may utilize the administration of any of the doses, dosage forms, formulations, compositions and/or devices herein described.

Aspects of the invention include controlled or other doses, dosage forms, formulations, compositions and/or devices containing one or more copper antagonists, wherein the copper antagonists are, for example, one or more compounds of Formulae I or II and salts thereof, or other copper antagonists, for example, triethylenetetramine, triethylenetetramine disuccinate, triethylenetetramine di hydrochloride or other pharmaceutically acceptable salts. The present invention includes, for example, doses and dosage forms for at least oral administration, transdermal delivery, topical application, suppository delivery, transmucosal delivery, injection (including subcutaneous administration, subdermal administration, intramuscular administration, depot administration, and intravenous administration (including delivery via bolus, slow intravenous injection, and intravenous drip), infusion devices (including implantable infusion devices, both active and passive), administration by inhalation or insufflation, buccal administration, sublingual administration, and ophthalmic administration.

The invention includes, for example, methods for treating a subject having or suspected of having or predisposed to, or at risk for, any diseases, disorders and/or conditions characterized in whole or in part by an increase in mitochondrial number, an increase in mitochondrial protein expression, an increase in expression of nuclear mitochondrial genes, and/or an increase in mitochondrial swelling.

The invention also includes methods for treating a subject having or suspected of having or predisposed to, or at risk for, any diseases, disorders and/or conditions characterized in whole or in part by an increase in TGFβ-1 levels.

The invention further includes methods for treating a subject having or suspected of having or predisposed to, or at risk for, any diseases, disorders and/or conditions characterized in whole or in part by a decrease in Cu⁺¹ levels. Surprisingly, copper (II) antagonists, for example copper (II) chleators, that remove copper (II) serve to increase copper (I).

Diseases and disorders contemplated by the methods of treatment disclosed herein include, by way of example and not limitation, auto-immune disease, Alpers Disease (progressive infantile poliodystrophy, Barth syndrome, congenital muscular dystrophy, fatal infantile myopathy, “later-onset” myopathy, MELAS (mitochondrial encephalopathy, lactic acidosis, and stroke), MIDD (mitochondrial diabetes and deafness), MERRF (myoclonic epilepsy ragged red fiber syndrome), arthritis, NARP (Neuropathy; Ataxia; Retinitis Pigmentosa), MNGIE (Myopathy and external ophthalmoplegia; Neuropathy; Gastro-Intestinal; Encephalopathy), LHON (Leber's; Hereditary; Optic; Neuropathy), Kearns-Sayre disease, Pearson's Syndrome, PEO (Progressive External Ophthalmoplegia), Wolfram syndrome, DIDMOAD (Diabetes Insipidus, Diabetes Mellitus, Optic Atrophy, Deafness), ADPD (Alzheimer's disease; Parkinson's disease), AMFD (ataxia, myoclonus and deafness), CIPO (chronic intestinal pseudoobstruction; myopathy; opthalmoplegia), CPEO (chronic progressive external opthalmoplegia), maternally inherited deafness, aminoglycoside-induced deafness, DEMCHO (dementia; chorea), DMDF (diabetes mellitus; deafness), exercise intolerance, ESOC (epilepsy; strokes; optic atrophy; congenitive decline), FBSN (familial bilateral striatal necrosis), FICP (fatal infantile cardiomyopathy plus a MELAS-associated cardiomyopathy), GER (gastrointestinal reflux), LCHAD (Long-Chain Hydroxyacyl-CoA Dehydrogenase Deficiency), SCHAD (Sharot-Chain Hydroxyacyl-CoA Dehydrogenase Deficiency), MAD (Multiple Acyl-CoA Dehydrogenase Deficiency) MCAD (Medium-Chain Acyl-CoA Dehydrogenase Deficiency), SCAD (Short-Chain Acyl-CoA Dehydrogenase Deficiency), VLCAD (very long-chain Acyl-CoA Dehydrogenase Deficiency),_LIMM (lethal infantile mitochondrial myopathy), LDYT (Leber's hereditary optic neuropathy and DYsTonia), Luft Disease, MDM (myopathy; diabetes mellitus), MEPR (myoclonic epilepsy; psychomotor regression), MERME (MERRF/MELAS overlap disease), MHCM (maternally inherited hypertrophic cardiomyopathy), MICM (maternally inherited cardiomyopathy), MILS (maternally inherited Leigh syndrome), mitochondrial encephalocardiomyopathy, mitochondrial encephalomyopathy, mitochondrial myopathy, MMC (maternal myopathy; cardio myopathy), multisystem mitochondrial disorder (myopathy; encephalopathy; blindness; hearing loss; peripheral neuropathy), NIDDM (non-insulin dependent diabetes mellitus), Pearson Syndrome PEM (progressive encephalopathy), PME (progressive myclonus epilepsy), Rett syndrome, SIDS (sudden infant death syndrome, SNHL (sensorineural hearing loss), Leigh's Syndrome, dystonia, schizophrenia, and psoriasis..

Thus, the invention also is directed to doses, dosage forms, formulations, compositions and/or devices comprising one or more pharmaceutically acceptable copper antagonists, including those disclosed herein, useful for the therapy of diseases, disorders, and/or conditions in humans and other mammals and other disorders as disclosed herein. The use of these dosage forms, formulations compositions and/or devices of copper antagonist enables effective treatment of these conditions. The invention provides, for example, dosage forms, formulations, devices and/or compositions containing one or more copper antagonists, wherein the copper antagonists are, for example, copper chelators, such as copper (II) chelators. The dosage forms, formulations, devices and/or compositions of the invention may be formulated to optimize bioavailability and to maintain plasma concentrations within the therapeutic range, including for extended periods, and results in increases in the time that plasma concentrations of the copper antagonist(s) remain within a desired therapeutic range at the site or sites of action. Controlled delivery preparations also optimize the drug concentration at the site of action and minimize periods of under and over medication, for example.

The dosage forms, formulations, devices and/or compositions of the invention may be formulated for periodic administration, including once daily administration, to provide low dose controlled and/or low dose long-lasting in vivo release of a copper antagonist, wherein the copper antagonist is, for example, a copper chelator for chelation of copper and excretion of copper via the urine and/or to provide enhanced bioavailability of a copper antagonist, such as a copper chelator for chelation of copper and excretion of copper via the urine.

A therapeutically effective amount of a copper antagonist, for example a copper chelator, including but not limited to trientine, trientine salts, trientine analogues of formulae I and II, and so on, is from about 1 mg/kg to about 1 g/kg. Other therapeutically effective dose ranges include, for example, from about 1.5 mg/kg to about 950 mg/kg, about 2 mg/kg to about 900 mg/kg, about 3 mg/kg to about 850 mg/kg, about 4 mg/kg to about 800 mg/kg, about 5 mg/kg to about 750 mg/kg, about 5 mg/kg to about 700 mg/kg, 5 mg/kg to about 600 mg/kg, about 5 mg/kg to about 500 mg/kg, about 10 mg/kg to about 400 mg/kg, about 10 mg/kg to about 300 mg/kg, about 10 mg/kg to about 200 mg/kg, about 10 mg/kg to about 250 mg/kg, about 10 mg/kg to about 200 mg/kg, about 10 mg/kg to about 200 mg/kg, about 10 mg/kg to about 150 mg/kg, about 10 mg/kg to about 100 mg/kg, about 10 mg/kg to about 75 mg/kg, about 10 mg/kg to about 50 mg/kg, or about 15 mg/kg to about 35 mg/kg.

In some embodiments of the invention, a therapeutically effective amount of a copper antagonist (including, for example, a copper chelator, preferably a Cu⁺² binding agent or chelator), for example, trientine active agents, including but not limited to trientine, trientine salts, trientine analogues of formulae I and II, and so on, is from about 10 mg to about 4 g per day. Other therapeutically effective dose ranges include, for example, from about 20 mg to about 3.9 g, from about 30 mg to about 3.7 g, from about 40 mg to about 3.5 g, from about 50 mg to about 3 g, from about 60 mg to about 2.8 g, from about 70 mg to about 2.5 g, about 80 mg to about 2.3 g, about 100 mg to about 2 g, about 100 mg to about 1.5 g, about 200 mg to about 1400 mg, about 200 mg to about 1300 mg, about 200 mg to about 1200 mg, about 200 mg to about 1100 mg, about 200 mg to about 1000 mg, about 300 mg to about 900 mg, about 300 mg to about 800, about 300 mg to about 700 mg or about 300 mg to about 600 mg per day.

Copper antagonists (including precomplexed copper antagonists and pentacoordinate copper antagonist complexes), including but not limited to trientine active agents and compounds of Formulae I and II, and the like, will also be effective at doses in the order of 1/10, 1/50, 1/100, 1/200, 1/300, 1/400, 1/500 and even 1/1000 of those described herein.

The invention accordingly in part provides low dose compositions, formulations and devices comprising one or more copper antagonists. For example, low dose copper antagonists may include compounds, including copper chelators, particularly Cu+2 chelators, including but not limited to trientine active agents and compounds of Formulae I and II, and the like, in an amount sufficient to provide, for example, dosages from about 0.001 mg/kg to about 5 mg/kg, about 0.01 mg/kg to about 4.5 mg/kg, about 0.02 mg/kg to about 4 mg/kg, about 0.02 to about 3.5 mg/kg, about 0.02 mg/kg to about 3 mg/kg, about 0.05 mg/kg to about 2.5 mg/kg, about 0.05 mg/kg to about 2 mg/kg, about 0.05-0.1 mg/kg to about 5 mg/kg, about 0.05-0.1 mg/kg to about 4 mg/kg, about 0.05-0.1 mg/kg to about 3 mg/kg, about 0.05-0.1 mg/kg to about 2 mg/kg, about 0.05-0.1 mg/kg to about 1 mg/kg, and/or any other doses or dose ranges within the ranges set forth herein.

In some embodiments of the invention, a therapeutically effective amount is an amount effective to elicit a plasma concentration of a copper antagonist, for example, a copper chelator, including for example, trientine active agents, including but not limited to trientine, trientine salts, and compounds of formulae I and II, and so on, from about 0.01 mg/L to about 20 mg/L, about 0.01 mg/L to about 15 mg/L, about 0.1 mg/L to about 10 mg/L, about 0.5 mg/L to about 9 mg/L, about 1 mg/L to about 8 mg/L, about 2 mg/L to about 7 mg/L or about 3 mg/L to about 6 mg/L.

The doses described herein, may be administered in a single dose or multiple doses. For example, doses may be administered, once, twice, three, four or more times a day.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a copper antagonist.

Examples of dosage forms suitable for transdermal administration include, but are not limited, to transdermal patches, transdermal bandages, and the like. Examples of dosage forms suitable for topical administration of the compounds and formulations of the invention are any lotion, stick, spray, ointment, paste, cream, gel, etc., whether applied directly to the skin or via an intermediary such as a pad, patch or the like.

Examples of dosage forms suitable for suppository administration of the compounds and formulations of the invention include any solid dosage form inserted into a bodily orifice particularly those inserted rectally, vaginally and urethrally.

Examples of dosage forms suitable for transmucosal delivery of the compounds and formulations of the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations of the invention include delivery via bolus such as single or multiple administrations by intravenous injection, subcutaneous, subdermal, and intramuscular administration or oral administration.

Examples of dosage forms suitable for depot administration of the compounds and formulations of the invention include pellets or small cylinders of active agent or solid forms wherein the active agent is entrapped in a matrix of biodegradable polymers, microemulsions, liposomes or is microencapsulated.

Examples of infusion devices for compounds and formulations of the invention include infusion pumps containing one or more copper antagonists at a desired amount for a desired number of doses or steady state administration, and include implantable drug pumps.

Examples of implantable infusion devices for compounds, and formulations of the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of the compounds and formulations of the invention include compositions comprising solutions and/or suspensions in pharmaceutically acceptable, aqueous, or organic solvents, or mixture thereof and/or powders.

Examples of dosage forms suitable for buccal administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for opthalmic administration of the compounds and formulations of the invention include inserts and/or compositions comprising solutions and/or suspensions in pharmaceutically acceptable, aqueous, or organic solvents.

Examples of controlled drug formulations useful for delivery of the compounds and formulations of the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp.. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

Further examples of dosage forms of the invention include, but are not limited to modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. Bioavailability and bioequivalence of extended-release oral dosage forms. U.S. Pharmacist 22 (Suppl.):3-12 (1997); Scale-up of oral extended-release drug delivery systems: part I, an overview. Pharmaceutical Manufacturing 2:23-27 (1985).

Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. Bioavailability and bioequivalence of extended-release oral dosage forms. US Pharmacist 22 (Suppl.):3-12 (1997); Guidance for industry. Extended release oral dosage forms: development, evaluation, and application of the in vitro/in vivo correlations. Rockville, Md.: Center for Drug Evaluation and Research, Food and Drug Administration (1997).

Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner.

Targeted-release dosage forms of the invention include, for example, formulations that facilitate drug release and which are directed towards isolating or concentrating a drug in a body region, tissue, or site for absorption or for drug action.

The invention in part provides dosage forms, formulations, devices and/or compositions and/or methods utilizing administration of dosage forms, formulations, devices and/or compositions incorporating one or more copper antagonists complexed with one or more suitable anions to yield complexes that are only slowly soluble in body fluids. One such example of modified release forms of one or more copper antagonists is produced by the incorporation of the active agent or agents into certain complexes such as those formed with the anions of various forms of tannic acid (for example, see: Merck Index 12th Ed., 9221). Dissolution of such complexes may depend, for example, on the pH of the environment. This slow dissolution rate provides for the extended release of the copper antagonist. For example, salts of tannic acid, and/or tannates, provide for this quality, and are expected to possess utility for the treatment of conditions in which increased copper plays a role. Examples of equivalent products are provided by those having the tradename Rynatan (Wallace: see, for example, Madan, P. L., “Sustained release dosage forms,” U.S. Pharmacist 15:39-50 (1990); Ryna-12 S, which contains a mixture of mepyramine tannate with phenylephrine tannate, Martindale 33rd Ed., 2080.4).

Also included in the invention are coated beads, granules or microspheres containing one or more copper antagonists. Thus, the invention also provides a method to achieve modified release of one or more copper antagonists by incorporation of the drug into coated beads, granules, or microspheres. In such systems, the copper antagonist is distributed onto beads, pellets, granules or other particulate systems. See Ansel, H. C., Allen, L. V. and Popovich, N. G., Pharmaceutical Dosage Forms and Drug Delivery Systems, 7th Ed., Lippincott 1999, p. 232); Celphere microcrystalline cellulose spheres. Philadelphia: FMC Corporation, 1996). Methods for manufacture of microspheres suitable for drug delivery have been described. See, e.g., Arshady, R. Microspheres and microcapsules: a survey of manufacturing techniques. 1: suspension and cross-linking. Polymer Eng. Sci. 30:1746-1758 (1989); Arshady, R., Micro-spheres and microcapsules: a survey of manufacturing techniques. 2: coacervation. Polymer Eng Sci 30:905-914 (1990); Arshady R., Microspheres and microcapsules: a survey of manufacturing techniques. 3: solvent evaporation. Polymer Eng Sci 30:915-924 (1990)). In instances in which the copper antagonist dose is large, the starting granules of material may be composed of the copper antagonist itself. Some of these granules may remain uncoated to provide immediate copper antagonist release. Other granules (about two-thirds to three-quarters) receive varying coats of a lipid material such as beeswax, carnauba wax, glycerylmonostearate, cetyl alcohol, or a cellulose material such as ethylcellulose (infra). Subsequently, granules of different coating thickness are blended to achieve a mixture having the desired release characteristics. The coating material may be coloured with one or more dyes to distinguish granules or beads of different coating thickness (by depth of colour) and to provide distinctiveness to the product. When blended, the granules may be placed in capsules or tablets. Various coating systems are commercially available which are aqueous-based and which use ethylcellulose and plasticizer as the coating material. See, e.g., Aquacoat™ [FMC Corporation, Philadelphia] and Surerelease™ [Colorcon]; Aquacoat aqueous polymeric dispersion. Philadelphia: FMC Corporation, 1991; Surerelease aqueous controlled release coating system. West Point, Pa.: Colorcon, 1990; Butler, J., Cumming, I, Brown, J. et al., A novel multiunit controlled-release system, Pharm. Tech. 22:122-138 (1998); Yazici, E. et al., Phenytoin sodium microspheres: bench scale formulation, process characterization and release kinetics, Pharmaceut. Dev. Technol. 1:175-183 (1996)). See also Hogan, J. E. Aqueous versus organic solvent coating. Int. J. Pharm. Tech. Prod. Manufacture 3:17-20 (1982)). The variation in the thickness of the coats and in the type of coating materials used affects the rate at which the body fluids are capable of penetrating the coating to dissolve the copper antagonist. Typically, the coated beads are about 1 mm in diameter. They are usually combined to have three or four release groups among the more than 100 beads contained in the dosing unit. See Madan, P. L. Sustained release dosage forms. U.S. Pharmacist 15:39-50 (1990). This provides the different desired sustained or extended release rates and the targeting of the coated beads to the desired segments of the gastrointestinal tract. Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. The release rate can be controlled not only by incorporating therein suitable water-soluble pore formers, such as lactose, mannitol, sorbitol, etc., but also by the thickness of the coating layer applied. Multi-tablets may be formulated which include small spheroid-shaped compressed mini-tablets that may have a diameter of between 3 to 4 mm and can be placed in a gelatin capsule shell to provide the desired pattern of copper antagonist release. Each capsule may contain 8-10 minitablets, some uncoated for immediate release and others coated for extended release of the copper antagonist.

For orally administered dosage forms of the compounds and formulations of the invention, extended copper antagonist action, for example, copper chelator action, may be achieved by affecting the rate at which the copper antagonist is released from the dosage form and/or by slowing the transit time of the dosage form through the gastrointestinal tract. See Bogner, R. H., Bioavailability and bioequivalence of extended-release oral dosage forms. US Pharmacist 22 (Suppl.):3-12 (1997). The rate of drug release from solid dosage forms may be modified by the technologies described below which, in general, are based on the following: 1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the copper antagonist is either coated or entrapped in a substance that is slowly digested or dispersed into the intestinal tract. The rate of availability of the copper antagonist is a function of the rate of digestion of the dispersible material.

A further form of slow release dosage form of the compounds and formulations of the invention is any suitable osmotic system where semipermeable membranes of for example cellulose acetate, cellulose acetate butyrate, cellulose acetate propionate, is used to control the release of copper antagonist. These can be coated with aqueous dispersions of enteric lacquers without changing release rate. See, e.g., the Oros™ device developed by Alza Inc.

The invention also provides devices for compounds and formulations of the invention that utilize monolithic matrices including, for example, slowly eroding or hydrophilic polymer matrices, in which one or more copper antagonists are compressed or embedded. Monolithic matrix devices comprising compounds and formulations of the invention include those formed using, for example, hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Copper antagonist release occurs as the polymer swells, forming a matrix layer that controls the diffusion of aqueous fluid into the core and thus the rate of diffusion of copper antagonist from the system. In such systems, the rate of copper antagonist release depends upon the tortuous nature of the channels within the gel, and the viscosity of the entrapped fluid, such that different release kinetics can be achieved, for example, zero-order, or first-order combined with pulsatile release. Devices may contain 20-80% of copper antagonist (w/w), along with gel modifiers that can enhance copper antagonist diffusion; examples of such modifiers include sugars that can enhance the rate of hydration, ions that can influence the content of cross-links, and pH buffers that affect the level of polymer ionization. Hydrophilic matrix devices of the invention may also contain one or more pH buffers, surfactants, counter-ions, lubricants such as magnesium stearate (BP, USP) and a glidant such as colloidal silicon dioxide (USP; colloidal anhydrous silica, BP) in addition to copper antagonist and hydrophilic matrix; (II) copper antagonist particles are dissolved in an insoluble matrix, from which copper antagonist becomes available as solvent enters the matrix, often through channels, and dissolves the copper antagonist particles. Examples include systems formed with a lipid matrix, or insoluble polymer matrix, including preparations formed from Carnauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend of powdered components: lipids (20-40% hydrophobic solids w/w) which remain intact during the release process; copper antagonist, e.g., copper chelator; channeling agent, such as sodium chloride or sugars, which leaches from the formulation, forming aqueous micro-channels (capillaries) through which solvent enters, and through which copper antagonist is released. In the alternative system, which employs an insoluble polymer matrix, the copper antagonist is embedded in an inert insoluble polymer and is released by leaching of aqueous fluid, which diffuses into the core of the device through capillaries formed between particles, and from which the copper antagonist diffuses out of the device. The rate of release is controlled by the degree of compression, particle size, and the nature and relative content (w/w) of excipients. See, e.g., Bodmeier, R. and Paeratakul, O., “Drug release from laminated polymeric films prepared from aqueous latexes,” J. Pharm. Sci. 79:32-26 (1990); Laghoueg, N., et al., “Oral polymer-drug devices with a core and an erodible shell for constant drug delivery,” Int. J. Pharm. 50:133-139 (1989); Buckton, G., et al., “The influence of surfactants on drug release from acrylic matrices. Int. J. Pharm. 74:153-158 (1991)).

Further examples of monolithic matrix devices of the invention have compositions and formulations of the invention incorporated in pendent attachments to a polymer matrix. See, e.g., Scholsky, K. M. and Fitch, R. M., Controlled release of pendant bioactive materials from acrylic polymer colloids. J Controlled Release 3:87-108 (1986)). In these devices, copper antagonists, e.g., copper chelators, are attached by means of an ester linkage to poly(acrylate) ester latex particles prepared by aqueous emulsion polymerization. Yet further examples of monolithic matrix devices of the invention incorporate dosage forms of the compositions and formulations of the invention in which the copper antagonist is bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid. See Chafi, N., Montheard, J. P. and Vergnaud, J. M. Release of 2-aminothiazole from polymeric carriers. Int. J. Pharm. 67:265-274 (1992). See also Formulating for controlled release with Methocel Premium cellulose ethers. Midland, Mich.: Dow Chemical Company, 1995).

Two-layered tablets can be manufactured containing one or more of the compositions and formulations of the invention, with one layer containing the uncombined copper antagonist for immediate release and the other layer having the copper antagonist imbedded in a hydrophilic matrix for extended-release. Three-layered tablets may also be similarly prepared, with both outer layers containing the copper antagonist for immediate release. Some commercial tablets are prepared with an inner core containing the extended-release portion of drug and an outer shell enclosing the core and containing drug for immediate release.

The invention also provides forming a complex between the compositions and formulations of the invention and an ion exchange resin, whereupon the complex may be tableted, encapsulated or suspended in an aqueous vehicle. Alternative examples of this type of extended release preparation are provided by hydrocodone polistirex and chorpheniramine polistirex suspension (Medeva; Tussionex Pennkinetic Extended Release Suspension, see: Martindale 33rd Ed., p.2145.2) and by phentermine resin capsules (Pharmanex; Ionamin Capsules see: Martindale 33rd Ed., p.1916.1). Such preparations may also be suitable for administration, for example in depot preparations suitable for intramuscular injection.

The invention also provides a method to produce modified release preparations of one or more copper antagonists, wherein the copper antagonists are, for example, one or more copper chelators, by microencapsulation. See, e.g., U.S. Pat. Nos. 3,488,418; 3,391,416 and 3,155,590; Zentner, G. M., et al., Osmotic flow through controlled porosity films: an approach to delivery of water soluble compounds, J Controlled Release 2:217-229 (1985); Fites, A. L., Banker, G. S., and Smolen, V. F., Controlled drug release through polymeric films, J. Pharm. Sci. 59:610-613 (1970); Samuelov, Y., Donbrow, M., and Friedman, M., Sustained release of drugs from ethylcellulose-polyethylene glycol films and kinetics of drug release, J. Pharm. Sci. 68:325-329 (1979). See also Ansel, H. C., et al., Pharmaceutical Dosage Forms and Drug Delivery Systems, 7th Ed., Lippincott 1999, p. 233); Yazici, E., et al., Phenytoin sodium microspheres: bench scale formulation, process characterization and release kinetics. Pharmaceut. Dev. Technol. 1996; 1:175-183).

Other useful approaches include those in which the copper antagonist is incorporated into polymeric colloidal particles or microencapsulates (microparticles, microspheres or nanoparticles) in the form or reservoir and matrix devices (see: Douglas, S. J., et al., “Nanoparticles in drug delivery,” C. R. C. Crit. Rev. Therap. Drug. Carrier Syst. 3:233-261 (1987); Oppenheim, R. C., “Solid colloidal drug delivery systems: nanoparticles,” Int. J. Pharm. 8:217-234 (1981); Higuchi, T., “Mechanism of sustained action medication: theoretical analysis of rate of release of solid drugs dispersed in solid matrices,” J. Pharm. Sci. 52:1145-1149 (1963)).

The invention also includes repeat action tablets containing one or more copper antagonists, for example, one or more copper chelators. These are prepared so that an initial dose of the copper antagonist is released immediately followed later by a second dose. The tablets may be prepared with the immediate-release dose in the tablet's outer shell or coating with the second dose in the tablet's inner core, separated by a slowly permeable barrier coating. In general, the copper antagonist from the inner core is exposed to body fluids and released 4 to 6 hours after administration. Repeat action dosage forms are suitable for the administration of one or more copper antagonists for the indications noted herein.

The invention also includes delayed-release oral dosage forms containing one or more copper antagonists, for example, one or more copper chelators. The release of one or more copper antagonists, for example, one or more copper chelators, from an oral dosage form can be intentionally delayed until it reaches the intestine at least in part by way of, for example, enteric coating. Among the many agents used to enteric coat tablets and capsules known to those skilled in the art are fats including triglycerides, fatty acids, waxes, shellac, and cellulose acetate phthalate although further examples of enteric coated preparations can be found in the USP.

The invention also provides devices incorporating one or more copper antagonists, for example, one or more copper chelators, in a membrane-control system. Such devices comprise a rate-controlling membrane enclosing a copper antagonist reservoir. Following oral administration the membrane gradually becomes permeable to aqueous fluids, but does not erode or swell. The copper antagonist reservoir may be composed of a conventional tablet, or a microparticle pellet containing multiple units that do not swell following contact with aqueous fluids. The cores dissolve without modifying their internal osmotic pressure, thereby avoiding the risk of membrane rupture, and typically comprise 60:40 mixtures of lactulose: microcrystalline cellulose (w/w). Active drug(s) is/are released through a two-phase process, comprising diffusion of aqueous fluids into the matrix, followed by diffusion of the copper antagonist out of the matrix. Multiple-unit membrane-controlled systems typically comprise more than one discrete unit. They can contain discrete spherical beads individually coated with rate-controlling membrane and may be encapsulated in a hard gelatin shell. Alternatively, multiple-unit membrane-controlled systems may be compressed into a tablet. Alternative implementations of this technology include devices in which the copper antagonist is coated around inert sugar spheres, and devices prepared by extrusion spheronization employing a conventional matrix system.

An example of a sustained release dosage form of one or more compounds and formulations of the invention is a matrix formation, such a matrix formation taking the form of film coated spheroids containing as active ingredient one or more copper antagonists, for example, one or more copper chelators and a non water soluble spheronising agent. The term “spheroid” is known in the pharmaceutical art and means spherical granules having a diameter usually of between 0.01 mm and 4 mm. The spheronising agent may be any pharmaceutically acceptable material that, together with the copper antagonist, can be spheronised to form spheroids. Microcrystalline cellulose is preferred. Suitable microcrystalline cellulose includes, for example, the material sold as Avicel PH 101 (Trade Mark, FMC Corporation). The film-coated spheroids may contain between 70% and 99% (by wt), especially between 80% and 95% (by wt), of the spheronising agent, especially microcrystalline cellulose. In addition to the active ingredient and spheronising agent, the spheroids may also contain a binder. Suitable binders, such as low viscosity, water soluble polymers, will be well known to those skilled in the pharmaceutical art. A suitable binder is, in particular polyvinylpyrrolidone in various degrees of polymerization. However, water-soluble hydroxy lower alkyl celluloses, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose. Other thickening agents or binders include: the lipid type, among which are vegetable oils (cotton seed, sesame and groundnut oils) and derivatives of these oils (hydrogenated oils such as hydrogenated castor oil, glycerol behenate, the waxy type such as natural carnauba wax or natural beeswax, synthetic waxes such as cetyl ester waxes, the amphiphilic type such as polymers of ethylene oxide (polyoxyethylene glycol of high molecular weight between 4000 and 100000) or propylene and ethylene oxide copolymers (poloxamers), the cellulosic type (semisynthetic derivatives of cellulose, hydroxypropylmethylcellulose, hydroxypropylcellulose, hydroxymethylcellulose, of high molecular weight and high viscosity, gum) or any other polysaccharide such as alginic acid, the polymeric type such as acrylic acid polymers (such as carbomers), and the mineral type such as colloidal silica and bentonite.

Suitable diluents for the copper antagonist(s) in the pellets, spheroids or core are, e.g., microcrystalline cellulose, lactose, dicalcium phosphate, calcium carbonate, calcium sulphate, sucrose, dextrates, dextrin, dextrose, dicalcium phosphate di hydrate, kaolin, magnesium carbonate, magnesium oxide, maltodextrin, cellulose, microcrystalline cellulose, sorbitol, starches, pregelatinized starch, talc, tricalcium phosphate and lactose. Suitable lubricants are e.g., magnesium stearate and sodium stearyl fumarate. Suitable binding agents include, e.g., hydroxypropyl methylcellulose, polyvidone, and methylcellulose.

Suitable binders that may be included are: gum arabic, gum tragacanth, guar gum, alginic acid, sodium alginate, sodium carboxymethylcellulose, dextrin, gelatin, hydroxyethylcellulose, hydroxypropylcellulose, liquid glucose, magnesium and aluminum. Suitable disintegrating agents are starch, sodium starch glycolate, crospovidone and croscarmalose sodium. Suitable surface active are Poloxamer 188®, polysorbate 80 and sodium lauryl sulfate. Suitable flow aids are talc colloidal anhydrous silica. Suitable lubricants that may be used are glidants (such as anhydrous silicate, magnesium trisilicate, magnesium silicate, cellulose, starch, talc or tricalcium phosphate) or alternatively antifriction agents (such as calcium stearate, hydrogenated vegetable oils, paraffin, magnesium stearate, polyethylene glycol, sodium benzoate, sodium lauryl sulphate, fumaric acid, stearic acid or zinc stearate and talc). Suitable water-soluble polymers are PEG with molecular weights in the range 1000 to 6000.

Examples of lubricants and nonstick agents are higher fatty acids and their alkali metal and alkaline-earth-metal salts, such as calcium stearate. Suitable disintegrants are, in particular, chemically inert agents, for example, cross-linked polyvinylpyrrolidone, cross-linked sodium carboxymethylcelluloses, and sodium starch glycolate.

Yet further embodiments of the invention include formulations of one or more copper antagonists, for example, one or more copper chelators, incorporated into transdermal drug delivery systems, such as those described in: Transdermal Drug Delivery Systems, Chapter 10. In: Ansel, H. C., Allen, L. V. and Popovich, N. G. Pharmaceutical Dosage Forms and Drug Delivery Systems, 7th Ed., Lippincott 1999, pp. 263-278). Formulations of drugs suitable for trans-dermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion. See, e.g., Shah, V., Peck, C. C., and Williams, R. L., Skin penetration enhancement: clinical pharmacological and regulatory considerations, In: Walters, K. A. and Hadgraft, J. (Eds.) Pharmaceutical skin penetration enhancement. New York: Dekker, 1993); Osborne, D. W., and Henke, J. J., “Skin penetration enhancers cited in the technical literature,” Pharm. Tech. 21:50-66 (1997); Rolf, D., “Chemical and physical methods of enhancing transdermal drug delivery,” Pharm. Tech. 12:130-139 (1988)). In addition to chemical means, there are physical methods that enhance transdermal drug delivery and penetration of the compounds and formulations of the invention, including iontophoresis and sonophoresis. Accordingly, another embodiment of the invention comprises one or more copper antagonists, for example, one or more copper chelators, formulated in such a manner suitable for administration by iontophoresis or sonophoresis.

Formulations and/or compositions for topical administration of one or more compositions and formulations of the invention ingredient can be prepared as an admixture or other pharmaceutical formulation to be applied in a wide variety of ways including, but are not limited to, lotions, creams gels, sticks, sprays, ointments and pastes. These product types may comprise several types of formulations including, but not limited to solutions, emulsions, gels, solids, and liposomes. If the topical composition of the invention is formulated as an aerosol and applied to the skin as a spray-on, a propellant may be added to a solution composition. Suitable propellants as used in the art can be utilized. By way of example of topical administration of an active agent, reference is made to U.S. Pat. Nos. 5,602,125, 6,426,362 and 6,420,411.

Also included in the dosage forms in accordance with the present invention are any variants of the oral dosage forms that are adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquefy and/or dissolve in the rectal cavity to release the copper antagonist (e.g., chelator). Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7).

Transmucosal administration of the compounds and formulations of the invention may utilize any mucosal membrane but commonly utilizes the nasal, buccal, vaginal and rectal tissues. Formulations suitable for nasal administration of the compounds and formulations of the invention may be administered in a liquid form, for example, nasal spray, nasal drops, or by aerosol administration by nebulizer, including aqueous or oily solutions of the copper antagonist. Formulations for nasal administration, wherein the carrier is a solid, include a coarse powder having a particle size, for example, of less than about 100 microns, preferably less, most preferably one or two times per day than about 50 microns, which is administered in the manner in which snuff is taken, i.e., by rapid inhalation through the nasal passage from a container of the powder held close up to the nose. Formulations of the invention may be prepared as aqueous solutions for example in saline, solutions employing benzyl alcohol or other suitable preservatives, absorption promoters to enhance bio-availability, fluorocarbons, and/or other solubilising or dispersing agents known in the art.

The invention provides extended-release formulations containing one or more copper antagonists, for example, one or more copper chelators, for parenteral administration. Extended rates of copper antagonist action following injection may be achieved in a number of ways, including the following: crystal or amorphous copper antagonist forms having prolonged dissolution characteristics; slowly dissolving chemical complexes of the copper antagonist formulation; solutions or suspensions of copper antagonist in slowly absorbed carriers or vehicles (as oleaginous); increased particle size of copper antagonist in suspension; or, by injection of slowly eroding microspheres of copper antagonist. See, e.g., Friess, W., et al., Insoluble collagen matrices for prolonged delivery of proteins. Pharmaceut. Dev. Technol. 1:185-193 (1996).

Copper antagonists may be administered in a dose from between about 0.1 mg to about 1000 mg per day. In some embodiments, dosage forms of 100 mg, 200 mg, and 320 or 350 mg of a copper antagonist, for example, a copper chelator, are provided. By way of example only, the amount of copper antagonist, for example triethylenetetramine di hydrochloride or triethylenetetramine disuccinate may range from about 1 mg to about 750 mg or more (for example, about 1 mg, about 5 mg, about 10 mg, about 25 mg, about 50 mg, about 100 mg, about 125 mg, about 150 mg, about 200 mg, about 250 mg, about 300, about 320, about 350, about 400 mg, about 500 mg, about 600 mg, about 750 mg, about 800 mg, about 1000 mg, and about 1200 mg). Other amounts within these ranges may also be used and are specifically contemplated though each number in between is not expressly set out.

The copper antagonist can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations are isotonic or substantially isotonic to avoid irritation and pain at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to about 9.5 can enhance the stability of the parenteral formulation of the present invention. Other pH ranges, for example, include, about 5.5 to about 9.0, or about 6.0 to about 8.5, or about 6.5 to about 8.0, or, preferably, about 7.0 to about 7.5.

The buffer used in the practice of the present invention is selected from any of the following, for example, an acetate buffer, a phosphate buffer or glutamate buffer, the most preferred buffer being a phosphate buffer.

Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents.

A stabilizer may be included in the formulations of the invention, but will generally not be needed. If included, however, a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

Anti-microbial agents in bacteriostatic or fungistatic concentrations are generally added to preparations contained in multiple dose containers.

A preservative is, in the common pharmaceutical sense, a substance that prevents or inhibits microbial growth and may be added to a pharmaceutical formulation for this purpose to avoid consequent spoilage of the formulation by microorganisms. While the amount of the preservative is not great, it may nevertheless affect the overall stability of the copper antagonist. While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in “Remington's Pharmaceutical Sciences” as well as Avis et al., Pharmaceutical Dosage Forms: Parenteral Medications, Vol. 1 (1992). For these purposes, the copper antagonist may be administered parenterally (including subcutaneous injections, intravenous, intramuscular, intradermal injection or infusion techniques) or by inhalation spray in dosage unit formulations containing conventional non-toxic pharmaceutically acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant.

It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

It is possible that other ingredients may be present in the parenteral pharmaceutical formulation of the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Containers and kits are also a part of a composition and may be considered a component. Therefore, the selection of a container is based on a consideration of the composition of the container, as well as of the ingredients, and the treatment to which it will be subjected.

Regarding pharmaceutical formulations, see also, Pharmaceutical Dosage Forms: Parenteral Medications, Vol. 1, 2nd ed., Avis et al., Eds., Mercel Dekker, New York, N.Y. 1992.

The copper antagonist(s), such as, for example, a copper chelator(s), can also be administered in the form of a depot injection that may be formulated in such a manner as to permit a sustained release of the copper antagonist.

Also useful are implantable infusion devices for delivery of compositions and formulations of the invention. Implantable infusion devices may employ inert material such as biodegradable polymers listed above or synthetic silicones, for example, cylastic, silicone rubber or other polymers manufactured by the Dow-Corning Corporation. The polymer may be loaded with copper antagonist and any excipients. Implantable infusion devices may also comprise a coating of, or a portion of, a medical device wherein the coating comprises the polymer loaded with copper antagonist and any excipient. Such an implantable infusion device may be prepared as disclosed in U.S. Pat. No. 6,309,380 by coating the device with an in vivo biocompatible and biodegradable or bioabsorbable or bioerodibleerodible liquid or gel solution containing a polymer with the solution comprising a desired dosage amount of copper antagonist and any excipients. An implantable infusion device may also be prepared by the in situ formation of a copper antagonist containing solid matrix as disclosed in U.S. Pat. No. 6,120,789, herein incorporated in its entirety. Implantable infusion devices may be passive or active.

The invention also includes delayed-release ocular preparations containing one or more copper antagonists, for example, one or more copper chelators. Preparations of one or more copper antagonists, for example, one or more copper chelators, suitable for ocular administration to humans may be formulated using synthetic high molecular weight cross-linked polymers such as those of acrylic acid (e.g., Carbopol 940) or gellan gum (Gelrite; see, Merck Index 12th Ed., 4389), a compound that forms a gel upon contact with the precorneal tear film (e.g. as employed in Timoptic-XE by Merck, Inc.).

An increase in bioavailability of a copper antagonist may be achieved by complexation of copper antagonist with one or more bioavailability or absorption enhancing agents or in bioavailability or absorption enhancing formulations. Such bioavailability or absorption enhancing agents include, but are not limited to, various surfactants such as various triglycerides, such as from butter oil, monoglycerides, such as of stearic acid and vegetable oils, esters thereof, esters of fatty acids, propylene glycol esters, the polysorbates, sodium lauryl sulfate, sorbitan esters, sodium sulfosuccinate, among other compounds. The invention in part also provides for the formulation of copper antagonist, e.g., a copper chelator, in a microemulsion to enhance bioavailability. A microemulsion is a fluid and stable homogeneous solution composed of four major constituents, respectively, a hydrophilic phase, a lipophilic phase, at least one surfactant (SA) and at least one cosurfactant (CoSA).

A better understanding of the invention will be gained by reference to the following experimental section. The following experiments are illustrative and are not intended to limit the invention or the claims in any way.

All the following experiments were performed under the appropriate approvals(s) from the University of Auckland Animal Ethics Committee.

### Example 1

### Protein Induced X-Ray Emission Microscopy (Pixe) of Left Ventricle Wall

Male Wister rats (starting body weight from about 220 g to about 250 g) were maintained on Teklad TB 2108 (Harlan UK) rat chow and tap water ab libitum. Animals were randomized into two groups: Sham-control and diabetic (STZ). The animals were anesthetized by halthane inhalation (2%-5% halothane and 2 L/min oxygen). Rats were made diabetic by injection with 60 mg/kg streptozocin, while the control rats were given a corresponding amount of 0.9% sodium chloride. Blood glucose levels and body weight were measured 3 days post injection and once a week thereafter. Glucose levels were measured using the Advantage II system (Roche Diagnostics). Animals with recurrent glucose levels greater than 11 mM were considered to have established diabetes.

Rats receiving STZ were randomized into two groups: one group received triethylenetetramine dihydrochloride treatment (T-STZ) and the second group did not receive triethylenetetramine dihydrochloride treatment (STZ).

Triethylenetetramine dihydrochloride was administered to T-STZ diabetic rats via drinking water at a dose of about 10 mg/day. Water intake per day was calculated for each cage and averaged over the week. This data was then used to calculate the appropriate concentration of drug to be added to the drinking water for the subsequent week. Treatment began at the start of week nine after STZ injection and continued for eight weeks. At the end of the eight week treatment, animals were anesthetized by halothane inhalation 2% -5% in oxygen. The chest was opened and the heart was rapidly removed and rinsed in 0.9% saline solution. The heart was dissected and the LV was frozen in cryomold, floated in liquid nitrogen cooled isopentane and stored at −70° C.

The LV cardiac tissue was mounted on formvar film for PIXE analysis. A 100 nm formvar film was made on an aluminium target holder having a 10 mm diameter aperture. The film was produced by placing a drop of 1% formvar solution (Sigma) (dissolved in 1,2-dichloroethane) onto the surface of milliQ water, forming a sheet of formvar. The aluminium target holder was submerged in the water and brought out through the film, such that the aperture was completely covered with the film. The holder was then placed in an oven at 45° C. for 60 minutes to dry the film. 20 μm cryostat cross-sections of the LV and aorta were thaw mounted onto room temperature formvar film mounts. The mounts were allowed to dry and then stored at −30° C. under dessicant.

PIXE analysis was performed at the Institute of Geological and Nuclear Sciences. Tissue samples were mounted in a vacuum chamber (10⁻⁶ MBAR). Microprobe analysis was performed using a 2 MeV proton beam, generated by the 3 MV KN van de Graaff accelerator. Measurements were taken for approximately 30 minutes on each sample, with a beam spot around 15 μm and a current of 0.5 nÅ. Rutherford Back Scattering Spectroscopy (RBS) was simultaneously employed to determine the bulk elemental content and the organic mass of the analyzed tissues. A Scanning Transmission Ion Microscopy (STIM) image was also generated to probe the tissue structure and density. Elemental concentrations in ng/cm² were extracted using GUPIX software (http://pixie.physics.uoguelph.ca/gupix/main/2004 version). The area mass of the tissues was then calculated using RBS and STIM data and expressed in dry weight (g/cm²). This information was then used to calculate quantitative results normalized in terms of mass (μg/g dry weight).

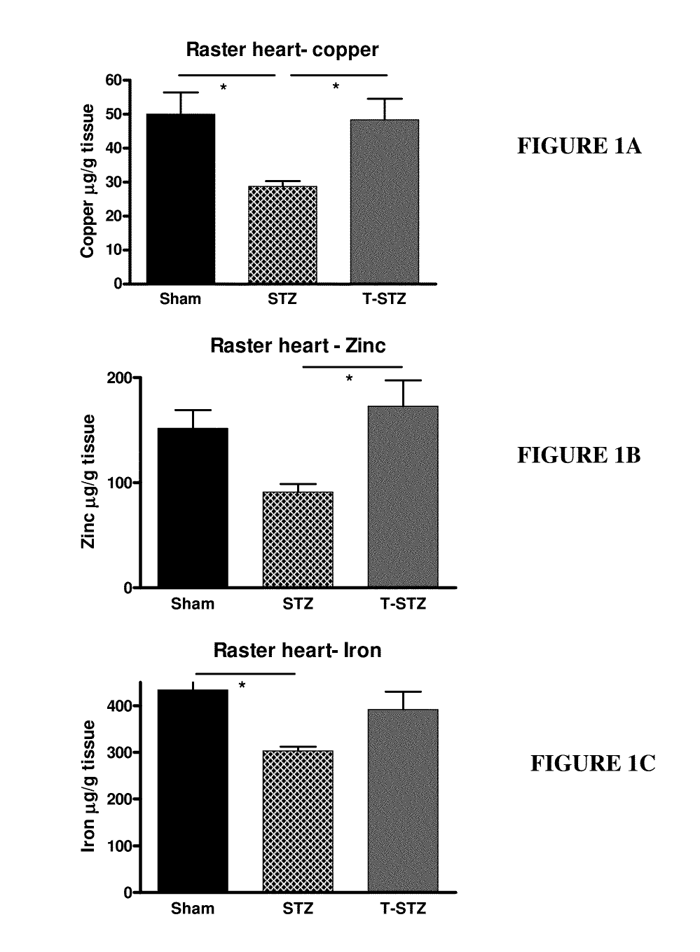

The results showed that there was a statistically significant reduction in total copper levels in STZ rats compared to control rats. Treatment with triethylenetetramine dihydrochloride resulted in a statistically significant increase in total copper, which normalized total copper levels in the T-STZ group to that found in the Sham-control group. See FIG. 1A.

There was also a small, but not statistically significant reduction in the amount of total zinc found in the LV tissue of the STZ rats compared to control rats. Treatment with triethylenetetramine dihydrochloride significantly increased zinc levels, normalizing zinc levels to the levels found within the control group. See FIG. 1B.

While total iron levels were significantly reduced in STZ rats, triethylenetetramine dihydrochloride did not have a statistically significant effect on total iron. See FIG. 1C.

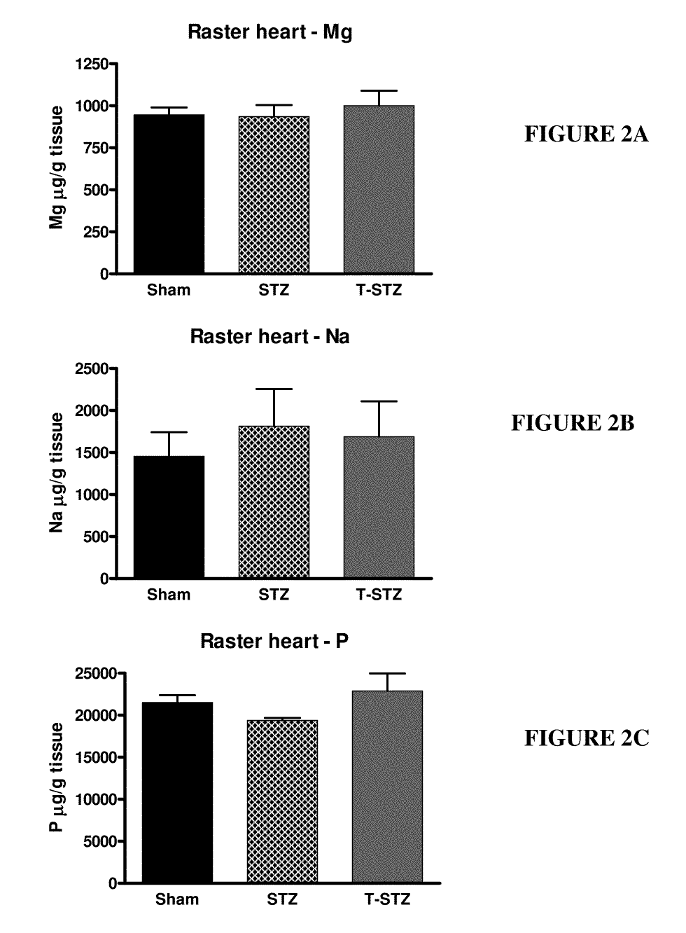

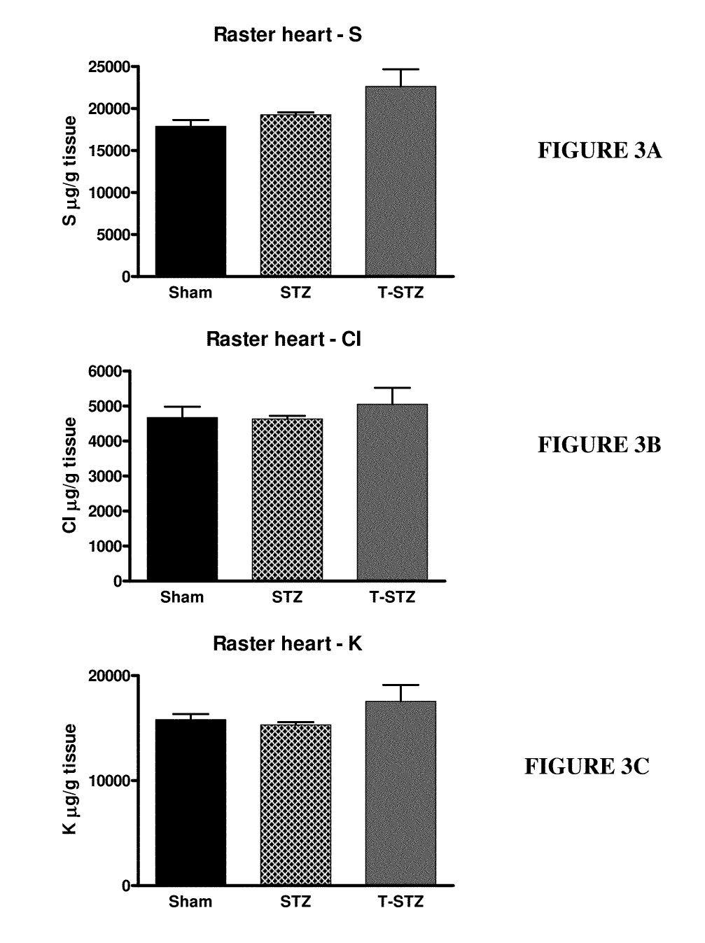

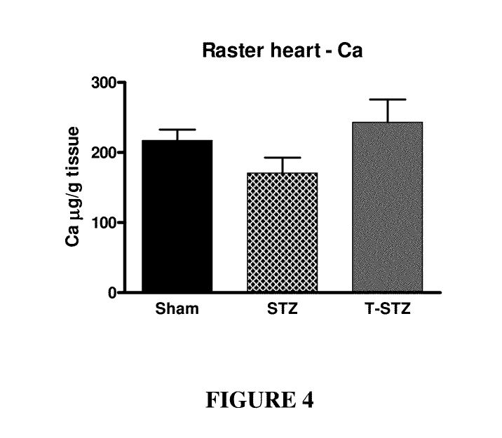

There were no statistically significant differences in levels of total sodium, total magnesium, total calcium, total silicon, total phosphorous, total sulphur, total chloride and total potassium between the STZ, T-STZ and control mice. See FIGS. 2-4.

### Example 2

### Left Ventricle Protein Analysis

Male Wister rats were maintained on Teklad TB 2108 (Harlan UK) rat chow and tap water ab libitum. The rats were randomly assigned to one of three groups: (1) diabetic (STZ); (2) triethylenetetramine di hydrochloride-treated diabetic (T-STZ); and (3) saline treated (control, a/k/a Sham). Rats were made diabetic by injection with 55 mg/kg streptozocin (STZ). Control rats were given a corresponding amount of 0.9% sodium chloride. Blood glucose levels and body weight were measured throughout the 16 weeks using the Advantage II system (Roche Diagnostics). Animals with recurrent glucose levels greater than 11 mM were considered to have established diabetes.

Triethylenetetramine dihydrochloride was administered to the T-STZ group via the drinking water commencing 6 weeks after STZ injections until the end of the trial period (12 weeks). The water intake from the animals was recorded for the intial 6-week diabetes development period. These figures were subsequently used to estimate that a concentration of 50 mg/L in the drinking water was needed to give a drug intake of about 10 mg/day. At the end of the six week treatment period, animals were anesthetized by halothane inhalation, as described in Example 1, and killed. Approximately half of the left ventricle tissue was taken from each animal and cut into 3-4 mm³ sections. These sections were placed into cryogenically stable vials, frozen in liquid nitrogen and stored at −70° C. for proteomic analysis.

The left ventricle tissue was homogenized and the total protein isolated and quantified. Approximately 80-120 mg of left ventricular tissue was diced into approximately 1 mm cubes and weighed. The tissue was homogenised using Ultra Turrax, IKA and 3.5 μl lysis buffer (9 M urea, 8 mM Phenylmethylsulfonyl fluoride (PMSF), 0.1 M Dithiothreitol (DTT), 2% v/v Triton X-100, and 2% v/v Pharmalyte pH 3-10) per 1.0 mg of tissue. Once homogenized, the samples were spun at 13,000 g at 4° C. for 5 minutes to remove cell debris. The supernatant was removed and protein concentration was measured using a 2D Quant kit protein assay (Amersham Biosciences) according to the manufacturer's instructions, except that additional standards were used to provide a more accurate standard curve (0, 5, 10, 15, 20, 25, 30, 35, 40, 45 and 50 μl of BSA).

Isolated proteins were analyzed by two-dimensional electrophoresis. In the first dimension, the proteins were rehydrated into Immobiline DryStrip gels and focused according to their isoelectric points (determined by the net charges of all amino acids in the protein). Briefly, 650 μg of protein was added to 300 μl of rehydration solution (7M Urea, 2M Thiourea, 2% CHAPS, 0.01M DTT, 1% Pharmalyte pH 3-10, and trace Bromophenol Blue) and brought to final volume of 360 μl with milliQ water. These samples were then mixed for 10 minutes at 500 rpm at 25° C. The mixture was then evenly pipetted onto the protean tray and the air bubbles were removed. IPG strips (Immobiline DryStrip gels, 18 cm pH 3-10 NL) were placed gel face down, with the pH 3 end at the anode. The gels were then actively rehydrated at 50V for approximately 24 hours at 20° C.

Directly after active rehydration, isoelectric focusing was performed (Multiphor II Electrophoresis Flatbed Unit, MultiTemp III Thermostatic Circulator and EPS 3500 XL Power supply, all Pharmacia Biotech). The gel bed was cooled to 20° C. The IPG strips were thoroughly rinsed in milliQ water and blotted on their sides and front on damp filter paper. Ondina oil was generously spread onto the flatbed, and the glass drystrip tray was then placed on top. Oil was then poured into the glass dry strip tray and the plastic aligner was placed on top. The IPG strips were then inserted into the grooves of the plastic aligner level to one another, gel-side up, with the pH 3 end towards the top. Damp filter strips were placed across the edges of the IPG strips, and electrodes were placed on top so the gel was in contact with the electrodes. Oil was then poured into the middle, top and bottom compartments, so that the electrodes were sufficiently immersed. Electrophoresis was performed using a gradient voltage as described below:

| Phase | Voltage (V) | Current (mA) | Power (W) | Time (h) |
|---|---|---|---|---|
| 1 | 500 | 1 | 5 | 0.01 |
| 2 | 500 | 1 | 5 | 5 |
| 3 | 3500 | 1 | 5 | 5 |
| 4 | 3500 | 1 | 5 | 15 |

In the second dimension, SDS-PAGE was used to further denature and separate the proteins by their molecular weight. Following isoelectric focusing, the IPG strips were equilibrated for 10 minutes at 21° C. in DTT equilibration buffer (0.05M DTT, 5M Urea, 30% v/v Glycerol, 0.03M SDS, and trace bromophenol blue) with agitation, and subsequently equilibrated with IAA equilibration buffer (0.2M lodoacetoamide (IAA), 5M Urea, 30% v/v Glycerol, 0.03M SDS, and trace bromophenol blue) for 10 minutes at 21° C. with agitation. Ondina oil was spread over the Multiphor flatbed and the pre-cast gel (ExcelGel SDS XL 12-14 gradient gel, Amersham Biosciences) was placed over the oil. The positive buffer strip (ExcelGel SDS Buffer strips, Amersham Biosciences) was placed on the right side of the gel in a straight vertical line, as close as possible to the edge; the negative buffer strip was placed on the left in the same way. The equilibrated IPG strips were blotted on moistened filter paper, and placed next to the negative buffer strip gel-face down. Moist filter paper was put under the ends of the IPG strips, such that half was touching the plastic backing and half touching the gel. Electrodes were positioned over the buffer strips and set down. SDS-PAGE was performed according to the manufacturer's instructions (Amersham Biosciences) at 20° C., with the following parameters (these parameters are for running two gels at the same time):

| Phase | Voltage (V) | Current (mA) | Power (W) | Time (min) |
|---|---|---|---|---|
| 1 | 1000 | 40 | 80 | 45 |
| 2 | 1000 | 80 | 80 | 5 |
| 3 | 1000 | 80 | 80 | 140 |

The IPG strip was removed after phase 1 was complete. At the end of phase 2 the negative buffer strip was place where the IPG strip had been.

Following electrophoresis, gels were fully immersed in fixing solution (10% acetic acid and 50% methanol) for at least 10 minutes with agitation, and then stained with Colloidal Coomassie Blue at 4° C. for at least 24 hours. Afterwards, gels were destained with 1% acetic acid for 1 week and stored at 4° C. in fresh 1% acetic acid to enhance spot detection. Colloidal Coomassie Blue was freshly made prior to staining according to EMBL protocols. Briefly, solution B (1 g Coomassie Blue G-250 dissolved in 20 ml milliQ water) was added to solution A (85 g ammonium sulphate dissolved in 700 ml milliQ water and 18 ml orthophosphoric acid). The solution B container was rinsed with milliQ water twice and added to the mixture until no Coomasie Blue G-250 was left. 170 ml of methanol was then added to the mixture and made up to 1 L with milliQ water.

Gels were digitally scanned and computer analysis of protein expression was performed to determine which proteins were significantly changed (P≦0.05) between the control and untreated diabetic groups, and which proteins were significantly changed in the T-STZ group compared to the STZ group. The computer analysis was performed using ImageMaster 2D Platinum Software version 5.0. These protein comparisons were then statistically analyzed to identify the statistical significance of protein changes.

Gels were digitized by transmittance scanning using an Amersham Biosciences ImageScanner, MagicScan 32 v4.6, and ImageMaster Labscan v3.0. The scanner was calibrated using the Kodak 21 step wedge (R²=0.9902) to convert pixel computer images into densities. Each digitized gel file was then imported into ImageMaster 2D Platinum Software. The image analysis included spot detection, spot editing, reference-gel selection, background subtraction, warping and matching of gels.

Automatic gel warping was performed on all gels, so that marker proteins were matched. The volume of each spot, in each gel was exported for statistical analysis.

The raw data from ImageMaster 2D Platinum Software was imported into JMP statistical software. Mann-Whitney U test was performed between the control and STZ groups, STZ and T-STZ groups, control and T-STZ groups. Each group had 6 gels.

Over 900 protein spots were detected by 2D-electrophoresis. 211 of these proteins were determined to have significantly changed between the control and STZ rats. Of these 211 proteins, 33 were significantly changed by triethylenetetramine dihydrochloride treatment in T-STZ compared to STZ rats. This indicated that treatment with triethylenetetramine dihydrochloride led to normalisation of these proteins. These 33 proteins, along with 2 proteins that were almost significantly (p<0.06) changed by triethylenetetramine dihydrochloride treatment, were considered high interest proteins and were selected for identification by matrix associated laser-desorption ionisation time-of-flight (MALDI-TOF). Several other proteins that were significantly changed between STZ and controls but not significantly affected by triethylenetetramine dihydrochloride treatment were also selected for identification by MALDI-TOF.

These proteins were excised from the gels and stored in 100 mM ammonium bicarbonate solution pH 7.8. A gel blank was included as a negative control. Gel pieces were diced into 1 mm square pieces, and then washed once in milliQ water. Two subsequent washes in 50% acetonitrile with agitation were performed for 15-20 minutes until no Coomassie remained in the gel. Gel pieces were then treated with 60 μl 100% acetonitrile and mixed for 10 minutes, then dried in a speedvac for 10 minutes. Trypsin was added along with extra 100 mM ammonium bicarbonate in an amount sufficient to cover the gel pieces. The samples were incubated overnight at 37° C., with agitation. An equal volume of extraction buffer (50% acetronitrile, 1% TFA and milliQ water to volume) and samples were sonicated in an ice water bath for about 20 minutes. The supernatant was then retained and speed vacuumed for approximately 10 minutes. The resulting protein pellet was then resuspended in 3 μl of extraction buffer.

1 μl of the trypsinised protein sample and 1 μl of matrix (consisting of 10 mg of α-CHC mixed with 1 ml of 60% Acetonitrile, 3% TFA) were mixed. Then, 1.6 μl of each sample/matrix mix was spotted onto a 10x10-well MALDI-TOF plate. For each sample, a calibration mixture (1:1 calibration mixture: matrix) was spotted in the centre. The samples were allowed to air dry for 15 minutes and then analyzed using MALDI-TOF mass spectrometry.

Mass spectra were determined using the Voyager MALDI-TOF mass spectrometer with the following settings in reflector mode. Voltage settings: Accelerating voltage, 20,000V; Grid voltage, 68%; Guide wire voltage, 0.02%; with 100 ns delay time. Spectrum acquisition: shots per spectrum, 100; mass range, 800-4000D; low mass gate, 500D. Laser intensity was varied from 1650-1850, but most commonly set at 1727. Prior to irradiating unknown samples, the machine was manually calibrated with initial error (m/z)<0.01. The subsequently resolved isotopic reference masses were used in the calibration mix: Angiotensin 1, 1296.685300; ACTH (1-17), 2093.086700; ACTH (18-39), 2465.198900; ACTH (7-38), 3657.929400. Samples with low intensity signal (peaks:noise ratio) were reanalyzed using 5 mg a-CHC/ml 60% Acetonitrile, 3% TFA. The best spectrum (large peaks, low noise) from each sample was used for data base searching.

The following properties were taken into account for positive identification: proximity to MW and pl, percentage coverage of protein, accuracy of trypsin digests i.e. number of missed cleavages and peak intensity (from spectra) of the peptide matches. The MS-Fit program was used for peptide mass fingerprinting, using the SwissProt database (http://prospector.ucsf.edu/ucsfhtml4.0u/msfit.htm). Searches were also performed using other databases, such as MASCOT Peptide mass fingerprint (http://www.matrixscience.com/cgi/searchform.pl?FORMVER=2&SEARCH=PMF) and ProFound peptide mapping (http://prowl.rockefeller.edu/profound_bin/WebProFound.exe) to ensure that a good match was obtained.

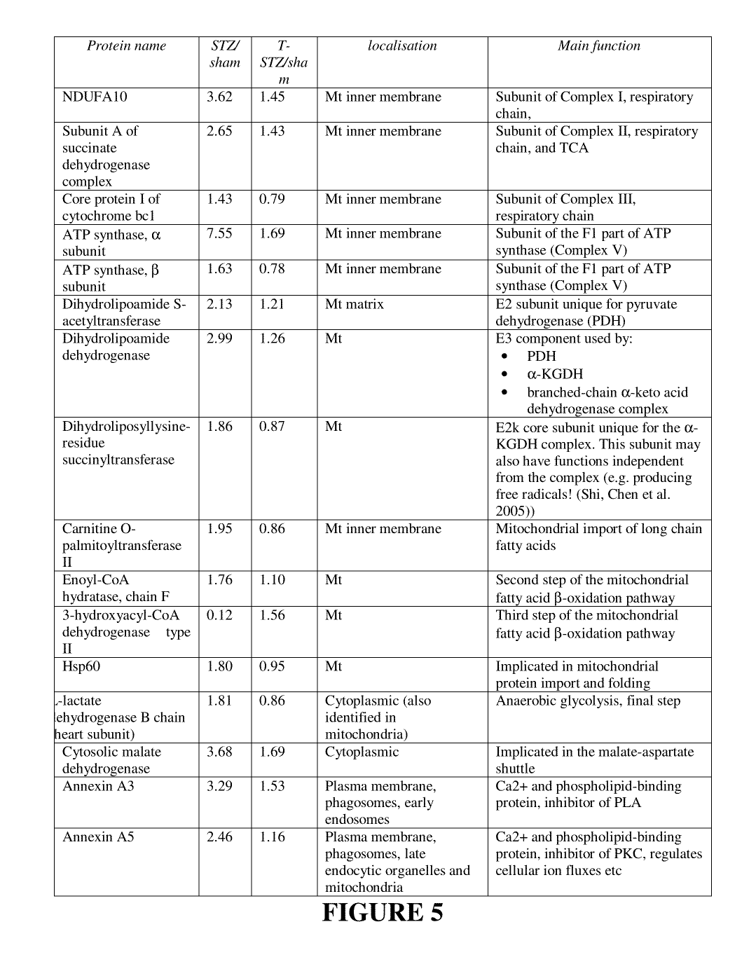

From the group of 211 proteins that were determined to have significantly different expression between the control and STZ groups, 22 have been identified. 14 of these proteins, from the group of 33 proteins, were discovered to be significantly changed back to normal levels in T-STZ rats (p<0.05): NADH dehydrogenase (ubiquinone) 1 alpha subcomplex 10, core protein I of the cytochrome bcl complex, a subunit of ATP synthase, and β subunit of ATP synthase, dihydrolipoamide S-acetyltransferase, dihydrolipoamide dehydrogenase, di hydroliposyllysine-residue succinyltransferase, carnitine O-palmitoyltransferase II, 3-hydroxyacyl-CoA dehydrogenase type II, Heat Shock Protein 60, B chain of L-lactate dehydrogenase, cytosolic malate dehydrogenase, annexin A3, and annexin A5. (FIG. 5). These proteins can be found in the mitochondrial inner membrane, mitochondrial matrix, cytoplasm, plasma membrane, phagosomes, early endosomes, late endocytic organelles and mitochondria.

Two additional proteins were identified that were significantly altered in the STZ group compared to control: chain F of the enoyl-CoA hydratase and subunit A of the succinate dehydrogenase complex. Both these proteins are mitochondrial. These two proteins were not significantly different between T-STZ and control and very close to significantly different between T-STZ and STZ (p<0.06). (See FIG. 5)

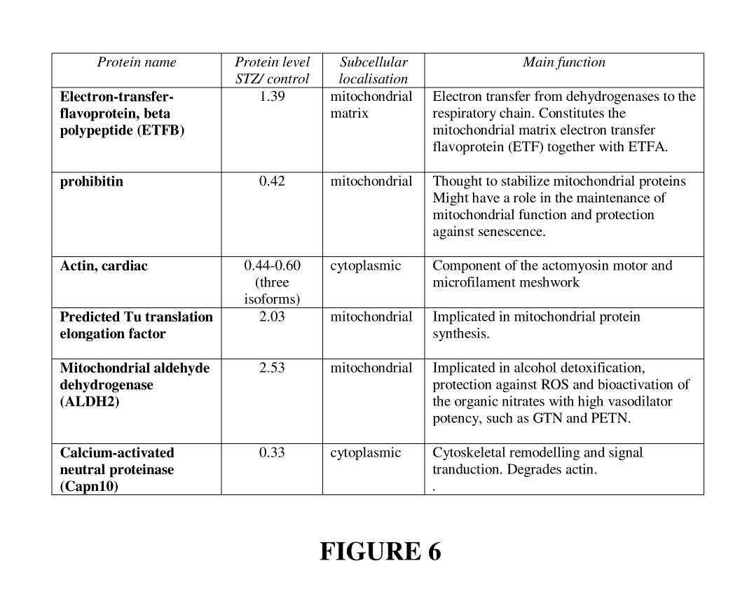

Another six proteins were identified that were significantly altered in STZ rats, but which were not significantly changed by triethylenetetramine dihydrochloride in T-STZ rats: electron-transfer-flavoprotein, beta polypeptide, prohibitin, three isoforms of cardiac actin, and mitochondrial aldehyde dehydrogenase (ALDH2). (See FIG. 6)

### Example 3

### Stabilization of Mitochondria

Male, ZDF rats were maintained on Teklad TB 2108 (Harlan UK) rat chow and tap water ab libitum. Weights and blood glucose levels were monitored periodically throughout the 12 weeks. Animals with recurrent glucose levels greater than 11 mM were considered to have established diabetes. Glucose levels in obese ZDF rats stayed above 11 mM throughout the 12 weeks. Control animals had blood glucose levels between 5-6 mM.

Male, obese ZDF rats (fa/fa, n=4) and their lean littermates (+/?, n=4) were anaesthetized as described in Example 1 and sacrificed via cervical dislocation. The hearts were rapidly removed, and immediately placed into 10 mL of ice-cold isolation buffer (225 mM mannitol, 75 mM sucrose, 20 mM HEPES, 1 mM EGTA and 0.5 mg/mL BSA, at pH 7.4 at 4° C.). The tissue was finely chopped with scissors, incubated with 5 mg protease XXIV, (Sigma, # P38038) for 10 minutes, and then homogenised with an Ultra Turrax homogeniser. The volume was then increased to 30 mL with isolation buffer and centrifuged at 1000 g for five minutes at 4° C. The supernatant was filtered through fine mesh filters and centrifuged again at 7700 g, 4° C. The membranous layer was removed with a soft brush and the supernatant removed. The mitochondrial pellets were resuspended in 30 mL isolation buffer and centrifuged again at 7700 g 4° C. The Mitochondrial pellets from each group were resuspended in 2 mL homogenization buffer and centrifuged again at 7700 g 4° C. The mitochondrial pellets from each group were resuspended in 2 mL homogenization buffer. Mitochondrial protein was determined using the biccichonic acid assay (Peirce Scientific) according to the manufacture's instructions.

Mitochondrial swelling (stability) assays were adapted from Lapidus and Sokolove. Briefly, mitochondria were resuspended at a concentration of 0.2 mg.mL¹ in 200 mM sucrose, 10 mM MOPS, 5 mM succinate, 1 mM Pᵢ, 10 μM EGTA, 2 μM rotenone at pH 7.4 and incubated at 30° C. for 10 minutes. Absorbance was then followed at 540 nm using a Molecular Devices Spectramax Plus plate reader for 30 minutes to determine the background swelling. In all experiments, 750 M ADP was added.

Four mitochondrial swelling assays were conducted. The first set tested mitochondrial stability following incubation with a range of spermine, spermidine, and triethylenetetramine dihydrochloride concentrations (0-5 mM). A second experiment repeated the first but with the addition of CaCl₂. The third experiment involved incubation of mitochondria in a high background concentration of spermine (5 mM) with a range of triethylenetetramine dihydrochloride concentrations (0-5 mM). The fourth experiment repeated experiment three but with the addition of CaCl₂.

In the first experiment suspended mitochondria were incubated in ADP to 750 mM plus spermine, spermidine or triethylenetetramine dihydrochloride to final concentrations of 5, 2.5, 1.25, 0.613, 0.312, 0.156, 0.078 and 0 mM. The absorbance at 540 nm was then followed for 30 minutes. As a decrease in absorbance represents mitochondrial swelling, a decrease in area under the time/absorbance curve represents swelling of mitochondria over the 30 minutes. Therefore an increase in area under the curve represents shrinkage of mitochondria.

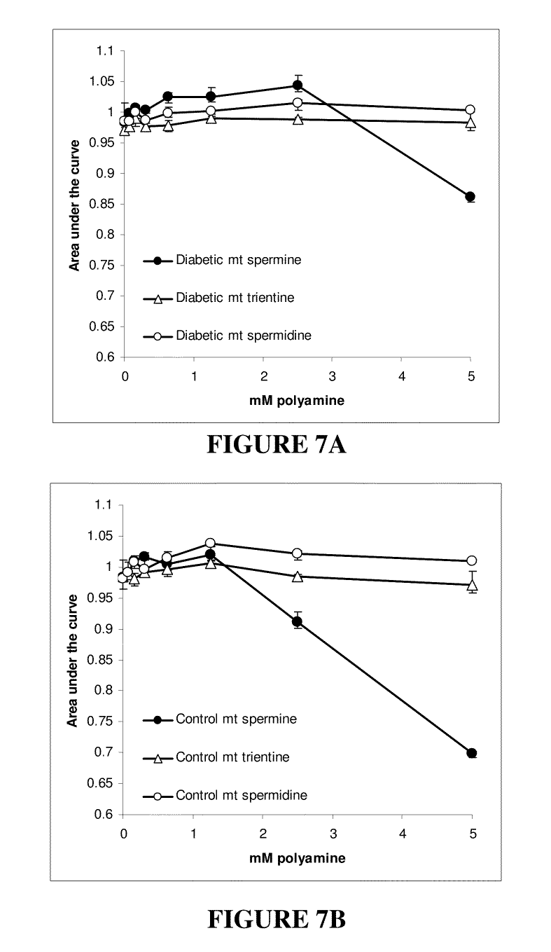

There was no detectable change in mitochondrial volume in diabetic or control mitochondria exposed to spermidine or of triethylenetetramine di hydrochloride. Addition of spermine had similar effects as spermidine and trientine at lower concentrations but induced swelling at concentrations above 2.5 mM in mitochondria from obese rats and above 1.25 mM in mitochondria from lean rats. See FIGS. 7A and 7B.

The procedure described above was then repeated in a second experiment, but with the addition of 150 μM CaCl₂ to each treatment group. The absorbance at 540 nm was followed and the area under the curve was then calculated over the 30 minute period.

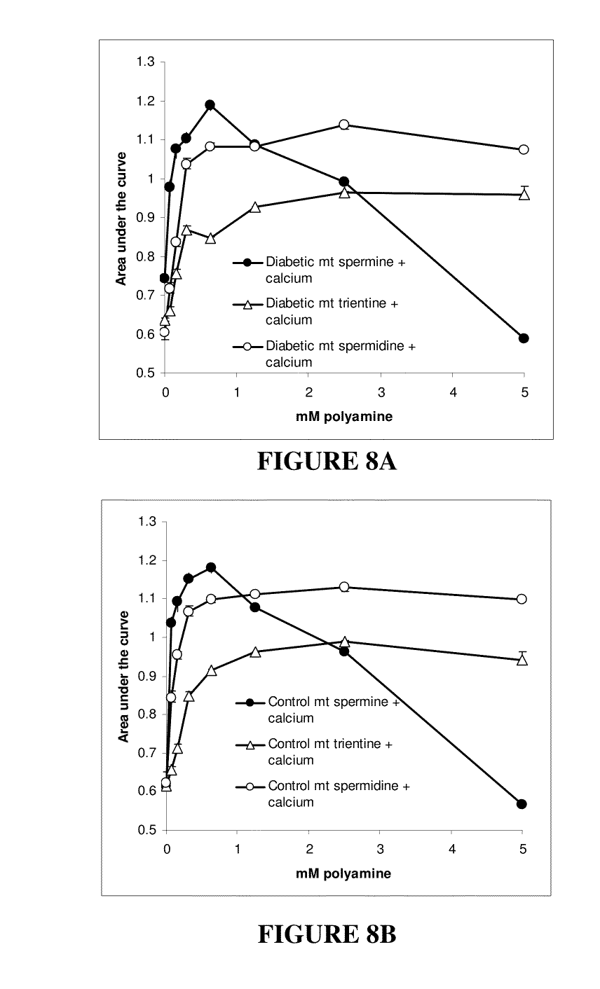

Both diabetic and control mitochondria swell with the addition of 150 μM Ca²⁺ (data not shown). Spermine, spermidine and triethylenetetramine dihydrochloride all inhibit swelling at concentrations below 0.625 mM, with spermine providing the greatest inhibition of swelling. However above 0.625 mM spermine appears to induce swelling, while spermidine and triethylenetetramine continue to protect mitochondria from swelling in mitochondria from both obese and lean rats. See FIGS. 8A and 8B.

A third experiment using the same procedure above with isolated mitochondria were incubated with 5 mM spermine and 750 μM ADP, and then exposed to various amounts of triethylenetetramine dihydrochloride (5, 2.5, 1.25, 0.613, 0.312, 0.156, 0.078 and 0 mM) and the absorbance at 540 nm was then followed for 30 minutes.

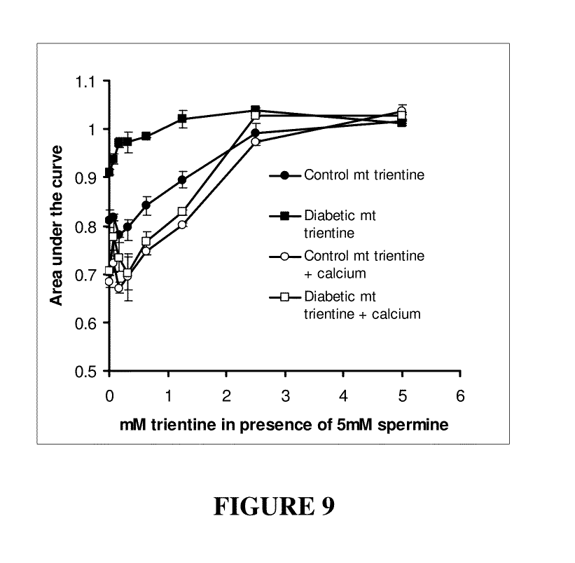

In a fourth experiment, this procedure was repeated with the addition of 150 μM CaCl₂. The area under the curve was then calculated over a 30 minute period. Triethylenetetramine dihydrochloride inhibits spermine induced swelling at lower concentrations for diabetic mitochondria than for control mitochondria. With the addition of 150 M Ca²⁺ this difference is lost, demonstrating spermine and Ca²⁺ induced swelling are independent. Triethylenetetramine di hydrochloride reduces Ca²⁺ induced mitochondrial swelling equally well in in diabetic and control mitochondria at and this protective effect was shown to be variable by concentration. See FIG. 9.

### Example 4

### Left Ventricle mRNA Expression

Wistar rats (starting body weight between about 220-250 g) were maintained on Teklad TB 2108 (Harlan UK) rat chow and tap water ab libitum. The rats were randomly assigned to two groups: (1) diabetic (STZ) or (2) saline treated (control). The rats in the STZ group were injected with 60 mg/kg streptozocin (STZ), while the rats in the control group were given a corresponding amount of 0.9% sodium chloride. Blood glucose levels and body weight were measured prior to injection, two days after injection, and weekly thereafter. Animals with sustained glucose levels greater than 11 mM were considered to have established diabetes.

At the beginning of week seventeen, animals were anesthetized by halthane inhalation (5% halothane and 2 L/min oxygen) as described in Example 1 and killed by cervical dislocation.

Rat hearts were excised in an RNase enzyme free environment. Briefly, the chest was cut open and any connective tissue was cut from the heart. The heart was handled using sterile blunt nosed forceps to reduce damage to the tissue. The aortic remnant of the rat heart was ligated to the metal cannula to allow perfusion using a GENIE 220 infusion pump with 40 mL (STZ) or 60 ml (Control) 1×PBS 4° C. at a flow rate of 15 ml/min. Once perfusion had ended, the left ventricle was cut away from the rest of the heart and placed in a tube containing RNAlater (Qiagen, Germany) and stored at −80° C.

Total RNA from the LV of 28 animals was obtained using either the Qiagen MIDI RNeasy RNA extraction kit or the Ambion Mini RNAqueaous RNA extraction kit, according to the manufacturer's instructions. The total cell RNA was quantified using the NanoDrop® ND-1000 UV-Vis Spectrophotometer (NanoDrop Technologies, Rockland Del., USA). RNA integrity was determined using the Agilent Bioanalyzer. RNA with an RNA integrity number (RIN) of 8.5 or above was deemed to be of a high enough quality for use on the Affymetrix Microarray platform.

RNA expression levels were measured via the Affymetrix GeneChip system according to the manufacturer's instructions. cRNA was synthesized from the RNA as per the protocol provided with the Affymetrix GeneChip system. The resultant cRNA was hybridised to the microarray chip (Affymetrix Rat GeneChip 230 2.0) overnight before the excess was washed off and a fluorescent label was attached for visualization of cRNA bound to the probe sets. GeneChips were scanned using Affymetrix GeneChip Scanner 3000 and processed using GCOS (Affymetix). This data was then analyzed using a number of statistical methods to identify any differences in levels of RNA between the diabetic and normal animals.

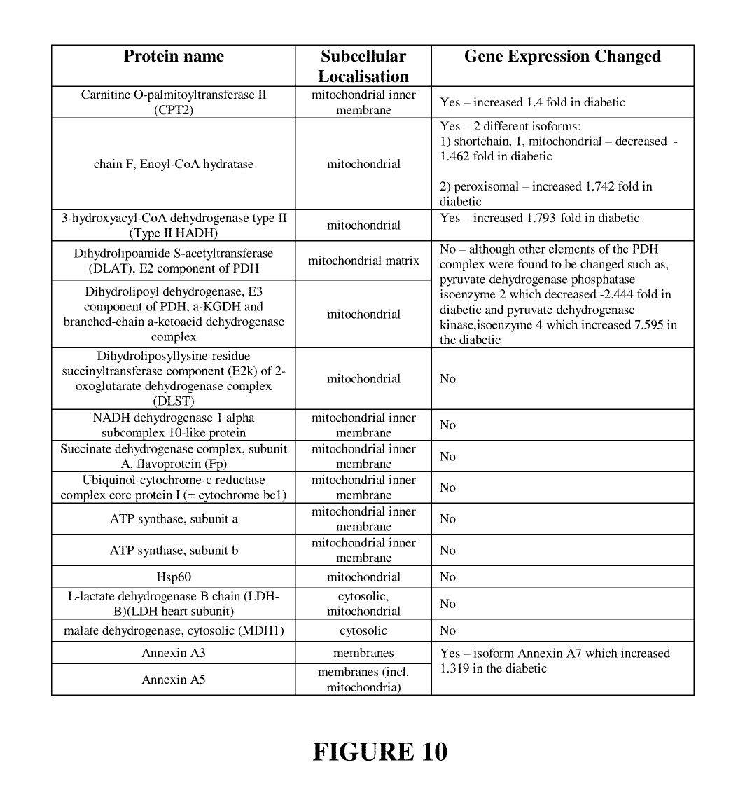

Between the STZ and control groups, over 900 gene changes occurred which were found to be significant based on P-value and LogOdds scores. Analysis of these 900 genes found that only 321 of them were annotated in the literature enough to give a sufficient description of their function. Of these 321 genes 71 have been associated with the mitochondria (approximately 20%). mRNA expression for the 16 proteins identified in Example 2 were specifically analyzed and described in FIG. 10. Carnitine O-palmitoyltransferase II had a 1.4 fold increase in expression in diabetic animals. Chain F of the enoyl-CoA hydratase had a 1.7 fold increase in the peroxisomal isoform in diabetic animals. 3-hydroxyacyl-CoA dehydrogenase type II was increased by 1.8 fold in diabetic animals and annexin A7 was increased by 1.3 fold in diabetic animals. See FIG. 10.

### Example 5

### Normalization of EC-SOD, TGF-β1, Smad 4, and Collagen IV RNA Expression

Male Wister rats were maintained on Teklad TB 2108 (Harlan UK) rat chow and tap water ab libitum. The rats were randomly assigned to one of three groups: (1) diabetic (STZ); (2) triethylenetetramine di hydrochloride-treated diabetic (T-STZ); and (3) saline treated (control). Rats were made diabetic by injection with 55 mg/kg streptozocin (STZ). Control rats were given a corresponding amount of 0.9% sodium chloride. Diabetes was confirmed by blood glucose measurement 24 hours after STZ injection. Animals with glucose levels greater than 11 mM were diagnosed with diabetes. The body weight and blood glucose were monitored weekly for 16 weeks using the Advantage II system (Roche Diagnostics).

T-STZ rats were administered triethylenetetramine dihydrochloride via the drinking water at a dose of 20 mg/day. Treatment began at the beginning of week 9 after STZ injection and continued for eight weeks. At the end of the eight-week treatment, animals were anesthetized by halthane inhalation (5% halothane and 2 L/min oxygen).

The rat hearts and aortas were excised in an RNase enzyme free environment using sterile, blunt nosed forceps to reduce damage to the tissue. The aorta and heart were perfused or washed free of blood in DEPC-treated phosphate-buffered saline (PBS, pH7.4). These tissues were then stored in RNAlater (Ambion) overnight at 4° C. before storage at −80° C. for RNA isolation.

RNA from the aorta and LV was obtained using the Qiagen MIDI RNeasy RNA extraction kit, according to the manufacturer's instructions. Briefly, approximately 100 mg of each tissue was sliced, and homogenized with an electrical homogenizer in 3 ml lysis buffer. The RNA concentration was measured spectrophotometrically using a Narodrop, and the RNA integrity was checked by agarose gel electrophoresis. 1 μg of total RNA was treated with RQ1 RNase free DNase (Promega, Madison, Wis.) at 37° C. for 30 min, and was reverse-transcribed with random hexamers and SuperScript™ III Reverse Transcriptase (Invitrogen).

mRNA expression levels were compared by quantitative real-time PCR analysis with ABI Prism 7900 HT Sequence Detection System (Applied Biosystems, Foster City, Calif.). ROX was used as a passive reference in each sample to normalize for non-PCR related fluctuations in fluorescence signal. Reactions were prepared in the presence of the fluorescent dye SYBR green (Applied Biosystems) for specific detection of double-stranded DNA. The cDNA amount used in the PCR was 0.25 ng for 18S, 1.0 ng for TGF-β1 and Smad 4 or 1.5 ng for EC-SOD and Collagen IV. Primer concentrations used were 0.1 μM for 18S, and 0.4 μM for EC-SOD, collagen IV, Smad 4 and TGF-β1. The PCR conditions used for TGF-β1, Smad 4 and EC-SOD were 95° C. for 10 minutes, followed by 40 cycles of 95° C. for 15 seconds then 58° C. for TGF-61, 60° C. for 18S and Smad 4 or 61° C. for EC-SOD for 1 min. PCR conditions for collagen IV was 95° C. for 10 min, followed by 50 cycles of 95° C. for 15 s, 55° C. for 30 s and 72° C. for 30 s. Primers used in PCR amplification include:

(a) EC-SOD
(b) Forward 5′ to 3′:
(SEQ ID NO: 1)
GGCCCAGCTCCAGACTTGA
(c) Reverse 5′ to 3′:
(SEQ ID NO: 2)
CTCAGGTCCCCGAACTCATG
(d) TGF-β1:
(e) Forward 5′ to 3′:
(SEQ ID NO: 3)
TTCCTGGCGTTACCTTGGT
(f) Reverse 5′ to 3′:
(SEQ ID NO: 4)
GCCACTGCCGGACAACT
(g) Collagen IV:
(h) Forward 5′ to 3′:
(SEQ ID NO: 5)
GAAAACCTATTCCATCGACTGTGA
(i) Reverse 5′ to 3′:
(SEQ ID NO: 6)
ACCTGACAGCGGCTTATGATTT
(j) Smad 4:
(k) Forward 5′ to 3′:
(SEQ ID NO: 7)
AGTCAGCCGGCCAGCAT
(l) Reverse 5′ to 3′:
(SEQ ID NO: 8)
GAAGCTATCTGCAACAGTCCTTCAC

After PCR amplification, dissociation curves were constructed and PCR products were subjected to agarose gel electrophoresis to confirm formation of the specific PCR products. The levels of gene expression of the target sequences were normalized to that of the active endogenous control, 18 s, to control for variations in the amount of DNA available for PCR in the different samples. Relative quantitation of mRNA expression was performed as described in User Bulletin #2 (Applied Biosystems) using the standard curve prepared from serially diluted cDNA samples.

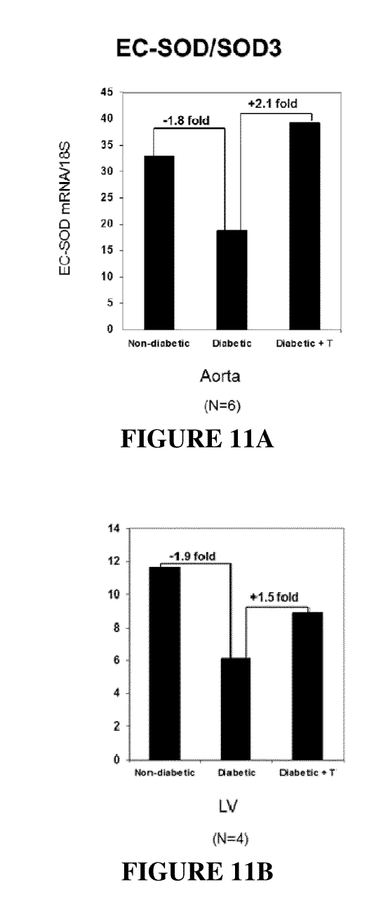

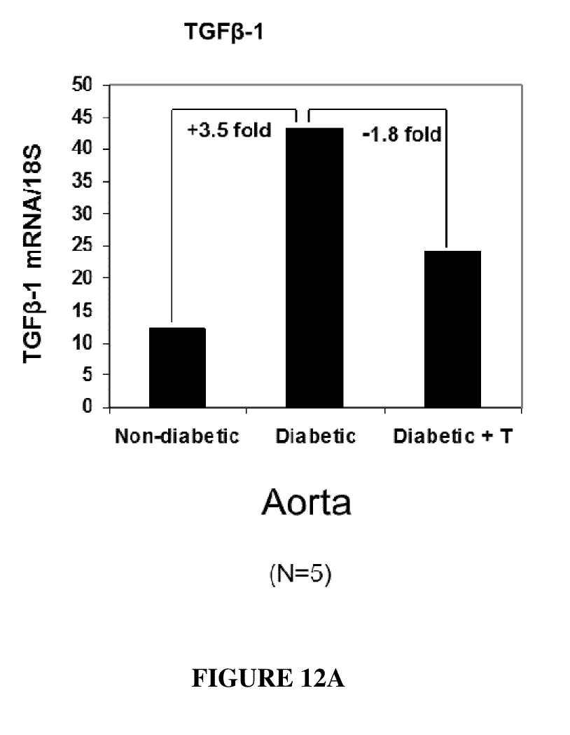

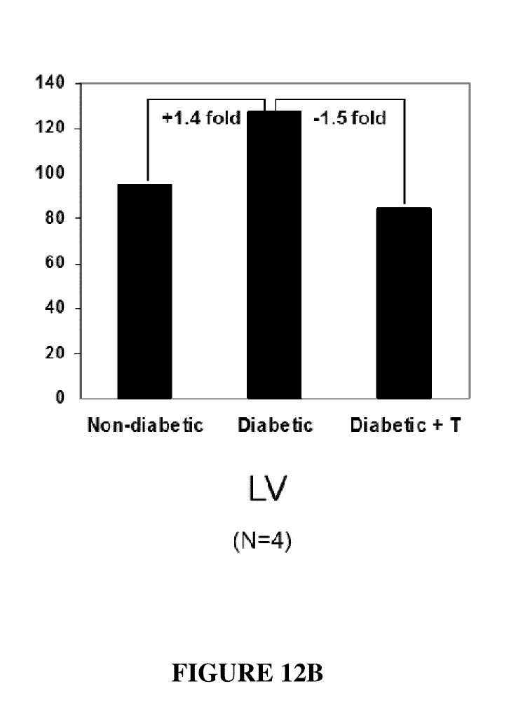

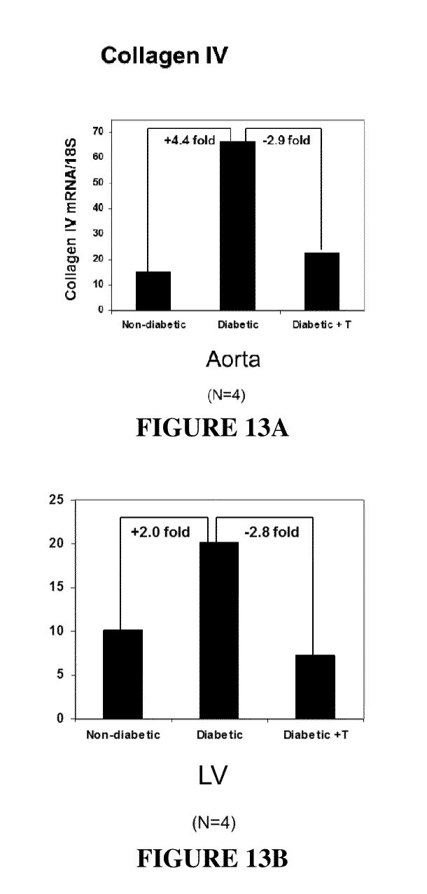

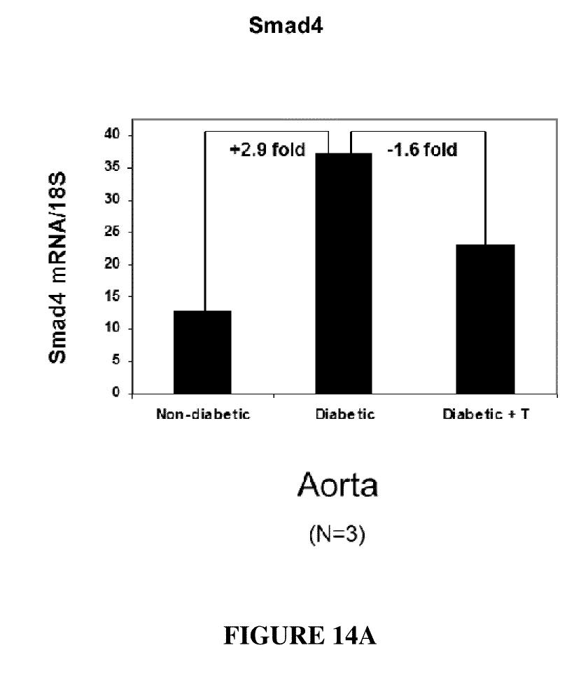

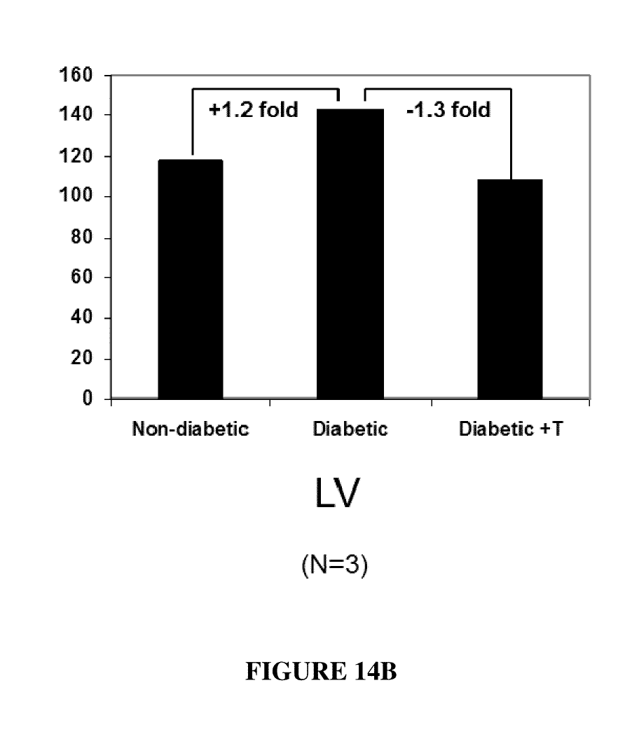

These results show that EC-SOD mRNA expression in STZ animals was decreased by 1.8 fold in the aorta and 1.9 fold in the left ventricle and that mRNA levels were normalized in T-STZ animals. (See FIGS. 11A and 11 B) TGF-61 mRNA expression levels were significantly up-regulated in STZ animals. This up-regulation was normalized with triethylenetetramine dihydrochloride treatment. (See FIGS. 12A and 12B) Collagen IV mRNA levels were increased in the aorta and LV of STZ rats. These levels were normalized in T-STZ rats. (See FIGS. 13A and 13B). Additionally, Smad 4 mRNA levels were increased in STZ animals and normalized in T-STZ animals. (See FIGS. 14A and 14B).

### Example 6

### Effects of Polyamines on Cytochrome C Release

We first examined the effects of spermine, spermidine and triethylenetetramine di hydrochloride on cytochrome c release. Mitochondria were isolated according to the methods described in Example 3. The isolated mitochondria was added to a final concentration of 0.2 mg/ml in swelling buffer supplemented with 0.75 mM ADP and varying concentrations either: (1) spermine, (2) spermidine or triethylenetetramine dihydrochloride. Following incubation at 37° C. for 0, 30, 60 and 90 minutes, mitochondria were pelleted by centrifugation at 12,000×g for 5 minutes. Supernatants were aspirated and both pellets and supernatants were stored at −20° C. until analysed. Western blotting was used to anaylize levels of cytochrome c released from the mitochondrial intermembraneous space using standard western blot protocols and a specific antibody for cytochrome c (monoclonal mouse-anti-cytochrome c, clone 7H8.2C12 from Becton Dickinson Ltd.).

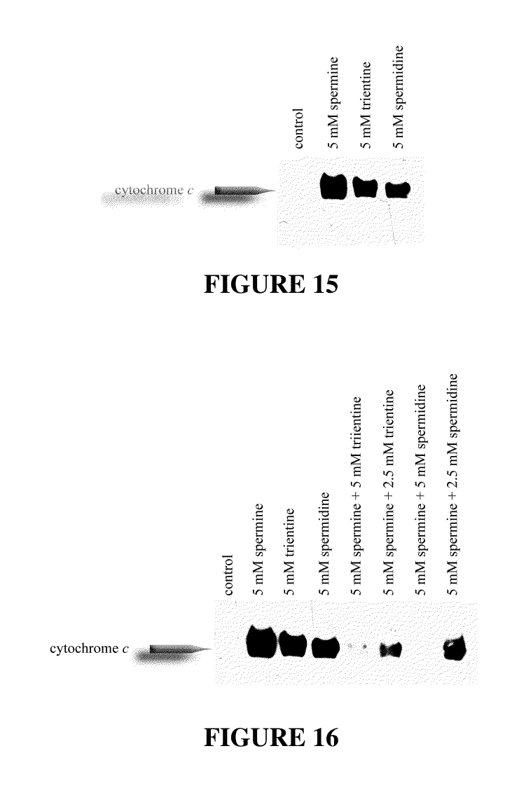

Western blotting showed that maximum release of cytochrome c was obtained after 30 min of incubation with spermine (data not shown). Mitochondria incubated (briefly) in the absence of spermine, spermidine or triethylenetetramine dihydrochloride released no detectable cytochrome c to the supernatants. 30 min incubation with 5 mM spermine led to release of large amounts of cytochrome c into the supernatant. Spermidine and trientine also caused cytochrome c release at 5 mM, albeit less than in the case of spermine (See FIG. 15).

Next, we studied the effect of co-incubating the mitochondria with 5 mM spermine and varying concentrations of spermidine or trientine. The mitochonria obtained as described above was incubated with either (1) 5 mM spermine, (2) 5 mM trietheylentetramine dihydrochloride, (3) 5 mM spermadine, (4) 5 mM spermine+5 mM triethylenetetramine dihydrochloride, (5) 5 mM spermine+2.5 mM triethylenetetramine dihydrochloride, (6) 5 mM spermine+5 Mm spermidine or (7) 5 mM spermine+2.5 Mm spermidine.

5 mM spermine with either triethylenetetramine dihydrochloride or spermidine led to diminished cytochrome c release from the mitochondria (See FIG. 16) with triethylenetetramine dihydrochloride being more potent than spermidine. This effect was also concentration dependent since co-incubation with 2.5 mM spermidine and trientine were both less potent than when 5 mM was used of the respective polyamine (See FIG. 16).

An enzyme assay was then used to evaluate the mitochondrial pellets to determine the contents of the matrix protein citrate synthase in order to evaluate the integrity of the mitochondria after the respective incubations. Citrate synthase activity was determined according to the methods decscribed in Newsholme and Crabtree, J. Exp. Zoo. 239(2): 159-67 (1986). In brief, frozen mitochondrial pellets were resuspended in a reaction mixture containing 50 mM Tris-HCl (pH8.0), 0.1 mM acetyl coenzyme A and 0.2 mM 5,5′-dithiobis-(2-nitrobenzoic acid) (DTNB). Reactants were incubated for five minutes prior to measurement and the assay started by the addition of 5 mM oxaloacetate. Absorbance of DTNB was then followed at 412 nm and units were calculated relative to soluble protein using the biccichonic acid method (BCA, Pierce) with bovine serum albumin as standard.

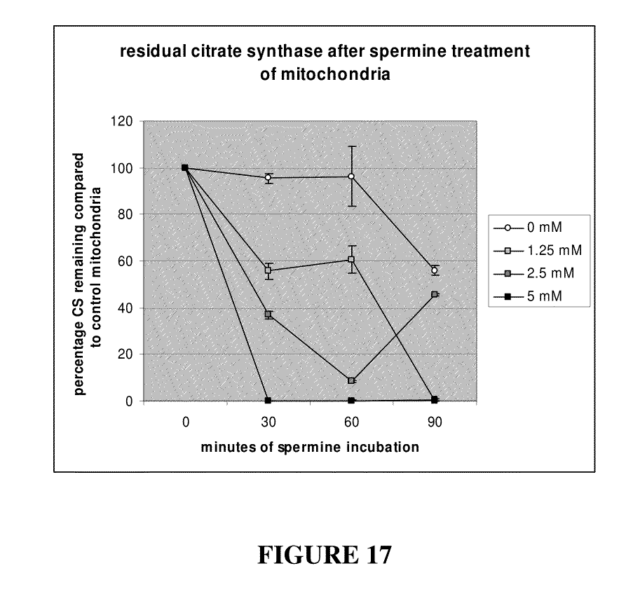

Citrate synthase activity was used as a marker for the integrity of the inner mitochondria membrane. Enzyme assays of citrate synthase in the mitochondrial pellets showed that the activity in the absence of polyamine addition was stable over 60 min of incubation but lower after 90 min, indicating that mitochondrial integrity was maintained for at least 60 min (FIG. 17). However, treatment with 5 mM spermine led to a rapid loss of citrate synthase activity, evidence that this concentration of spermine led to a rupture of the mitochondrial membranes (FIG. 17). This effect appeared to be concentration dependent; lower concentrations of spermine had less severe impact on the residual citrate synthase activity (FIG. 17).

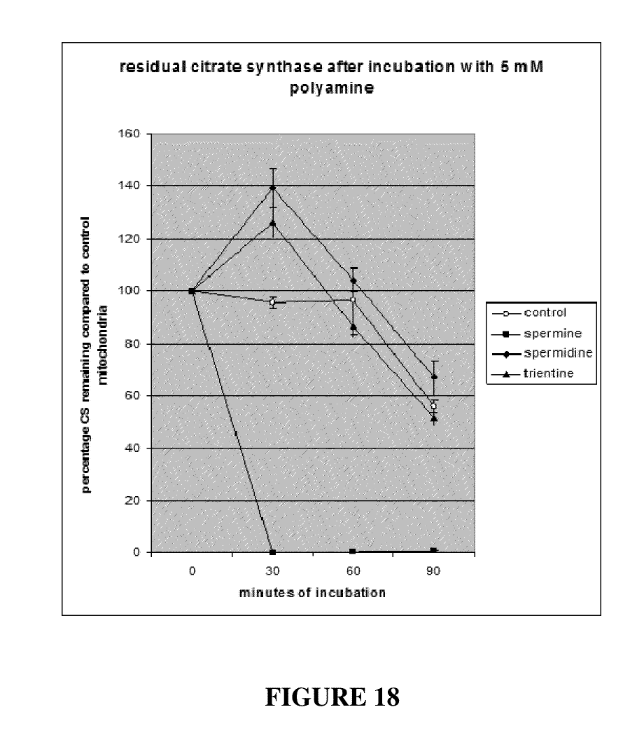

The loss of residual citrate synthase activity associated with spermine incubation indicated that cytochrome c release observed in response to 5 mM spermine (FIGS. 15 and 16) was not selective for cytochrome c. Instead, it may be due to disruption of the mitochondrial membranes, leading to general leakage of mitochondrial proteins. In contrast, incubation with 5 mM spermidine or triethylenetetramine dihydrochloride led to increased amounts of residual citrate synthase activity compared to control samples after 30 min and similar levels as controls at later time points (FIG. 18). This demonstrates that cytochrome c release in response to incubation with triethylenetetramine dihydrochloride or spermidine (as showed in FIG. 16) may be selective, in contrast to that of spermine.

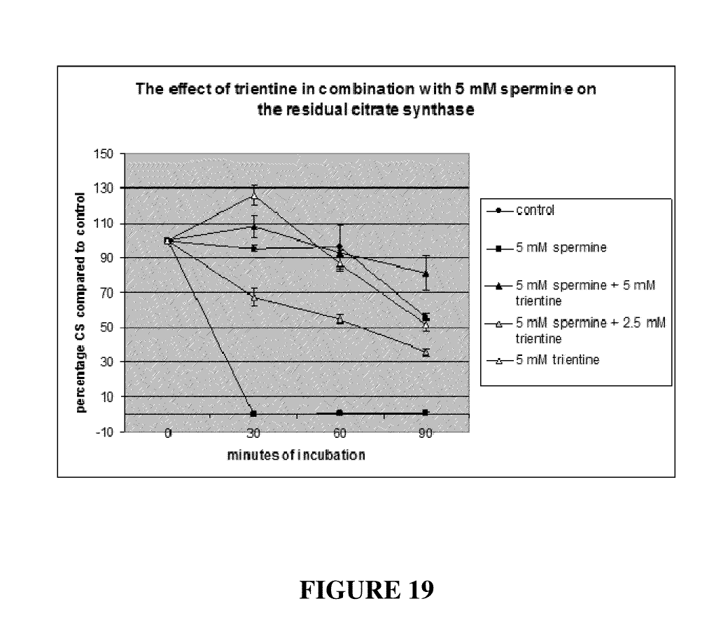

Co-incubation with 5 mM triethylenetetramine dihydrochloride and 5 mM spermine led to an almost complete retention of residual citrate synthase over 60 min (FIG. 19), with values very similar to those of both controls and 5 mM triethylenetetramine dihydrochloride only. At 90 min of incubation, both control mitochondria and mitochondria incubated with 5 mM triethylenetetramine dihydrochloride had lost almost half of their citrate synthase activity whereas mitochondria coincubated with 5 mM spermine and 5 mM triethylenetetramine dihydrochloride retained 80% of the citrate synthase activity at this point. (See FIG. 19).

Co-incubation with 5 mM spermine and a lower triethylenetetramine dihydrochloride concentration (2.5 mM) resulted in a faster degradation of citrate synthase compared to the higher concentration of triethylenetetramine dihydrochloride, however this concentration still protected against the rapid and total loss of citrate synthase activity seen in mitochondria incubated with 5 mM spermine alone. This revleals that the protective effect of triethylenetetramine dihydrochloride may be concentration dependent. (See FIG. 19)

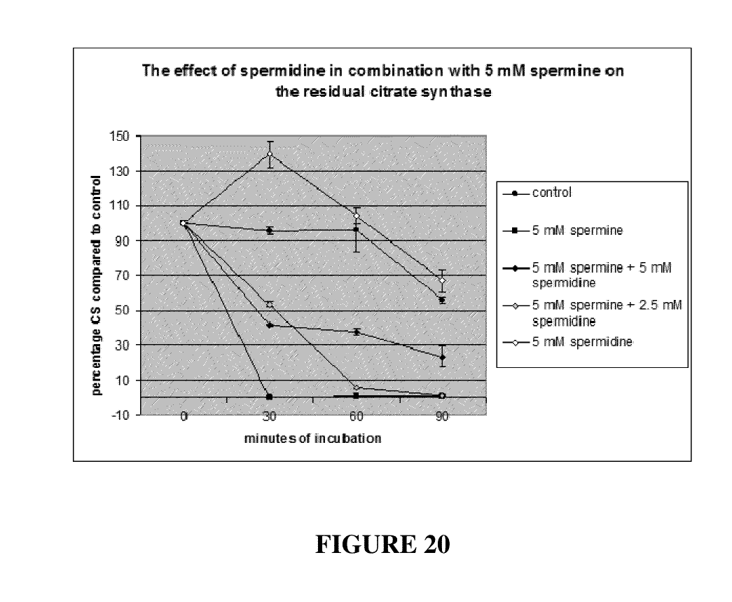

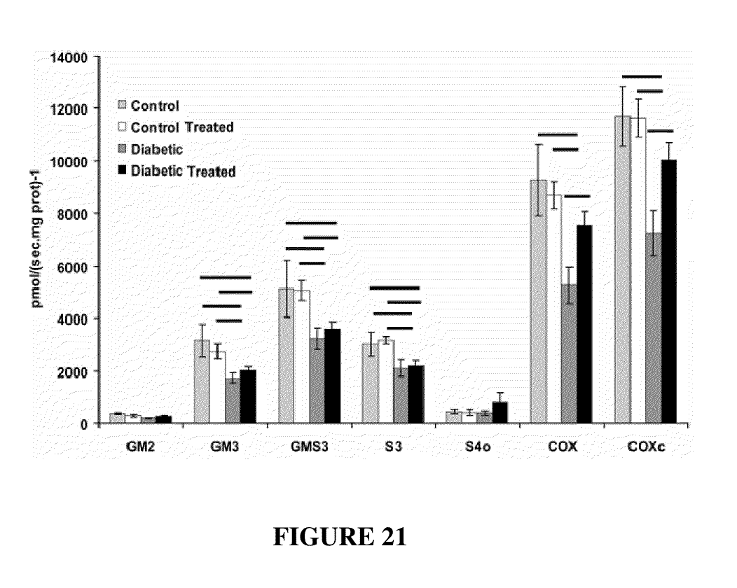

Spermidine was also capable of improving residual citrate synthase activity in the presence of 5 mM spermine although it was markedly less potent than triethylenetetramine dihydrochloride (FIG. 20). These results also reveal a concentration dependency.

### Example 7

### Effects of Polyamines on Left Ventricle Mitochondria of Diabetic Animals

Male Wistar rats (starting body weight from about 200 to about 250 g) were maintained on Teklad TB 2108 (Harlan UK) rat chow and tap water ab libitum. At eight weeks the rats were randomized into two groups: control and diabetic (STZ). Rats were made diabetic by injection with 60 mg/kg STZ and the control rats were given a corresponding amount of 0.9% sodium chloride. Rats injected with STZ became diabetic within two days as determined by a blood glucose level >11 mM. Once diabetes was established, these two groups were then randomized into a further two groups (four groups in total); (1) diabetic treated with triethylenetetramine disuccinate; (2) untreated diabetic; (3) control treated with triethylenetetramine disuccinate; and (4) untreated control.

Triethylenetetramine disuccinate was dissolved into Milli-Q water and administered as the drinking water at a rate of 30 mg/day for 11 weeks (total trial period was 19 weeks). The control groups received Milli-Q water ab libitum during the corresponding period. At the end of the eleven week treatment period, rats were anaesthetized with isoflurane, the abdominal cavity was opened and a catheter inserted into the vena cava. Approximately 1 mL of blood was removed for future analysis and 10000 U/Kg heparin infused. After two minutes the thoracic cavity was opened and the heart excised and placed into ice-cold mitochondrial isolation buffer and perfused retrograde with ice-cold 50 mL mitochondrial isolation buffer. The mitochondrial extraction buffer (buffer A) consisted of: −10 mM HEPES pH 7.5 (at 4° C.), 200 mM mannitol, 70 mM sucrose and 1 mM EGTA.

The heart was then blotted dry, weighed, and transected midway dorso-ventrally in order to measure the left ventricle, septum and right ventricular walls using electronic micrometer callipers (results not shown). The left ventricle was opened by a cut to the septum and fibres removed from the opposing endomyocardium and placed into BIOPS media, (a relaxing solution), containing 2.77 mM CaK₂EGTA, 7.23 mM K₂EGTA (free Caₜ concentration 0.1 μM), 20 mM imidazole, 20 mM taurine, 6.56 mM MgCl₂, 5.77 mM ATP, 15 mM phosphocreatine, 0.5 mM dithiothreitol, and 50 mM K-MES, pH 7.1. Myocardial fibers were permeabilised by agitation for 30 min at 4° C. in the relaxing solution supplemented with 50 μg/ml saponin. Fibers were washed in ice-cold respiration medium by agitation for 10 min.

Fiber respiration was then measured in a respirometer at 30° C. at high resolution using Clark-type electrodes and integrated software that was used for data acquisition and analysis (DatLab 4, Oroboros, Oxygraph; Innsbruck, Austria). The respiration medium consisted of: −110 mM sucrose, 60 mM K-lactobionate, 0.5 mM EGTA, 1 g/l BSA essentially fatty acid free, 3 mM MgCl2, 20 mM taurine, 10 mM KH2PO4, 20 mM K-HEPES, with the pH at 7.1. The O₂ solubility of this medium was taken as 10.5 μM/kPa. Respiratory rates (oxygen fluxes) were expressed as pmol O₂. (sec.per milligram of tissue wet weight)⁻¹.

The following titration respiration assay was carried out in the respirometer to measure the function of the electron transport chain (ETC) components, specifically complexes I, I & II, II and IV and the phosphorylation capacity of complex V by titration with multiple substrates and inhibitors. The following substrates were used to measure the flux rates through the various complexes:—glutamate 10 mM, malate 5 mM, ADP 1.25 mM, succinate 10 mM, rotenone 0.005 mM, oligomycin (2 ug/mL), FCCP 0.0005 mM, antimycin 0.0025 mM, TMPD 0.5 mM and ascorbate 2 mM. The intactness of the outer mitochondrial wall was tested by the addition of cytochrome C (0.1 uM). Glutamate and malate provide a measure of flux through complex I, succinate through complex II, glutamate, malate and succinate through complexes I and II and provide an indication of complex III (at the “Q-junction”). FCCP is an uncoupling agent which can also be used to estimate maximal flux rates without phosphorylation. TMPD and ascorbate provide an indication of flux through complex IV (COX) and the addition of cytochrome C is informative of outer mitochondrial membrane damage.

Respiratory flux rates through complex I (GM2 and GM3), complexes I and II (GMS3) and II (S3) were determined to measure the intactness of the individual complexes (GM3 and S3), and to estimate maximal flux rates through the electron transport chain. The flux rate through both complexes I and II combined (GMS3) was measured to also determine if flux rates were additive and therefore provide some insight to flux through complex III. Estimates of proton leak rates were made by measurement of state 2 and 4 respiration by measurement of flux prior to addition of ADP (GM2) and following addition of succinate)(S4^{o}). Re-oxygenation was performed when oxygen saturation approached 50% to ensure oxygen was not rate limiting. GM2—is the respiration flux through complex I in the absence of ADP and uncoupling agents (FCCP, dinitrophenol), which provides an indirect measure of the proton leak rate through the inner mitochondrial membrane (state 2 respiration). Flux rates determined following the addition of glutamate and malate and ADP (GM3) provides a measure of flux through complex I with phosphorylation (i.e. the phosphorylation of ADP to ATP, state-3 respiration). GMS3 provides a measure of state-3 flux through complexes I and II following respiration on glutamate, succinate following inhibition of complex I with rotenone, and S4^{o} provides a measure of respiratory flux with complex V blocked by oligomycin (non-phosphorylating, similar to GM2). S4^{o} provides another measure of proton leak rate (4 refers to state 4 respiration where the superscript ^{o} refers to oligomycin, which artificially induces state 4 by blocking the ATPase complex V). COX provides a measure of respiration through complex IV (or cytochrome oxidase, COX), using TMPD and ascorbate as electron donors. COXc is the respiration flux rate in the presence of TMPD, ascorbate and saturating cytochrome c. The ratio of COXc/COX provides a measure of membrane stability as cytochrome c can be lost from the inner mitochondrial membrane due to damage to the outer mitochondrial membrane additional cytochrome C results in increased flux.

Approximately 2.5-4 mg wet weight of fibres was used per assay. Mitochondria were assayed at a final concentration of 50 ug.mL⁻¹. Assays were repeated four times per sample for fibres.

Assays were calibrated by saturation prior to all assays for each assay and zeroed prior to assay sessions with sodium dithionite. A solubility coefficient of 0.92 was used for the assay media and fluctuations in ambient barometric pressure accounted for by the Oxygraph software. The stir bar speed was 750 rpm. Activities were calculated from the maximal flux rates following addition of the substrates and attainment of a steady state. Due to unequal variance non parametric statistics were used (Kruskal Wallis followed by Mann Whitney U).

There was a statistically significant depression of all measured flux rates when comparing untreated and treated controls. (See FIG. 20). Respiration flux through all complexes was depressed by approximately 40% in diabetic mitochondria relative to control mitochondria and fibres (results not shown). Treatment with triethylenetetramine disuccinate showed significant improvement in cytochrome C oxidase (as assayed against TMPD plus ascorbate) which increased relative to the flux rates found in the untreated diabetic. No significant effect of triethylenetetramine disuccinate treatment on the control treated mitochondria was detected.

### Example 8

### Effects of Polyamines on Left Ventricle Mitochondria of SHR Rats

Spontaneous Hypertensive Rats (SHR) and the matched rat control (Wistar-Kyoto (WKY)) rats were housed, kept in pairs and maintained on Teklad TB 2108 (Harlan UK) rat chow and tap water ab libitum. Systolic blood pressure in the rats was measured using an indirect tail cuff method to indicate hypertension. The SHR rats had a systolic blood pressure of 184±6.4 mmHg and the WKY rats had a systolic blood pressure of 165±11.1 mmHg. At seventeen months (starting weight for both the SHR and WKY rats was approximately 400 g each) the SHR and WKY rats were randomized into a further two groups (four groups in total); (1) SHR treated with triethylenetetramine disuccinate; (2) untreated SHR; (3) WKY treated with triethylenetetramine disuccinate; and (4) untreated WKY. The treated animals were administered triethylenetetramine disuccinate dissolved in Milli-Q water at a rate of 87.5 mg/rat/day for 12 weeks. The untreated rats received Milli-Q water ab libitum during the corresponding period. During the treatment period, no significant change was observed in the blood pressure of the rats.

The mitochondrial fibre extraction and the rates of respiration were carried out in accordance with the procedure set out in example 7 above.

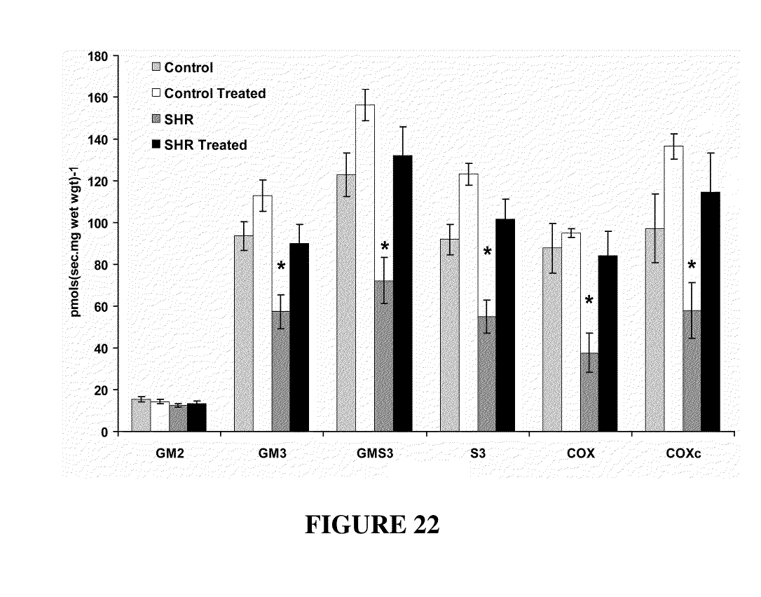

Except for GM2, there was an approximate 40% depression in respiratory flux through all complexes when comparing the untreated SHR model to the untreated WKY model. (See FIG. 22). Similarly, except for GM2, triethylenetetramine disuccinate treatment of the SHR and WKY models resulted in statistically significant improvements in flux through all complexes.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member or subgroup of members of the Markush group, and applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. A method for
the treatment of a mitochondria-associated disease by administration to a mammal in need thereof a composition comprising a therapeutically effective amount of a pharmaceutically acceptable copper (II) binding tetramine compound or a compound according to Formula (I) or Formula (II), and a pharmaceutically acceptable carrier.

2. The method of claim 1 wherein the tetramine is a linear or branched tetramine capable of binding copper (II) or a salt, active metabolite, derivative, or prodrug thereof.

3. The method of claim 2 wherein said linear or branched tetramine is a copper (II) chelator.

4. The method of claim 1 wherein the tetramine compound is a tetramine compound that is specific for Cu²⁺ over Cu⁺.

5. The method of claim 1 wherein the tetramine compound binds Cu²⁺ but does not bind Cu⁺.

6. The method of claim 2 wherein said linear or branched tetramine is selected from the group consisting of 2,3,2 tetramine, 2,2,2 tetramine, and 3,3,3 tetramine.

7. The method of claim 1 wherein said tetramine is triethylenetetramine.

8. The method of claim 1 wherein said tetramine is a triethylenetetramine salt.

9. The method of claim 8 wherein said triethylenetetramine salt is a succinate salt.

10. The method of claim 9 wherein said triethylenetetramine succinate salt is a triethylenetetramine disuccinate salt.

11. The method of claim 1 wherein said composition is a tablet or capsule for oral administration.

12. The method of claim 1 wherein said composition is a long-acting tablet or capsule for oral administration.

13. The method of claim 1 wherein said subject is a human.

14. The method of claim 13 wherein the subject does not have diabetes or cardiovascular disease.

15. The method of claim 1 wherein the mitochondria-associated disease is selected from the group consisting of a disease in which free radical mediated oxidative injury leads to mitochondrial degeneration; a disease in which cells inappropriately undergo apoptosis; stroke; an autoimmune disease; Alzheimer's disease; Parkinson's disease; dementia; chorea; and schizophrenia.

16. The method of claim 13 wherein the tetramine is a linear or branched tetramine capable of binding copper (II) or a salt, active metabolite, derivative, or prodrug thereof.

17. The method of claim 16 wherein said linear or branched tetramine is a copper (II) chelator.

18. The method of claim 17 wherein said linear or branched tetramine is selected from the group consisting of 2,3,2 tetramine, 2,2,2 tetramine, and 3,3,3 tetramine.

19. The method of claim 13 wherein said tetramine is triethylenetetramine.

20. The method of claim 13 wherein said tetramine is a triethylenetetramine salt.

21. The method of claim 20 wherein said triethylenetetramine salt is a succinate salt.
